SEQUENCE LISTING (8 sequences)

SEQ ID NO: 1 - DNA, 19 bp, Artificial Sequence
  Primer
ctgtgccttgggtggcttt

SEQ ID NO: 2 - DNA, 24 bp, Artificial Sequence
  Primer
aaggaaagaagtcagaaggcaaaa

SEQ ID NO: 3 - DNA, 34 bp, Artificial Sequence
  Probe
agctccaaattctttataagggtcgatgtccatg

SEQ ID NO: 4 - DNA, 19 bp, Artificial Sequence
  Primer
ctccccgtctgtgccttct

SEQ ID NO: 5 - DNA, 18 bp, Artificial Sequence
  Primer
gccccaaagccacccaag

SEQ ID NO: 6 - DNA, 27 bp, Artificial Sequence
  Probe
cgtcgcatggagaccaccgtgaacgcc

SEQ ID NO: 7 - DNA, 43 bp, Artificial Sequence
  HBV T7+Forward Primer
taatacgactcactatagggtttttcacctctgcctaatcatc

SEQ ID NO: 8 - DNA, 37 bp, Artificial Sequence
  HBV Reverse Primer
cctctagagcggccgcaaaaagttgcatggtgctggt

(19) United States
(12) Patent Application Publication
(10) Pub. No.: US 2023/0248723 A1
(43) Pub. Date: Aug. 10, 2023

(54) PYRROLO[2,3-B]PYRAZINE COMPOUNDS AS CCCDNA INHIBITORS FOR THE TREATMENT OF HEPATITIS B VIRUS (HBV) INFECTION

(71) Applicant: Hoffmann-La Roche Inc., Little Falls, NJ (US)

(72) Inventors: Jitao David ZHANG, Basel (CH); Xingchun HAN, Shanghai (CN); Song YANG, Shanghai (CN); Miriam TRIYATNI, Basel (CH); Brian LEONARD, Basel (CH); Angelina WALLIER, Basel (CH); Josephine SCHMALER, Basel (CH); Daniel TURLEY, Basel (CH); Philipp TROPBERGER, Basel (CH); Samuel CROSET, Basel (CH)

(21) Appl. No.: 16/769,284

(22) PCT Filed: Dec. 4, 2018
(86) PCT No.: PCT/EP2018/083491

§ 371 Date: Jun. 3, 2020

(30) Foreign Application Priority Data
Dec. 4, 2017 (WO) PCT/CN2017/114438

(51) Int. Cl. A61K 31/4985; A61P 31/20; G01N 33/50

(52) U.S. Cl. CPC A61K 31/4985; A61P 31/20; G01N 33/5067; G01N 33/5023

(57) ABSTRACT

The present invention relates to novel therapeutic agents against hepatitis B virus (HBV) infection, particularly inhibitors of viral covalently closed circular DNA (cccDNA) which is the key barrier for HBV cure. Accordingly, the invention provides the pyrrolo[2,3-b]pyrazine compounds of formula (I), as described and defined herein, for use in the treatment of HBV infection. The compounds provided herein are highly potent against HBV infection and enable an improved therapy, particularly of chronic HBV infection and HBV rebound. The present invention further relates to a novel screening assay for the identification of therapeutic agents against HBV infection, particularly cccDNA inhibitors, which is performed in hepatocyte-like cells that recapitulate the complete HBV life cycle following infection with patient-derived HBV.

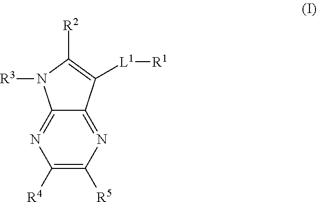

The present invention relates to novel therapeutic agents against hepatitis B virus (HBV) infection, particularly inhibitors of viral covalently closed circular DNA (cccDNA) that represents the key virological barrier for HBV cure. Accordingly, the invention provides the pyrrolo[2,3-b]pyrazine compounds of formula (I), as described and defined herein, for use in the treatment of HBV infection. The compounds provided herein are highly potent against HBV infection and enable an improved therapy, particularly of chronic HBV infection and HBV rebound. The present invention further relates to a novel screening assay for the identification of therapeutic agents against HBV infection, particularly cccDNA inhibitors, which is performed in hepatocyte-like cells that recapitulate the complete HBV life cycle following infection with patient-derived HBV.

Chronic hepatitis B virus (CHB) infection affects about 248 million individuals worldwide and causes about 686,000 deaths annually (GBD 2013; Schweitzer et al., 2015). Long term studies have shown that high viral load is associated with increased incidence of cirrhosis, hepatocellular carcinoma (HCC), and mortality (Chen et al., 2006; Iloeje et al., 2006; Iloeje et al., 2007; Liu et al., 2016). Thus, without curative treatment available, the majority of CHB infected individuals are at risk of developing cirrhosis and/or liver cancer.

Current standard of care (SOC) treatment effectively suppresses hepatitis B virus (HBV) DNA replication, but is not curative as it does not target the viral genetic template, i.e. covalently closed circular DNA (cccDNA), which is the virological barrier of HBV cure. Residing in the nucleus of infected hepatocytes, cccDNA gives rise to all HBV RNA transcripts needed for productive infection and is responsible for viral persistence during natural course of CHB infection (Locarnini & Zoulim, 2010). cccDNA is the source of viral rebound after cessation of treatment with SOC, virus reactivation following treatment with immunosuppressant, or after liver transplantation (Nassal, 2015; Kumar et al., 2016). Consequently, novel therapies that target cccDNA are highly needed.

However, drug discovery efforts to identify cccDNA inhibitors are challenging. HBV poorly propagates in vitro, and surrogate models e.g. hepatoma cell lines engineered to express cccDNA (Ladner et al., 1997; Guo et al., 2007) suffer from very low efficiency of cccDNA formation, such that the HBV transgene, and not cccDNA, acted as the dominant transcription template (Nassal, 2015, Zhang et al., 2016). The use of such systems to discover cccDNA inhibitors necessitated several counter screens in more relevant assays to confirm whether a compound acts on cccDNA, and not the transgene.

Furthermore, existing HBV systems do not capture HBV genotype (GT) diversity as they are mostly based on one GT. Worldwide, HBV exists as 10 GTs with about 40 subtypes; each GT has distinct properties in terms of geographical distribution, mode of transmission, and virological features. HBV GT is one of the important parameters in HBV pathogenesis as it affects viral pathogenesis, disease progression, response to treatment (e.g., with interferon-α), and risk factor for development of cirrhosis and HCC (Buster et al., 2009; Lin & Kao, 2017; Rajoriya et al., 2017).

As explained above, existing HBV systems mostly rely on a laboratory strain of a particular HBV GT, and thus do not capture HBV GT diversity in vivo. The use of a lab strain, instead of clinical HBV isolates, in drug discovery efforts may also lead to an overestimation of compound potency against the “real-world” HBV. Moreover, these systems are mostly based on hepatoma cell lines, e.g., the HepG2 cell line that has been shown to have poor resemblance to human hepatocytes (Uhlen et al., 2015).

It would thus be highly desirable to provide an HBV assay not only suitable for high-throughput screening (HTS), but also amenable for evaluating the breadth of compound potency against the “real-world” pathogens, i.e. clinical HBV isolates from various GTs. Collectively, there is an urgent need for improved HBV systems to identify novel cccDNA inhibitors. Such assays should ideally possess four salient features, namely i) robust, ii) recapitulate full virus life cycle following natural infection, iii) amenable for screening compounds against clinical HBV isolates, and iv) performed in physiological cell types such as primary cells or stem cells. The latter is considered as one of the crucial parameters to increase the translatability of preclinical findings into clinic (Eglen & Reisine, 2011; Vincent et al., 2015).

In practice, however, the limited supply, rapid dedifferentiation, and donor-to-donor variability of primary human hepatocytes (PHH) renders them unsuitable as a HTS platform (Frazcek et al., 2013; Mabit et al., 1996). In this regard, induced pluripotent stem cells (iPS) hold great promises as a surrogate for PHH due to their ability to differentiate into multiple disease-relevant cell types and their self-renewal potential (Eglen & Reisine, 2011; Shi et al., 2017; Ursu et al., 2017).

Screening assays for the identification of HBV cccDNA inhibitors have been described, e.g., in Cai D et al., Antiviral Res, 2016, 132:26-37 and Cai D et al., Antimicrob Agents Chemother, 2012, 56(8):4277-88. However, the corresponding screening method used a recombinant HepG2 cell line as a surrogate model for cccDNA; it is not known whether any compounds identified from such a system have been validated to inhibit cccDNA of clinical HBV isolates in a more relevant system, i.e. PHH. Certain pyrrolopyrazine compounds have been described in WO 2011/144585 as JAK and SYK inhibitors for the treatment of autoimmune and inflammatory diseases.

In the context of the present invention, a novel disease-relevant assay has been developed, i.e., stem cell-derived hepatocyte-like cells (referred to herein as HLC) or PHH infected with clinical HBV isolates from four (4) major GTs (A-D). This is the first HTS performed in primary-like cells that recapitulates complete HBV life cycle following infection with patient-derived HBV. In this assay, an iterative screening cascade, including early hit validation in PHH, led to the identification of compounds that target the elusive HBV cccDNA in the setting of natural infection. This approach demonstrated that iPS-derived hepatocyte-like cells (HLC) represent a paradigm change to discover novel cccDNA inhibitors, the key barrier for HBV cure.

This HLC platform for HBV drug discovery has been successfully used to discover novel and potent cccDNA inhibitors, following the HTS of a library of about 247,000 compounds. As also described in Example 1, a custom screening cascade was designed to address the inherent low levels of cccDNA in infected cells (0.1-1 copy/cell) on the basis that inhibitors of cccDNA can sequentially be identified through its more abundant, transcriptional products (HBsAg>>>HBeAg>>pgRNA>cccDNA). Thus, a multiplex assay (HBsAg, HBeAg, and albumin) was used as a primary HTS readout to identify dual HBsAg and HBeAg inhibitors and to exclude non-specific/toxic compounds (albumin is a counter tox screen). Hits were subsequently tested against pgRNA (proxy readout of cccDNA transcriptional activity), and finally, by a novel cccDNA-based digital PCR assay (dPCR assay). The advantage of this approach is that it allows direct assessment of compound activity on cccDNA using the low amount of cells (˜30,000 cells) present in a 384-well plate (a common plate format for HTS). Compounds that are active in the dPCR assay will then be confirmed in Southern Blot assay, a validated method for cccDNA detection but less sensitive and requires ˜15-fold amount of cells compared to dPCR.

This approach successfully identified novel HBV inhibitors, particularly novel cccDNA inhibitors that elicit at least partial cccDNA degradation, as shown for cccDNA of patient-derived HBV from major GTs (A-D) in PHH. Analysis of compound potency against various HBV markers demonstrated that a compound can be highly potent against HBsAg and HBeAg, however, its potency (HBsAg and HBeAg IC50) is not always correlated with its cccDNA IC50, highlighting the importance of direct measurement of cccDNA for accurate assessment of cccDNA inhibitor potency. In addition, the fact that compound potency may differ when tested against cell culture-derived HBV and/or in non-PHH cells, underscores the importance of compound testing in disease-relevant assays that better mimic conditions in vivo to increase their translatability in the clinic.

The present invention thus solves the problem of providing a disease-relevant screening assay that addresses the above-discussed needs, in particular a high-throughput screening assay that recapitulates the complete HBV life cycle using clinical isolates in HLC, and that allows to identify potent therapeutic agents against HBV infection, namely inhibitors of HBV cccDNA. The invention likewise solves the problem of providing novel and/or improved therapeutic agents for the treatment of HBV infection, particularly compounds that act as cccDNA inhibitors and are thus highly effective against HBV, including in the curative treatment of chronic HBV infection and in the treatment or suppression of HBV reactivation/rebound.

Accordingly, the present invention provides a compound of the following formula (I), or a pharmaceutically acceptable salt thereof, for use in treating a hepatitis B virus infection:

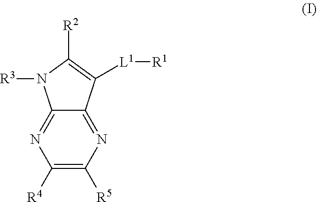

In formula (I), the group L¹ is selected from —CO—N(R^{L1})—, —N(R^{L1})—CO—, —CO—, —N(R^{L1})—, —C(═O)O—, —O—C(═O)—, —SO—, —SO₂—, —SO₂—N(R^{L1})—, and —N(R^{L1})—SO₂—.

Each R^{L1} is independently selected from hydrogen and C₁₋₅ alkyl.

R¹ is C₁₋₁₂ alkyl, C₂₋₁₂ alkenyl or C₂₋₁₂ alkynyl, wherein said alkyl, said alkenyl or said alkynyl is substituted with one or more groups R¹⁰, and further wherein said alkyl, said alkenyl or said alkynyl is optionally substituted with one or more groups R¹¹.

Each R¹⁰ is independently selected from —OH, —O(C₁₋₅ alkyl), and heterocyclyl having at least one oxygen ring atom.

Each R¹¹ is independently selected from —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CF₃, —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), carbocyclyl, and heterocyclyl, wherein said carbocyclyl and said heterocyclyl are each optionally substituted with one or more groups R¹²; and further wherein any two groups R¹¹ (if present) that are bound to the same carbon atom may optionally form, together with the carbon atom that they are attached to, a 5- to 8-membered carbocyclic or heterocyclic ring, wherein said 5- to 8-membered carbocyclic or heterocyclic ring is optionally substituted with one or more groups R¹².

Each R¹² is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CF₃, —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(CIs alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl).

R² is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CF₃, —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-carbocyclyl, and —(C₀₋₃ alkylene)-heterocyclyl, wherein the carbocyclyl moiety of said —(C₀₋₃ alkylene)-carbocyclyl and the heterocyclyl moiety of said-(C₀₋₃ alkylene)-heterocyclyl are each optionally substituted with one or more groups R¹².

R³ is selected from hydrogen, C₁₋₅ alkyl, and —CO(C₁₋₅ alkyl).

R⁴ and R⁵ are each independently selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CF₃, —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-carbocyclyl, and —(C₀₋₃ alkylene)-heterocyclyl, wherein the carbocyclyl moiety of said —(C₀₋₃ alkylene)-carbocyclyl and the heterocyclyl moiety of said-(C₀₋₃ alkylene)-heterocyclyl are each optionally substituted with one or more groups R¹².

The present invention further provides a pharmaceutical composition for use in treating a hepatitis B virus infection, wherein the pharmaceutical composition comprises a compound of formula (I) or a pharmaceutically acceptable salt thereof and optionally a pharmaceutically acceptable excipient.

The invention likewise relates to the use of a compound of formula (I) or a pharmaceutically acceptable salt thereof in the preparation of a medicament for the treatment of a hepatitis B virus infection.

Moreover, the present invention provides a method of treating a hepatitis B virus infection, the method comprising administering a compound of formula (I) or a pharmaceutically acceptable salt thereof (or a pharmaceutical composition that comprises a compound of formula (I) or a pharmaceutically acceptable salt thereof, and that optionally comprises a pharmaceutically acceptable excipient) to a subject (e.g., a human) in need thereof. The method particularly comprises the administration of a therapeutically effective amount of the compound of formula (I) or the pharmaceutically acceptable salt thereof to the subject.

The compounds according to the present invention are highly advantageous in the therapy of HBV infection, including also the suppression of HBV reactivation/rebound. In particular, the invention provides inhibitors of viral cccDNA, i.e. therapeutic agents that inhibit HBV cccDNA stability and/or its transcriptional activity, and thus provides the possibility of HBV cure. Moreover, the compounds of the present invention are considered to be particularly effective under actual clinical conditions, as also confirmed in the appended examples, in which the potent efficacy of exemplary compounds of formula (I) against four major HBV genotypes has been demonstrated.

The present invention thus particularly relates to a compound of formula (I) or a pharmaceutically acceptable salt thereof (or a pharmaceutical composition comprising a compound of formula (I) or a pharmaceutically acceptable salt thereof, and optionally a pharmaceutically acceptable excipient) for use as a cccDNA inhibitor in treating a hepatitis B virus (HBV) infection. The invention likewise refers to a compound of formula (I) or a pharmaceutically acceptable salt thereof (or a corresponding pharmaceutical composition, i.e. a pharmaceutical composition comprising a compound of formula (I) or a pharmaceutically acceptable salt thereof, and optionally a pharmaceutically acceptable excipient) for use in treating an HBV infection by inhibiting HBV cccDNA. Moreover, the present invention further relates to a compound of formula (I) or a pharmaceutically acceptable salt thereof (or a corresponding pharmaceutical composition) for use in treating an HBV infection by destabilizing HBV cccDNA. The hepatitis B virus infection to be treated in accordance with the present invention is not particularly limited. For example, it may be an infection with (or an infectious disease caused by) any one or more hepatitis B virus genotypes, such as, e.g., HBV/A (i.e., hepatitis B virus genotype A), HBV/B, HBV/C, HBV/D, HBV/E, HBV/F, HBV/G, HBV/H, HBV/I, and/or HBV/J, particularly any one or more of HBV/A, HBV/B, HBV/C, HBV/D, and/or HBV/E, more preferably any one or more of HBV/A, HBV/B, HBV/C, and/or HBV/D. While the HBV infection to be treated may further be, e.g., an acute HBV infection or a chronic HBV infection, the present invention particularly relates to the treatment of chronic HBV infection (including a chronic infection with, or a chronic infectious disease caused by, any one or more of the aforementioned HBV genotypes, such as, e.g., HBV/A, HBV/B, HBV/C, HBV/D, HBV/E, HBV/F, HBV/G, HBV/H, HBV/I, and/or HBV/J). The HBV infection to be treated in accordance with the invention may also be a fulminant (or severe) HBV infection. Moreover, the invention also relates to the treatment of an HBV infection (including, in particular, any of the aforementioned specific types of HBV infection) in an immunocompromised and/or HIV-positive subject or in an immunosuppressed subject (particularly a corresponding human subject).

After an infection with HBV, particularly after the treatment of an HBV infection with standard of care medication, the HBV genetic template, i.e. cccDNA, still persists in the subject's body, which can eventually lead to a reactivation or recurrence of the HBV infection. The phenomenon of HBV reactivation (or HBV recurrence, or HBV rebound) is well-known in the medical field and represents a serious risk for HBV patients (see, e.g., Roche B et al., Liver Int, 2011, 31 Suppl 1:104-10; Mastroianni C M et al., World J Gastroenterol, 2011, 17(34):3881-7; or Vierling J M, Clin Liver Dis, 2007, 11(4):727-59). The present invention provides compounds that can target viral cccDNA and, thus, can allow curing an HBV infection by inhibiting or destabilizing the HBV cccDNA. Accordingly, the present invention also relates to the treatment or suppression of HBV reactivation (or HBV recurrence or rebound) using a cccDNA inhibitor provided herein, particularly a compound of formula (I) or a pharmaceutically acceptable salt thereof.

The present invention thus also relates to a compound of formula (I) or a pharmaceutically acceptable salt thereof (or a corresponding pharmaceutical composition, i.e. a pharmaceutical composition comprising a compound of formula (I) or a pharmaceutically acceptable salt thereof, and optionally a pharmaceutically acceptable excipient) for use in treating or suppressing HBV reactivation. The invention likewise provides a compound of formula (I) or a pharmaceutically acceptable salt thereof (or a corresponding pharmaceutical composition) for use in treating or suppressing HBV recurrence (or the recurrence of an HBV infection). The present invention further refers to a compound of formula (I) or a pharmaceutically acceptable salt thereof (or a corresponding pharmaceutical composition) for use in treating or suppressing HBV rebound (or a rebound of an HBV infection). The treatment or suppression of HBV reactivation (or HBV recurrence, or HBV rebound) in accordance with the instant invention includes, in particular, the prophylactic treatment (i.e., prevention) of HBV reactivation (or HBV recurrence or rebound). The invention furthermore refers to the use of a compound of formula (I) or a pharmaceutically acceptable salt thereof (or a corresponding pharmaceutical composition) in the preparation of a medicament for the treatment or suppression of HBV reactivation, or for the treatment or suppression of HBV recurrence, or for the treatment or suppression of HBV rebound. Moreover, the invention likewise provides a method of treating or suppressing HBV reactivation (or HBV recurrence, or HBV rebound), the method comprising administering a compound of formula (I) or a pharmaceutically acceptable salt thereof (or a corresponding pharmaceutical composition) to a subject in need thereof.

The present invention furthermore relates to the use of a compound of formula (I) or a pharmaceutically acceptable salt thereof as an inhibitor of hepatitis B virus cccDNA (i.e., as an HBV cccDNA inhibitor) in research, particularly as a research tool compound for inhibiting HBV cccDNA. Accordingly, the invention refers to the in vitro use of a compound of formula (I) or a pharmaceutically acceptable salt thereof as an HBV cccDNA inhibitor and, in particular, to the in vitro use of a compound of formula (I) or a pharmaceutically acceptable salt thereof as a research tool compound acting as an HBV cccDNA inhibitor. The invention likewise relates to a method, particularly an in vitro method, of inhibiting HBV cccDNA (e.g., destabilizing or silencing HBV cccDNA), the method comprising the application of a compound of formula (I) or a pharmaceutically acceptable salt thereof. The invention further relates to a method of inhibiting HBV cccDNA, the method comprising applying a compound of formula (I) or a pharmaceutically acceptable salt thereof to a test sample (e.g., a biological sample) or a test animal (i.e., a non-human test animal). The invention also refers to a method, particularly an in vitro method, of inhibiting HBV cccDNA in a sample (e.g., a biological sample), the method comprising applying a compound of formula (I) or a pharmaceutically acceptable salt thereof to said sample. The present invention further provides a method of inhibiting HBV cccDNA, the method comprising contacting a test sample (e.g., a biological sample) or a test animal (i.e., a non-human test animal) with a compound of formula (I) or a pharmaceutically acceptable salt thereof. The terms “sample”, “test sample” and “biological sample” include, without being limited thereto: a cell, a cell culture or a cellular or subcellular extract; biopsied material obtained from an animal (e.g., a human), or an extract thereof; or blood, serum, plasma, saliva, urine, feces, or any other body fluid, or an extract thereof. It is to be understood that the term “in vitro” is used in this specific context in the sense of “outside a living human or animal body”, which includes, in particular, experiments performed with cells, cellular or subcellular extracts, and/or biological molecules in an artificial environment such as an aqueous solution or a culture medium which may be provided, e.g., in a flask, a test tube, a Petri dish, a microtiter plate, etc.

The instant invention also provides a method of identifying an inhibitor of HBV cccDNA, the method comprising:
providing stem cell-derived hepatocyte-like cells infected with HBV;
subjecting a test compound to the stem cell-derived hepatocyte-like cells infected with HBV;
determining the inhibitory effect of the test compound on HBsAg and HBeAg in the infected stem cell-derived hepatocyte-like cells;
optionally determining the inhibitory effect of the test compound on albumin in the infected stem cell-derived hepatocyte-like cells and, if the test compound has been found to inhibit albumin, excluding it from further testing;
if the test compound has been found to inhibit HBsAg and HBeAg, determining the inhibitory effect of the test compound on HBV pgRNA;
if the test compound has been found to inhibit HBV pgRNA, determining the inhibitory effect of the test compound on HBV cccDNA; and
if the test compound has been found to inhibit HBV cccDNA, selecting the test compound as an inhibitor of HBV cccDNA.

This screening method is highly advantageous in that it allows the identification of inhibitors of HBV cccDNA, particularly of compounds capable of destabilizing HBV cccDNA (i.e., HBV cccDNA destabilizers), using a physiological system that recapitulates the full HBV life cycle and uses clinical HBV isolates of multiple HBV genotypes, thereby capturing well the HBV genotype diversity found in the “real world”. The compounds identified with this method can be used for HBV therapy, as described herein in connection with the compounds of formula (I). In particular, the HBV cccDNA inhibitors thus identified can be used in the treatment (or cure) of an HBV infection (including, e.g., a chronic HBV infection), or in the treatment or suppression of HBV reactivation (or HBV rebound or recession). This screening method can also be referred to as an in vitro method of identifying an inhibitor of HBV cccDNA.

As described above, this method comprises a step of providing stem cell-derived hepatocyte-like cells infected with HBV (preferably with patient-derived HBV). The hepatocyte-like cells are derived from stem cells, particularly from induced stem cells, more preferably from induced pluripotent stem cells (iPS). The corresponding stem cells (such as the induced pluripotent stem cells) may be mammalian cells (e.g., mouse cells), and are preferably human cells (or have been generated from human cells). The term “stem cell-derived hepatocyte-like cells” thus refers to stem cells (particularly induced pluripotent stem cells) that have been differentiated/maturated into hepatocytes (or, in other words, into hepatocyte-like cells), and it is used herein synonymously with “stem cell-derived hepatocytes” or “hepatocytes obtained from stem cells” or “hepatocyte-like cells obtained from stem cells”.

The stem cell-derived hepatocyte-like cells infected with HBV are preferably infected with patient-derived HBV, particularly with HBV isolated from a clinical sample. While the HBV (or the patient-derived HBV) may be of any genotype (GT), it is preferred that two or more sets of stem cell-derived hepatocyte-like cells are used, wherein each set of cells is infected with a different HBV GT, and more preferably at least four sets of stem cell-derived hepatocyte-like cells are used, which are infected with patient-derived HBV GTs A, B, C and D, respectively.

The step of providing stem cell-derived hepatocyte-like cells infected with HBV preferably comprises (i.e., is preferably conducted by carrying out the following steps):
treating stem cells (preferably pluripotent stem cells, more preferably induced pluripotent stem cells) with a compound disclosed in WO 2014/140058 (preferably the compound MB-1 or MB-2, as depicted below, or a pharmaceutically acceptable salt thereof) to obtain stem cell-derived hepatocyte-like cells; and
infecting the cells thus obtained with HBV (preferably with patient-derived HBV, more preferably with patient-derived HBV of GT A, B, C or D) to obtain the stem cell-derived hepatocyte-like cells infected with HBV.

The above-mentioned “compound disclosed in WO 2014/140058” may be any compound of formula I as described and defined in WO 2014/140058, including any one of the specific/exemplary compounds described in this document or a pharmaceutically acceptable salt of any one of these compounds. Corresponding compounds are also described in US 2015/0197726 and US 2015/0158840. Preferably, the “compound disclosed in WO 2014/140058” is any one of the following compounds MB-1 to MB-7 or a pharmaceutically acceptable salt thereof:

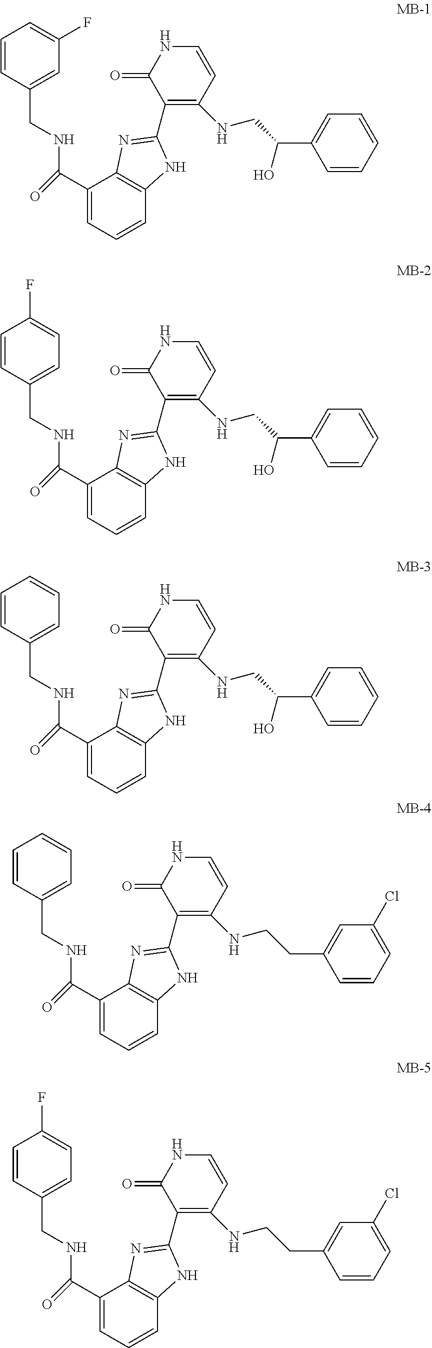

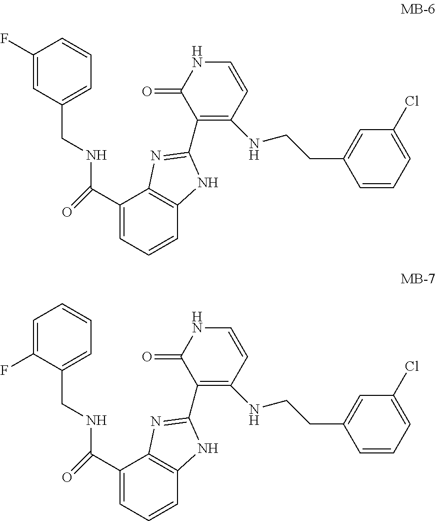 [SMILES: O=C(NCc1cccc(F)c1)c1cccc2[nH]c(-c3c(NCCc4cccc(Cl)c4)cc[nH]c3=O)nc12 ; O=C(NCc1ccccc1F)c1cccc2[nH]c(-c3c(NCCc4cccc(Cl)c4)cc[nH]c3=O)nc12]

The compound may also be a stereoisomer, particularly an enantiomer or a diastereomer, of any one of the above-depicted compounds MB-1, MB-2 or MB-3, or a pharmaceutically acceptable salt thereof. More preferably, the compound is MB-1 or MB-2, or a pharmaceutically acceptable salt thereof, and even more preferably it is MB-1 or a pharmaceutically acceptable salt thereof.

Accordingly, it is particularly preferred that the stem cells (or the induced pluripotent stem cells) are treated with the compound MB-1 or MB-2 or a pharmaceutically acceptable salt thereof (even more preferably with the compound MB-1 or a pharmaceutically acceptable salt thereof) to obtain stem cell-derived hepatocyte-like cells, and that the cells thus obtained are infected with HBV (preferably with a clinical HBV isolate of HBV genotype A, B, C or D) to obtain the stem cell-derived hepatocyte-like cells infected with HBV.

It is preferred that separate sets of the stem cell-derived hepatocyte-like cells are each individually infected with HBV of a different genotype. Preferably, sets of cells are infected with two or more HBV genotypes, more preferably with three or more HBV genotypes, more preferably with four or more HBV genotypes, even more preferably with five or more HBV genotypes, and yet even more preferably with six or more HBV genotypes. The HBV genotypes may be selected from the HBV genotypes A, B, C, D, E, F, G, H, I and J, preferably from the HBV genotypes A, B, C, D, E and F. It is particularly preferred that separate sets of the stem cell-derived hepatocyte-like cells are individually infected with clinical HBV isolates from at least the HBV genotypes A, B, C and D, and even more preferably with clinical HBV isolates from at least the HBV genotypes A, B, C, D, E and F. The test compound can be subjected to each of the various sets of stem cell-derived hepatocyte-like cells (each set of cells being infected with HBV of a different specific GT) in order to test several different HBV GTs. Thus, while the cells would be amenable for infection with all HBV genotypes, it is preferred that for compound testing, one set of cells is infected with only one genotype, so that, for instance, 10 different infection experiments would be needed to test a compound against all 10 HBV genotypes.

The method further comprises a step of subjecting a test compound to the stem cell-derived hepatocyte-like cells infected with HBV (preferably to at least four sets of stem cell-derived hepatocyte-like cells, said sets of cells being infected with patient-derived HBV of genotype A, B, C, and D, respectively). Typically, a plurality of test compounds (e.g., at least about 100 test compounds, or at least about 1000 test compounds, or at least about 10,000 test compounds, or at least about 100,000 test compounds) will be subjected simultaneously to the infected stem cell-derived hepatocyte-like cells. In principle, any compound can be employed as a test compound, including in particular small molecular compounds (e.g., compounds having a molecular weight of equal to or less than about 900 Da, preferably a molecular weight of equal to or less than about 500 Da).

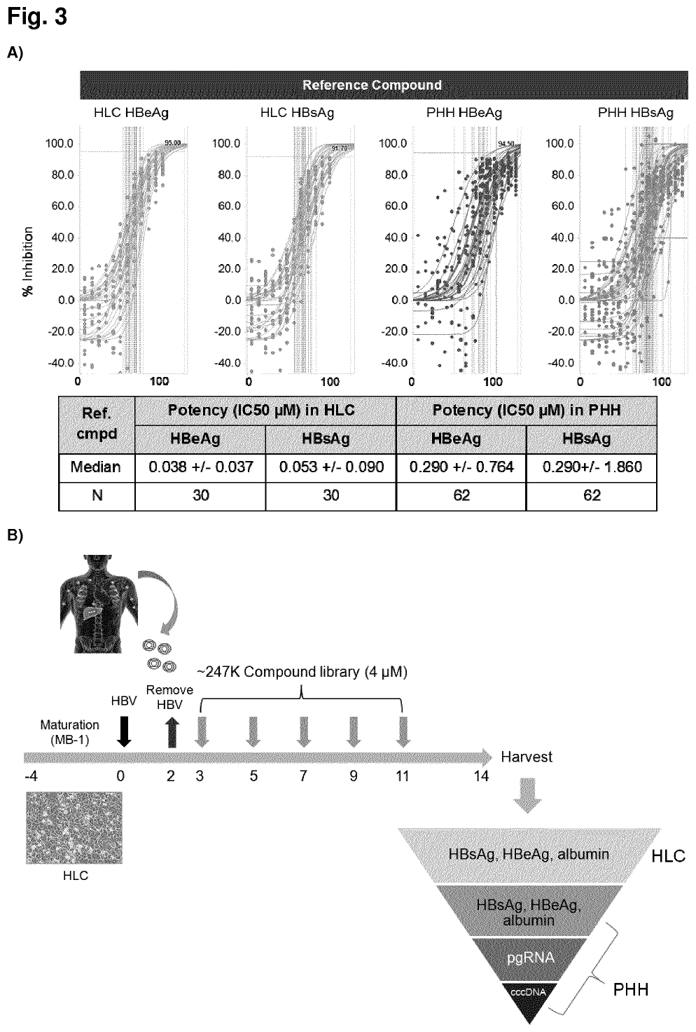

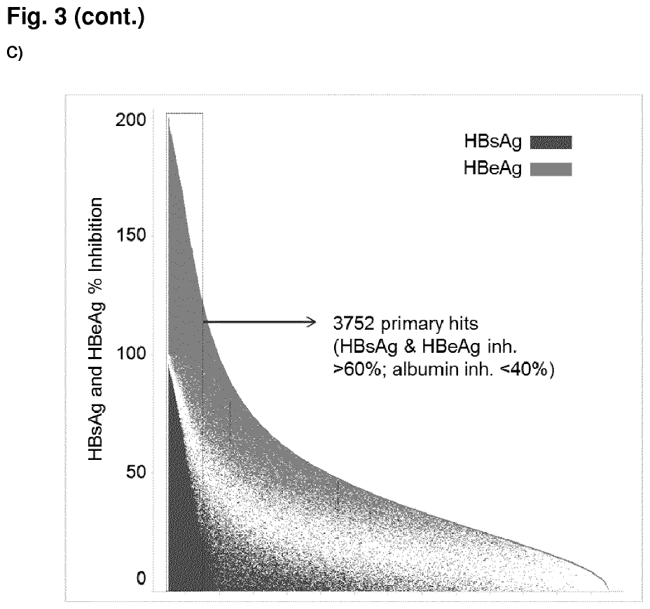

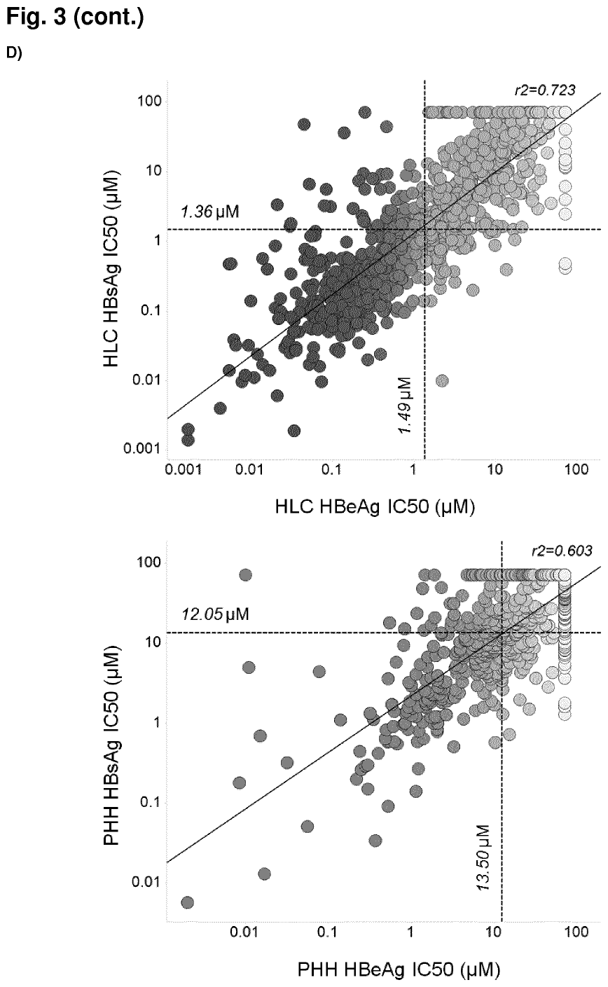

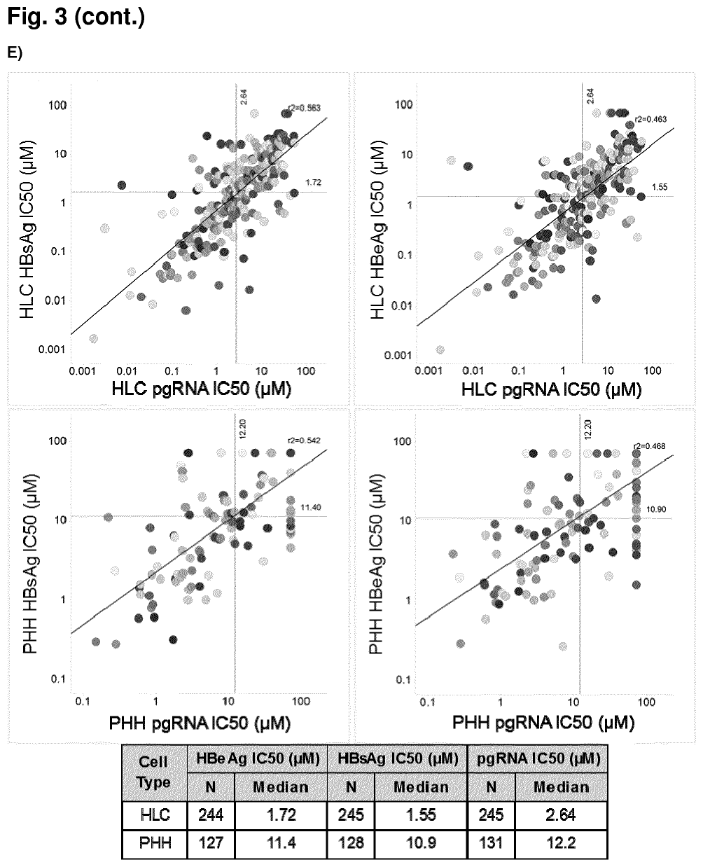

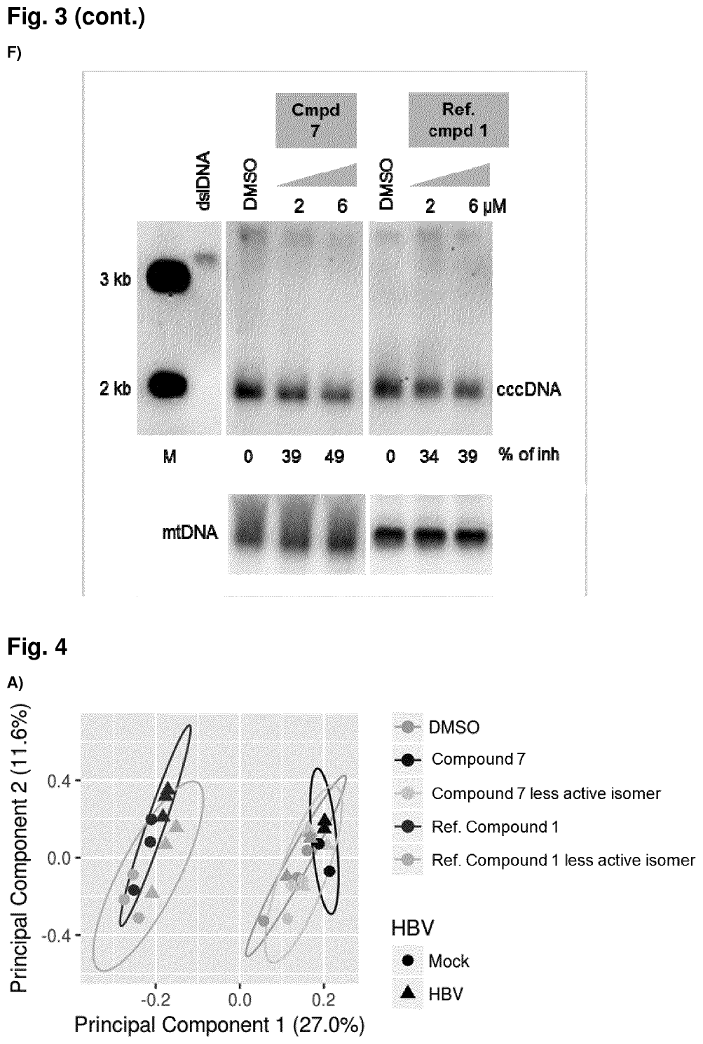

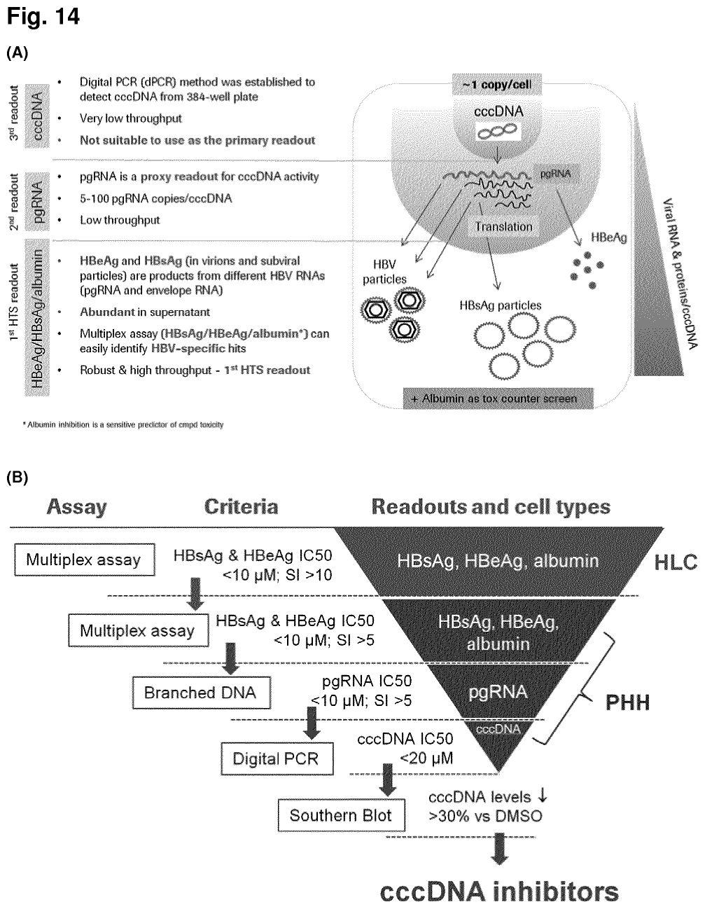

Following the step of subjecting a test compound (or a plurality of test compounds) to the stem cell-derived hepatocyte-like cells infected with HBV, the method comprises a cascade of steps in which the inhibitory effect (or inhibitory activity) of the respective test compound(s) against (i) HBsAg and HBeAg, (ii) pgRNA, and (iii) cccDNA is determined using the HBV-infected stem cell-derived hepatocyte-like cells (as also illustrated in FIGS. 3B, 14A and 14B). The sequential determination of a test compound's inhibitory effect on these HBV infection markers/targets allows to first select only such test compounds that inhibit both HBsAg and HBeAg (while excluding any test compound that does not inhibit HBsAg and HBeAg from further testing), then to select only such test compounds that additionally inhibit also pgRNA (while excluding any test compound that does not inhibit pgRNA from further testing), and then to select only such test compounds that also inhibit cccDNA. The inhibitory effect of a test compound on HBsAg and HBeAg, on pgRNA, and/or on cccDNA can be determined, e.g., as described in Example 1. In particular, the effect of a test compound on the expression and/or secretion of the viral proteins HBsAg and HBeAg can be assessed in order to determine the compound's inhibitory effect on HBsAg and HBeAg. Moreover, the effect of a test compound on HBV pregenome RNA (pgRNA) levels, e.g., in cell supernatants or in cell lysates can be assessed in order to determine the compound's inhibitory effect on pgRNA. The inhibitory effect of a test compound on cccDNA can likewise be determined, e.g., by assessing the effect of the test compound on cccDNA levels (cccDNA copy numbers) in cell lysates; this can be done, e.g., by using a PCR-based assay, particularly by digital PCR (e.g., as described in Example 1).

The method may optionally comprise a step of determining the inhibitory effect of the test compound on albumin in the infected stem cell-derived hepatocyte-like cells and, if the test compound has been found to inhibit albumin, excluding it from further testing. This step can be conducted simultaneously with the above-discussed step of determining the inhibitory effect of the test compound on HBsAg and HBeAg (e.g., using a multiplex assay as described in Example 1), and it is advantageous in that it allows to exclude non-specific and/or toxic compounds from further testing and, thus, to identify/obtain cccDNA inhibitors that are safe and well tolerable.

The method may further comprise a step of subjecting the test compound to PHH (also infected with HBV, as described herein with respect to the stem cell-derived hepatocyte-like cells) and determining its inhibitory effect against (i) HBsAg and HBeAg, and/or (ii) pgRNA, and/or (iii) cccDNA in order to confirm the test compound's activity. Depending on cell availability, PHH testing can be initiated after a test compound has been found to show dual activity against HBsAg and HBeAg in stem cell-derived hepatocyte-like cells, or after it has been found to show activity against pgRNA in stem cell-derived hepatocyte-like cells, or after it has been found to show activity against cccDNA in stem cell-derived hepatocyte-like cells. Thus, for example, if a test compound been found to show dual activity against HBsAg and HBeAg in stem cell-derived hepatocyte-like cells but not in PHH, it can be excluded from further testing.

A test compound that has been found to inhibit HBV cccDNA in this method can be selected/identified as an inhibitor of HBV cccDNA, particularly as an HBV cccDNA destabilizer. The above-described method can also be used to identify a broader range of highly potent therapeutic agents against HBV infection, including compounds that reduce, inhibit or silence the transcription of cccDNA but do not necessarily destabilize (or elicit the degradation of) HBV cccDNA. Thus, if a test compound has been found to inhibit HBV pgRNA (using this method), it can be selected as a therapeutic agent against HBV infection. In summary, this method allows identifying not only cccDNA destabilizers but also potential cccDNA silencers. The corresponding method can also be referred to as a method of identifying a therapeutic agent against HBV infection.

The compound of formula (I) will be described in more detail in the following:

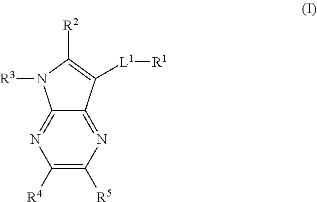

In formula (I), the group L¹ is selected from —CO—N(R^{L1})—, —N(R^{L1})—CO—, —CO—, —N(R^{L1})—, —C(═O)O—, —O—C(═O)—, —SO—, —SO₂—, —SO₂—N(R^{L1})—, and —N(R^{L1})—SO₂—.

Preferably, L¹ is selected from —CO—N(R^{L1})—, —N(R^{L1})—CO—, —C(═O)O—, —O—C(═O)—, —SO₂—N(R^{L1})—, and —N(R^{L1})—SO—. More preferably, L¹ is —CO—N(R^{L1})— or —N(R^{L1})—CO—, wherein the group —CO—N(R^{L1})— is bound via its carbon atom to the ring carbon atom of the pyrrolo[2,3-b]pyrazine moiety shown in formula (I) and is bound via its nitrogen atom to the group R¹, and wherein the group —N(R^{L1})—CO— is bound via its nitrogen atom to the ring carbon atom of the pyrrolo[2,3-b]pyrazine moiety shown in formula (I) and is bound via its carbon atom to the group R¹. Even more preferably, L¹ is —CO—N(R^{L1})—.

Each R^{L1} is independently selected from hydrogen and C₁₋₅ alkyl. Preferably, each R^{L1} is independently selected from hydrogen, methyl, and ethyl. More preferably, each R^{L1} is independently selected from hydrogen and methyl. Even more preferably, each R^{L1} is hydrogen.

Accordingly, it is particularly preferred that L¹ is —CO—N(R^{L1})—, wherein R^{L1} is hydrogen or C₁₋₅ alkyl (more preferably wherein R^{L1} is hydrogen or methyl, and even more preferably wherein R^{L1} is hydrogen), and the compound of formula (I) has the following structure:

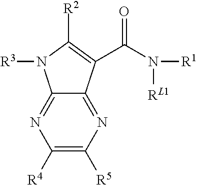

R¹ is C₁₋₁₂ alkyl, C₂₋₁₂ alkenyl or C₂₋₁₂ alkynyl, wherein said alkyl, said alkenyl or said alkynyl is substituted with one or more (e.g., one, two or three) groups R¹⁰, and further wherein said alkyl, said alkenyl or said alkynyl is optionally substituted with one or more (e.g., one, two or three) groups R¹¹.

Preferably, R¹ is C₁₋₁₂ alkyl, wherein said alkyl is substituted with one or more (e.g., one, two or three) groups R¹⁰, and further wherein said alkyl is optionally substituted with one or more (e.g., one, two or three) groups R¹¹. More preferably, R¹ is C₂₋₁₀ alkyl, wherein said alkyl is substituted with one or more groups R¹⁰, and further wherein said alkyl is optionally substituted with one or more groups R¹¹. Even more preferably, R¹ is —C(R¹³)(R¹³)—C(R¹³)(R¹³)—R¹⁰, wherein each R¹³ is independently selected from hydrogen and C₁₋₄ alkyl, provided that the total number of carbon atoms in all groups R¹³ together is equal to or less than 8, wherein each R¹³ is optionally substituted with one or more groups R¹⁰, and wherein each R¹³ is optionally further substituted with one or more groups R¹¹. Yet even more preferably, R¹ is —C(R¹³)(R¹³)—C(R¹³)(R¹³)—R¹⁰, wherein each R¹³ is independently selected from hydrogen, methyl and ethyl, wherein each R¹³ is optionally substituted with one or more (e.g., one or two) groups R¹⁰, and wherein each R¹³ is optionally further substituted with one or more (e.g., one, two or three) groups R¹¹. Particularly preferred examples of R¹ include, without being limited thereto, —CH(—CH₂OH)—CH₂—OH, —CH(—CH₃)—CH₂—OH, —C(—CH₃)(—CH₃)—C(—CH₃)(—CH₃)—OH, -(1-(hydroxymethyl)cyclopentan-1-yl), —CH(—CH₂CH₃)—CH₂—OH, —CH(—CH₂CH₂OH)—C(—CH₃)(—CH₃)—OH, or —C(—CH₃)(—CH₃)—CH₂—OH.

Each R¹⁰ is independently selected from —OH, —O(C₁₋₅ alkyl), and heterocyclyl having at least one oxygen ring atom. Preferably, each R¹⁰ is independently selected from —OH, —O(C₁₋₅ alkyl), and heterocycloalkyl having at least one oxygen ring atom. More preferably, each R¹⁰ is independently selected from —OH and —O(C₁₋₅ alkyl), even more preferably each R¹⁰ is independently selected from —OH, —OCH₃, and —OCH₂CH₃, and yet even more preferably each R¹⁰ is independently selected from —OH and —OCH₃. Most preferably, each R¹⁰ is —OH.

As specified above, R¹⁰ may be heterocyclyl having at least one oxygen ring atom. If R¹⁰ is heterocyclyl having at least one oxygen ring atom, then it is preferred that said heterocyclyl is a heterocycloalkyl having at least one oxygen ring atom. It is furthermore preferred that said heterocyclyl or said heterocycloalkyl has from 5 to 10 ring atoms (including at least one oxygen ring atom); more preferably, it is monocyclic and has 5, 6 or 7 ring atoms, particularly 5 or 6 ring atoms. The ring atoms of the heterocyclyl or the heterocycloalkyl (including the aforementioned 5 to 10 ring atoms, or the 5, 6 or 7 ring atoms, or the 5 or 6 ring atoms) preferably include 1 oxygen ring atom and x further heteroatoms selected independently from oxygen, nitrogen and sulfur, wherein x is 0, 1 or 2, and wherein the remaining ring atoms are carbon atoms. Examples of such heterocyclyl or heterocycloalkyl groups include, in particular, tetrahydrofuranyl (e.g., tetrahydrofuran-2-yl or tetrahydrofuran-3-yl), tetrahydropyranyl (e.g., tetrahydropyran-2-yl, tetrahydropyran-3-yl, or tetrahydropyran-4-yl), or morpholinyl (e.g., morpholin-4-yl).

Each R¹¹ is independently selected from —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CF₃, —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), carbocyclyl, and heterocyclyl, wherein said carbocyclyl and said heterocyclyl are each optionally substituted with one or more (e.g., one, two or three) groups R¹²; and further wherein any two groups R¹¹ (if present) that are bound to the same carbon atom may optionally form, together with the carbon atom that they are attached to, a 5- to 8-membered carbocyclic or heterocyclic ring, wherein said 5- to 8-membered carbocyclic or heterocyclic ring is optionally substituted with one or more (e.g., one, two or three) groups R¹².

Preferably, each R¹¹ is independently selected from —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CF₃, and —CN; and further wherein any two groups R¹¹ (if present) that are bound to the same carbon atom may optionally form, together with the carbon atom that they are attached to, a 5- or 6-membered carbocyclic or heterocyclic ring, wherein said 5- or 6-membered carbocyclic or heterocyclic ring is optionally substituted with one or more groups R¹².

If two groups R¹¹ are bound to the same carbon atom and if they form, together with the carbon atom that they are attached to, a 5- to 8-membered carbocyclic or heterocyclic ring (or, in particular, a 5- or 6-membered carbocyclic or heterocyclic ring), wherein said ring is optionally substituted with one or more groups R¹², then it is preferred that said ring is saturated. More preferably, said ring is a saturated 5- or 6-membered carbocyclic or heterocyclic ring which is optionally substituted with one or more groups R¹². The aforementioned saturated 5- or 6-membered heterocyclic ring preferably contains 1 or 2 oxygen ring atoms, with all remaining ring atoms being carbon atoms. Examples of a corresponding carbocyclic or heterocyclic ring include, in particular, a Cyclopentyl ring, a cyclohexyl ring, a tetrahydrofuranyl ring, or a tetrahydropyranyl ring (wherein each of the aforementioned rings may be optionally substituted with one or more groups R¹²).

Each R¹² is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CF₃, —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl).

Preferably, each R¹² is independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CF₃, —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), and —N(C₁₋₅ alkyl)-SO₂—(C₁₋₅ alkyl). More preferably, each R¹² is independently selected from C₁₋₅ alkyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CF₃, and —CN.

R² is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CF₃, —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-carbocyclyl, and —(C₀₋₃ alkylene)-heterocyclyl, wherein the carbocyclyl moiety of said —(C₀₋₃ alkylene)-carbocyclyl and the heterocyclyl moiety of said-(Cos alkylene)-heterocyclyl are each optionally substituted with one or more (e.g., one, two or three) groups R¹².

Preferably, R² is selected from hydrogen, C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CF₃, —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), and —N(C₁₋₅ alkyl)-SO₂—(C₁₋₅ alkyl). More preferably, R² is selected from hydrogen, C₁₋₅ alkyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CF₃, and —CN. Even more preferably, R² is hydrogen.

R³ is selected from hydrogen, C₁₋₅ alkyl, and —CO(C₁₋₅ alkyl).

Preferably, R³ is hydrogen or C₁₋₅ alkyl. More preferably, R³ is hydrogen, methyl or ethyl. Even more preferably, R³ is hydrogen.

R⁴ and R⁵ are each independently selected from hydrogen, C₁₋₅ alkyl, C₂₋₅ alkenyl, C₂₋₅ alkynyl, —(Cos alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(Cos alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CF₃, —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-carbocyclyl, and —(C₀₋₃ alkylene)-heterocyclyl, wherein the carbocyclyl moiety of said —(C₀₋₃ alkylene)-carbocyclyl and the heterocyclyl moiety of said-(C₀₋₃ alkylene)-heterocyclyl are each optionally substituted with one or more (e.g., one, two or three) groups R¹².

Preferably, one of R⁴ and R⁵ is carbocyclyl or heterocyclyl, wherein said carbocyclyl or said heterocyclyl is optionally substituted with one or more groups R¹², and the other one of R⁴ and R⁵ is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CF₃, —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-carbocyclyl, and —(C₀₋₃ alkylene)-heterocyclyl, wherein the carbocyclyl moiety of said —(C₀₋₃ alkylene)-carbocyclyl and the heterocyclyl moiety of said-(C₀₋₃ alkylene)-heterocyclyl are each optionally substituted with one or more groups R¹². More preferably, R⁵ is cycloalkyl, and R⁴ is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CF₃, —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-carbocyclyl, and —(C₀₋₃ alkylene)-heterocyclyl, wherein the carbocyclyl moiety of said —(C₀₋₃ alkylene)-carbocyclyl and the heterocyclyl moiety of said-(C₀₋₃ alkylene)-heterocyclyl are each optionally substituted with one or more groups R¹². Even more preferably, R⁵ is C₃₋₇ cycloalkyl (e.g., cyclopropyl, cyclobutyl, cyclopentyl, cyclohexyl, or cycloheptyl; particularly cyclopropyl), and R⁴ is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CF₃, —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-carbocyclyl, and —(C₀₋₃ alkylene)-heterocyclyl, wherein the carbocyclyl moiety of said —(C₀₋₃ alkylene)-carbocyclyl and the heterocyclyl moiety of said-(C₀₋₃ alkylene)-heterocyclyl are each optionally substituted with one or more groups R¹²; wherein R⁴ is more preferably selected from hydrogen, C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CF₃, —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), carbocyclyl, and heterocyclyl, wherein said carbocyclyl and said heterocyclyl are each optionally substituted with one or more groups R¹²; wherein R⁴ is even more preferably selected from hydrogen, C₁₋₅ alkyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CF₃, and —CN; and wherein R⁴ is still more preferably hydrogen. Even more preferably, R⁵ is cyclopropyl, and R⁴ is selected from hydrogen, C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CF₃, —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), carbocyclyl, and heterocyclyl, wherein said carbocyclyl and said heterocyclyl are each optionally substituted with one or more groups R¹²; wherein R⁴ is more preferably selected from hydrogen, C₁₋₅ alkyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CF₃, and —CN; and wherein R⁴ is still more preferably hydrogen. Still more preferably, R⁵ is cyclopropyl, and R⁴ is hydrogen.

It is particularly preferred that the compound of formula (I) is a compound of the following formula (II) or a pharmaceutically acceptable salt thereof:

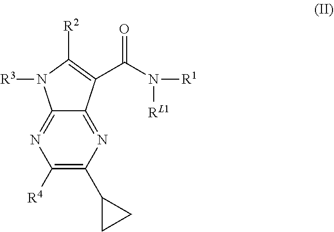

wherein the groups/variables comprised in formula (II), including in particular R¹, R^{L1}, R², R³ and R⁴, have the same meanings, including the same preferred meanings, as described and defined herein for the corresponding groups/variables comprised in formula (I).

The compound of formula (I) or (II) may be, e.g., any one of the specific compounds described in the examples section of this specification, either in non-salt form (e.g., free base/acid form) or as a pharmaceutically acceptable salt of the respective compound.

In particular, examples of the compounds of formula (I) or (II) include the following compounds as well as pharmaceutically acceptable salts of any one of these compounds:

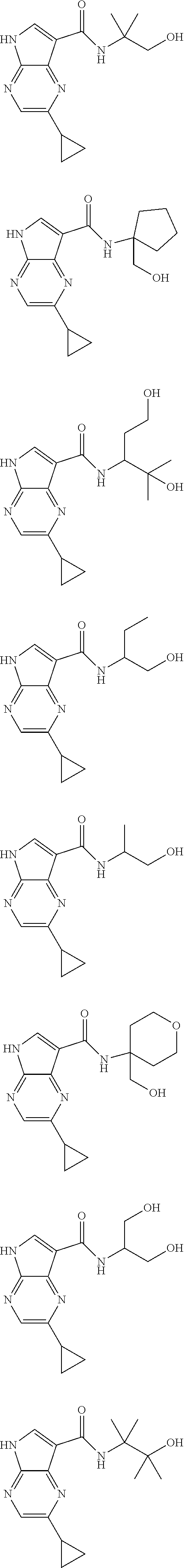

Preferred examples of the compounds of formula (I) or (II) include, in particular, the following compounds as well as pharmaceutically acceptable salts thereof:

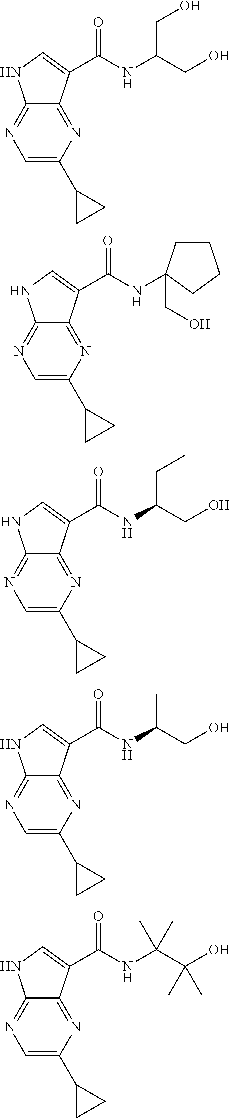

A particularly preferred exemplary compound of formula (I) or (II) is a compound of the following formula (which is also referred to herein as “compound 7”), or a pharmaceutically acceptable salt thereof:

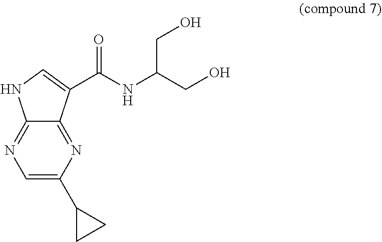 [SMILES: O=C(NC(CO)CO)c1c[nH]c2ncc(C3CC3)nc12]

The compounds of formula (I) can be prepared by methods known in the field of synthetic chemistry. For example, these compounds can be prepared in accordance with or in analogy to any of the synthetic routes described in WO 2011/144585 (the content of which is herewith incorporated by reference in its entirety), particularly on pages 93 to 101 and/or in the working examples of WO 2011/144585.

The following definitions apply throughout the present specification and the claims, unless specifically indicated otherwise.

The term “hydrocarbon group” refers to a group consisting of carbon atoms and hydrogen atoms.

The term “alicyclic” is used in connection with cyclic groups and denotes that the corresponding cyclic group is non-aromatic.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₅ alkyl” denotes an alkyl group having 1 to 5 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₄ alkyl, more preferably to methyl or ethyl, and even more preferably to methyl.

As used herein, the term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₅ alkenyl” denotes an alkenyl group having 2 to 5 carbon atoms. Preferred exemplary alkenyl groups are ethenyl, propenyl (e.g., prop-1-en-1-yl, prop-1-en-2-yl, or prop-2-en-1-yl), butenyl, butadienyl (e.g., buta-1,3-dien-1-yl or buta-1,3-dien-2-yl), pentenyl, or pentadienyl (e.g., isoprenyl). Unless defined otherwise, the term “alkenyl” preferably refers to C₂₋₄ alkenyl.

As used herein, the term “alkynyl” refers to a monovalent unsaturated acyclic hydrocarbon group which may be linear or branched and comprises one or more (e.g., one or two) carbon-to-carbon triple bonds and optionally one or more (e.g., one or two) carbon-to-carbon double bonds. The term “C₂₋₅ alkynyl” denotes an alkynyl group having 2 to 5 carbon atoms. Preferred exemplary alkynyl groups are ethynyl, propynyl (e.g., propargyl), or butynyl. Unless defined otherwise, the term “alkynyl” preferably refers to C₂₋₄ alkynyl.

As used herein, the term “alkylene” refers to an alkanediyl group, i.e. a divalent saturated acyclic hydrocarbon group which may be linear or branched. A “C₁₋₅ alkylene” denotes an alkylene group having 1 to 5 carbon atoms, and the term “C₀₋₃ alkylene” indicates that a covalent bond (corresponding to the option “C₀ alkylene”) or a C₁₋₃ alkylene is present. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—). Unless defined otherwise, the term “alkylene” preferably refers to C₁₋₄ alkylene (including, in particular, linear C₁₋₄ alkylene), more preferably to methylene or ethylene, and even more preferably to methylene.

As used herein, the term “carbocyclyl” (or “carbocyclic ring”) refers to a hydrocarbon ring group, including monocyclic rings as well as bridged ring, spiro ring and/or fused ring systems (which may be composed, e.g., of two or three rings), wherein said ring group may be saturated, partially unsaturated (i.e., unsaturated but not aromatic) or aromatic. Unless defined otherwise, “carbocyclyl” (or “carbocyclic ring”) preferably refers to aryl, cycloalkyl or cycloalkenyl.

As used herein, the term “heterocyclyl” (or “heterocyclic ring”) refers to a ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. For example, each heteroatom-containing ring comprised in said ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. Unless defined otherwise, “heterocyclyl” (or “heterocyclic ring”) preferably refers to heteroaryl, heterocycloalkyl or heterocycloalkenyl.

As used herein, the term “aryl” refers to an aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). “Aryl” may, e.g., refer to phenyl, naphthyl, dialinyl (i.e., 1,2-dihydronaphthyl), tetralinyl (i.e., 1,2,3,4-tetrahydronaphthyl), indanyl, indenyl (e.g., 1H-indenyl), anthracenyl, phenanthrenyl, 9H-fluorenyl, or azulenyl. Unless defined otherwise, an “aryl” preferably has 6 to 14 ring atoms, more preferably 6 to 10 ring atoms, even more preferably refers to phenyl or naphthyl, and most preferably refers to phenyl.

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said aromatic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromanyl, chromenyl (e.g., 2H-1-benzopyranyl or 4H-1-benzopyranyl), isochromenyl (e.g., 1H-2-benzopyranyl), chromonyl, xanthenyl, phenoxathiinyl, pyrrolyl (e.g., 1H-pyrrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolyl (e.g., 3H-indolyl), isoindolyl, indazolyl, indolizinyl, purinyl, quinolyl, isoquinolyl, phthalazinyl, naphthyridinyl, quinoxalinyl, cinnolinyl, pteridinyl, carbazolyl, O-carbolinyl, phenanthridinyl, acridinyl, perimidinyl, phenanthrolinyl (e.g., [1,10]phenanthrolinyl, [1,7]phenanthrolinyl, or [4,7]phenanthrolinyl), phenazinyl, thiazolyl, isothiazolyl, phenothiazinyl, oxazolyl, isoxazolyl, oxadiazolyl (e.g., 1,2,4-oxadiazolyl, 1,2,5-oxadiazolyl (i.e., furazanyl), or 1,3,4-oxadiazolyl), thiadiazolyl (e.g., 1,2,4-thiadiazolyl, 1,2,5-thiadiazolyl, or 1,3,4-thiadiazolyl), phenoxazinyl, pyrazolo[1,5-a]pyrimidinyl (e.g., pyrazolo[1,5-a]pyrimidin-3-yl), 1,2-benzoisoxazol-3-yl, benzothiazolyl, benzothiadiazolyl, benzoxazolyl, benzisoxazolyl, benzimidazolyl, benzo[b]thiophenyl (i.e., benzothienyl), triazolyl (e.g., 1H-1,2,3-triazolyl, 2H-1,2,3-triazolyl, 1H-1,2,4-triazolyl, or 4H-1,2,4-triazolyl), benzotriazolyl, 1H-tetrazolyl, 2H-tetrazolyl, triazinyl (e.g., 1,2,3-triazinyl, 1,2,4-triazinyl, or 1,3,5-triazinyl), furo[2,3-c]pyridinyl, dihydrofuropyridinyl (e.g., 2,3-dihydrofuro[2,3-c]pyridinyl or 1,3-dihydrofuro[3,4-c]pyridinyl), imidazopyridinyl (e.g., imidazo[1,2-a]pyridinyl or imidazo[3,2-a]pyridinyl), quinazolinyl, thienopyridinyl, tetrahydrothienopyridinyl (e.g., 4,5,6,7-tetrahydrothieno[3,2-c]pyridinyl), dibenzofuranyl, 1,3-benzodioxolyl, benzodioxanyl (e.g., 1,3-benzodioxanyl or 1,4-benzodioxanyl), or coumarinyl. Unless defined otherwise, the term “heteroaryl” preferably refers to a 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heteroaryl” include pyridinyl (e.g., 2-pyridyl, 3-pyridyl, or 4-pyridyl), imidazolyl, thiazolyl, 1H-tetrazolyl, 2H-tetrazolyl, thienyl (i.e., thiophenyl), or pyrimidinyl. As used herein, the term “cycloalkyl” refers to a saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, decalinyl (i.e., decahydronaphthyl), or adamantyl. Unless defined otherwise, “cycloalkyl” preferably refers to a C₃₋₁₁ cycloalkyl, and more preferably refers to a C₃₋₇ cycloalkyl. A particularly preferred “cycloalkyl” is a monocyclic saturated hydrocarbon ring having 3 to 7 ring members. Moreover, unless defined otherwise, particularly preferred examples of a “cycloalkyl” include cyclohexyl or cyclopropyl, particularly cyclohexyl.

As used herein, the term “heterocycloalkyl” refers to a saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkyl” may, e.g., refer to aziridinyl, azetidinyl, pyrrolidinyl, imidazolidinyl, pyrazolidinyl, piperidinyl, piperazinyl, azepanyl, diazepanyl (e.g., 1,4-diazepanyl), oxazolidinyl, isoxazolidinyl, thiazolidinyl, isothiazolidinyl, morpholinyl (e.g., morpholin-4-yl), thiomorpholinyl (e.g., thiomorpholin-4-yl), oxazepanyl, oxiranyl, oxetanyl, tetrahydrofuranyl, 1,3-dioxolanyl, tetrahydropyranyl, 1,4-dioxanyl, oxepanyl, thiiranyl, thietanyl, tetrahydrothiophenyl (i.e., thiolanyl), 1,3-dithiolanyl, thianyl, thiepanyl, decahydroquinolinyl, decahydroisoquinolinyl, or 2-oxa-5-aza-bicyclo[2.2.1]hept-5-yl. Unless defined otherwise, “heterocycloalkyl” preferably refers to a 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heterocycloalkyl” include tetrahydropyranyl, piperidinyl, piperazinyl, morpholinyl, pyrrolidinyl, or tetrahydrofuranyl.

As used herein, the term “cycloalkenyl” refers to an unsaturated alicyclic (non-aromatic) hydrocarbon ring group, including monocyclic rings as well as bridged ring, spiro ring and/or fused ring systems (which may be composed, e.g., of two or three rings; such as, e.g., a fused ring system composed of two or three fused rings), wherein said hydrocarbon ring group comprises one or more (e.g., one or two) carbon-to-carbon double bonds and does not comprise any carbon-to-carbon triple bond. “Cycloalkenyl” may, e.g., refer to cyclopropenyl, cyclobutenyl, cyclopentenyl, cyclohexenyl, cyclohexadienyl, cycloheptenyl, or cycloheptadienyl. Unless defined otherwise, “cycloalkenyl” preferably refers to a C₃₋₁₁ cycloalkenyl, and more preferably refers to a C₃₋₇ cycloalkenyl. A particularly preferred “cycloalkenyl” is a monocyclic unsaturated alicyclic hydrocarbon ring having 3 to 7 ring members and containing one or more (e.g., one or two; preferably one) carbon-to-carbon double bonds.

As used herein, the term “heterocycloalkenyl” refers to an unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkenyl” may, e.g., refer to imidazolinyl (e.g., 2-imidazolinyl (i.e., 4,5-dihydro-1H-imidazolyl), 3-imidazolinyl, or 4-imidazolinyl), tetrahydropyridinyl (e.g., 1,2,3,6-tetrahydropyridinyl), dihydropyridinyl (e.g., 1,2-dihydropyridinyl or 2,3-dihydropyridinyl), pyranyl (e.g., 2H-pyranyl or 4H-pyranyl), thiopyranyl (e.g., 2H-thiopyranyl or 4H-thiopyranyl), dihydropyranyl, dihydrofuranyl, dihydropyrazolyl, dihydropyrazinyl, dihydroisoindolyl, octahydroquinolinyl (e.g., 1,2,3,4,4a,5,6,7-octahydroquinolinyl), or octahydroisoquinolinyl (e.g., 1,2,3,4,5,6,7,8-octahydroisoquinolinyl). Unless defined otherwise, “heterocycloalkenyl” preferably refers to a 3 to 11 membered unsaturated alicyclic ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms; more preferably, “heterocycloalkenyl” refers to a 5 to 7 membered monocyclic unsaturated non-aromatic ring group containing one or more (e.g., one, two, or three) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms.

As used herein, the term “halogen” refers to fluoro (—F), chloro (—Cl), bromo (—Br), or iodo (—I).

As used herein, the term “haloalkyl” refers to an alkyl group substituted with one or more (preferably 1 to 6, more preferably 1 to 3) halogen atoms which are selected independently from fluoro, chloro, bromo and iodo, and are preferably all fluoro atoms. It will be understood that the maximum number of halogen atoms is limited by the number of available attachment sites and, thus, depends on the number of carbon atoms comprised in the alkyl moiety of the haloalkyl group. “Haloalkyl” may, e.g., refer to —CF₃, —CHF₂, —CH₂F, —CF₂—CH₃, —CH₂—CF₃, —CH₂—CHF₂, —CH₂—CF₂—CH₃, —CH₂—CF₂—CF₃, or —CH(CF₃)₂. A particularly preferred “haloalkyl” group is —CF₃.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety. Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted.

A skilled person will appreciate that the substituent groups comprised in the compounds of the present invention may be attached to the remainder of the respective compound via a number of different positions of the corresponding specific substituent group. Unless defined otherwise, the preferred attachment positions for the various specific substituent groups are as illustrated in the corresponding exemplary compounds described herein.

As used herein, the term “cccDNA inhibitor” or “HBV cccDNA inhibitor” refers to a compound that is capable of inhibiting hepatitis B-viral (HBV) covalently closed circular DNA (cccDNA), e.g., by inhibiting the stability and/or the transcriptional activity of HBV cccDNA. A cccDNA inhibitor that destabilizes HBV cccDNA, leading to a full or at least partial degradation of the cccDNA, can also be termed a “cccDNA destabilizer” or an “HBV cccDNA destabilizer”, while a cccDNA inhibitor that silences cccDNA transcriptional activity (e.g., via epigenetic mechanisms), without necessarily inducing the degradation of existing HBV cccDNA, can also be referred to as a “cccDNA silencer” or an “HBV cccDNA silencer”. The present invention encompasses any such cccDNA inhibitors, including compounds acting as HBV cccDNA destabilizers and/or silencers, and particularly relates to HBV cccDNA destabilizers. The capability of a compound to destabilize cccDNA can be assessed, e.g., using the cccDNA assay described in Example 1.

As used herein, unless explicitly indicated otherwise or contradicted by context, the terms “a”, “an” and “the” are used interchangeably with “one or more” and “at least one”. Thus, for example, a composition comprising “a” compound of formula (I) can be interpreted as referring to a composition comprising “one or more” compounds of formula (I).

As used herein, the term “about” preferably refers to ±10% of the indicated numerical value, more preferably to ±5% of the indicated numerical value, and in particular to the exact numerical value indicated. If the term “about” is used in connection with the endpoints of a range, it preferably refers to the range from the lower endpoint −10% of its indicated numerical value to the upper endpoint +10% of its indicated numerical value, more preferably to the range from of the lower endpoint −5% to the upper endpoint +5%, and even more preferably to the range defined by the exact numerical values of the lower endpoint and the upper endpoint. If the term “about” is used in connection with the endpoint of an open-ended range, it preferably refers to the corresponding range starting from the lower endpoint −10% or from the upper endpoint +10%, more preferably to the range starting from the lower endpoint −5% or from the upper endpoint +5%, and even more preferably to the open-ended range defined by the exact numerical value of the corresponding endpoint.

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

The scope of the present invention embraces all pharmaceutically acceptable salt forms of the compounds of formula (I) which may be formed, e.g., by protonation of an atom carrying an electron lone pair which is susceptible to protonation, such as an amino group, with an inorganic or organic acid, or as a salt of an acid group (such as a carboxylic acid group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts. Preferred pharmaceutically acceptable salts of the compounds of formula (I) include a hydrochloride salt, a hydrobromide salt, a mesylate salt, a sulfate salt, a tartrate salt, a fumarate salt, an acetate salt, a citrate salt, and a phosphate salt. A particularly preferred pharmaceutically acceptable salt of the compound of formula (I) is a hydrochloride salt. Accordingly, it is preferred that the compound of formula (I), including any one of the specific compounds of formula (I) described herein, is in the form of a hydrochloride salt, a hydrobromide salt, a mesylate salt, a sulfate salt, a tartrate salt, a fumarate salt, an acetate salt, a citrate salt, or a phosphate salt, and it is particularly preferred that the compound of formula (I) is in the form of a hydrochloride salt.

The compounds of formula (I) or their pharmaceutically acceptable salts may also be present in solvated form (i.e., as a solvate). The scope of the invention thus also embraces the compounds of formula (I) or their pharmaceutically acceptable salts in any solvated form, including, e.g., solvates with water (i.e., as a hydrate) or solvates with organic solvents such as, e.g., methanol, ethanol or acetonitrile (i.e., as a methanolate, ethanolate or acetonitrilate). The invention likewise embraces the compounds of formula (I) or their pharmaceutically acceptable salts in any physical form, particularly in any solid form, including in amorphous form or in any crystalline form.

Furthermore, the compounds of formula (I) may exist in the form of different isomers, in particular stereoisomers (including, e.g., geometric isomers (or cis/trans isomers), enantiomers and diastereomers) or tautomers (including, in particular, prototropic tautomers). All such isomers of the compounds of formula (I) are contemplated as being part of the present invention, either in admixture or in pure or substantially pure form. As for stereoisomers, the invention embraces the isolated optical isomers of the compounds according to the invention as well as any mixtures thereof (including, in particular, racemic mixtures/racemates). The racemates can be resolved by physical methods, such as, e.g., fractional crystallization, separation or crystallization of diastereomeric derivatives, or separation by chiral column chromatography. The individual optical isomers can also be obtained from the racemates via salt formation with an optically active acid followed by crystallization. The present invention further encompasses any tautomers of the compounds provided herein.

The scope of the invention also embraces compounds of formula (I), in which one or more atoms are replaced by a specific isotope of the corresponding atom. For example, the invention encompasses compounds of formula (I), in which one or more hydrogen atoms (or, e.g., all hydrogen atoms) are replaced by deuterium atoms (i.e., ²H; also referred to as “D”). Accordingly, the invention also embraces compounds of formula (I) which are enriched in deuterium. Naturally occurring hydrogen is an isotopic mixture comprising about 99.98 mol-% hydrogen-1 (¹H) and about 0.0156 mol-% deuterium (²H or D). The content of deuterium in one or more hydrogen positions in the compounds of formula (I) can be increased using deuteration techniques known in the art. For example, a compound of formula (I) or a reactant or precursor to be used in the synthesis of the compound of formula (I) can be subjected to an H/D exchange reaction using, e.g., heavy water (D₂O). Further suitable deuteration techniques are described in: Atzrodt J et al., Bioorg Med Chem, 20(18), 5658-5667, 2012; William J S et al., Journal of Labelled Compounds and Radiopharmaceuticals, 53(11-12), 635-644, 2010; or Modvig A et al., J Org Chem, 79, 5861-5868, 2014. The content of deuterium can be determined, e.g., using mass spectrometry or NMR spectroscopy. Unless specifically indicated otherwise, it is preferred that the compound of formula (I) is not enriched in deuterium. Accordingly, the presence of naturally occurring hydrogen atoms or ¹H hydrogen atoms in the compounds of formula (I) is preferred. In general, it is preferred that none of the atoms in the compounds of formula (I) are replaced by specific isotopes.

The compounds provided herein may be administered as compounds per se or may be formulated as medicaments. The medicaments/pharmaceutical compositions may optionally comprise one or more pharmaceutically acceptable excipients, such as carriers, diluents, fillers, disintegrants, lubricating agents, binders, colorants, pigments, stabilizers, preservatives, antioxidants, and/or solubility enhancers.

The pharmaceutical compositions may comprise one or more solubility enhancers, such as, e.g., poly(ethylene glycol), including poly(ethylene glycol) having a molecular weight in the range of about 200 to about 5,000 Da (e.g., PEG 200, PEG 300, PEG 400, or PEG 600), ethylene glycol, propylene glycol, glycerol, a non-ionic surfactant, tyloxapol, polysorbate 80, macrogol-15-hydroxystearate (e.g., Kolliphor® HS 15, CAS 70142-34-6), a phospholipid, lecithin, dimyristoyl phosphatidycholine, dipalmitoyl phosphatidycholine, distearoyl phosphatidycholine, a cyclodextrin, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, a carboxyalkyl thioether, hydroxypropyl methylcellulose, hydroxypropylcellulose, polyvinylpyrrolidone, a vinyl acetate copolymer, vinyl pyrrolidone, sodium lauryl sulfate, dioctyl sodium sulfosuccinate, or any combination thereof.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for any desired route of administration, preferably for oral administration. Dosage forms for oral administration include, e.g., coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets.

While the compounds of formula (I) or the above described pharmaceutical compositions comprising a compound of formula (I) may be administered to a subject by any convenient route of administration, it is preferred that they are administered orally (particularly by ingestion/swallowing).

Thus, the compounds or pharmaceutical compositions can be administered orally, e.g., in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropykcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

The compounds or pharmaceutical compositions according to the present invention may be administered to a subject/patient either prior to or after the onset of an HBV infection, preferably after the onset of an HBV infection. Furthermore, several divided dosages, as well as staggered dosages may be administered daily or sequentially. Further, the dosages of the pharmaceutical compositions or formulations may be proportionally increased or decreased as indicated by the exigencies of the therapeutic or prophylactic situation.

Administration of the compounds or pharmaceutical compositions of the present invention to a subject/patient (preferably a human) may be carried out using known procedures, at dosages and for periods of time effective to treat an HBV infection in the subject/patient. An effective amount of the therapeutic compound necessary to achieve a therapeutic effect may vary according to factors such as the state of the disease or disorder in the patient; the age, sex, and weight of the patient; and the ability of the therapeutic compound to treat HBV infection in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a compound of formula (I) according to the invention is from about 1 to about 5000 mg/kg of body weight per day. A person skilled in the art can readily study the relevant factors and make the determination regarding the effective amount of the compound without undue experimentation.

Actual dosage levels of the active ingredients in the pharmaceutical compositions of the invention may be varied so as to obtain an amount of the active ingredient that is effective to achieve the desired therapeutic response for a particular patient, composition, and mode of administration, without being toxic to the patient. In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts. A medical doctor, e.g. a physician, having ordinary skill in the art may readily determine and prescribe the effective amount of the compound or pharmaceutical composition required. For example, the physician may start doses of the compounds of the invention employed in the pharmaceutical composition at levels lower than that required in order to achieve the desired therapeutic effect and gradually increase the dosage until the desired effect is achieved.

In particular, it is advantageous to formulate the compound in dosage unit form for ease of administration and uniformity of dosage. Dosage unit form, as used herein, refers to physically discrete units suited as unitary dosages for the subjects/patients to be treated. Each unit containing a predetermined quantity of therapeutic compound is calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of HBV infection in a patient.

For example, the compounds or pharmaceutical compositions of the invention may be administered to a subject/patient in dosages that range from one to five times per day or more. Alternatively, the compounds or pharmaceutical compositions of the invention may be administered to the subject/patient in a range of dosages that include, but are not limited to, once every day, every two days, every three days to once a week, and once every two weeks. It will be readily apparent to a person skilled in the art that the frequency of administration of the various combination compositions of the invention will vary from individual to individual depending on many factors including, but not limited to, age, disease state, gender, overall health, and other factors. Thus, the invention should not be construed to be limited to any particular dosage regime. The precise dosage and pharmaceutical composition to be administered to any patient will be determined by the attending physician or veterinarian, taking all factors about the patient into account.

The compounds or pharmaceutical compositions of the present invention may be administered orally, e.g., in a dose (referring to the dose of the respective compound of formula (I) in non-salt form) in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, or any whole or partial increment therebetween.

As explained above, the therapeutically effective amount or dose of a compound of the present invention will depend on the age, sex and weight of the subject/patient, the current medical condition of the subject/patient and the progression of HBV infection in the subject/patient to be treated. The skilled person can determine appropriate dosages depending on these and other factors. A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day.

The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

Once improvement of the subject/patient's condition has occurred, a maintenance dose can be administered if necessary. Subsequently, the dosage or the frequency of administration, or both, can be reduced, as a function of the viral load, to a level at which the improved disease is retained. In one embodiment, subjects/patients require intermittent treatment on a long-term basis upon any recurrence of symptoms and/or infection.

The compounds of the invention may be formulated in unit dosage form. The term “unit dosage form” refers to physically discrete units suitable as unitary dosage for subjects/patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of CC₅₀ (the cytotoxicity concentration of compound that cause death to 50% of viable cells) and the IC50 (the minimum concentration to inhibit 50% of the pathogen). The dose ratio between the toxic and therapeutic effects is the therapeutic index, which is expressed as the ratio between CC₅₀ and IC₅₀. Compounds exhibiting high therapeutic indices are preferred. The data obtained from cell culture assays and animal studies are optionally used in formulating a range of dosage for use in human subjects/patients. The dosage of such compounds lies preferably within a range of circulating concentrations that include the IC₅₀ with minimal toxicity. The dosage optionally varies within this range depending upon the dosage form employed and the route of administration utilized.

The compound of formula (I) or a pharmaceutical composition comprising the compound of formula (I) can be administered in monotherapy (e.g., without concomitantly administering any further therapeutic agents against HBV infection). However, the compound of formula (I) or a pharmaceutical composition comprising the compound of formula (I) can also be administered in combination with one or more further therapeutic agents, particularly with one or more further anti-HBV agents (i.e., one or more further therapeutic agents against HBV infection).

Such further anti-HBV agent(s) may be, for example, an HBV polymerase inhibitor, a reverse transcriptase inhibitor, a viral entry inhibitor, a viral maturation inhibitor, a capsid assembly inhibitor/modulator, a TLR agonist, an HBV vaccine, an immunomodulatory agent, an interferon, or a pegylated interferon. Examples of a reverse transcriptase inhibitor (or an HBV polymerase inhibitor) include, in particular, zidovudine, didanosine, zalcitabine, 2′,3′-dideoxyadenosine (ddA), stavudine, lamivudine, abacavir, emtricitabine, entecavir, apricitabine, atevirapine, ribavirin, acyclovir, famciclovir, valacyclovir, ganciclovir, valganciclovir, tenofovir, adefovir, cidofovir, efavirenz, nevirapine, delavirdine, etravirine, telbivudine, or a pharmaceutically acceptable salt, ester or prodrug of any one of the aforementioned agents (such as, e.g., tenofovir alafenamide, tenofovir alafenamide fumarate, tenofovir disoproxil, tenofovir disoproxil fumarate, or adefovir dipivoxil). Examples of a capsid assembly inhibitor/modulator include, in particular, BAY 41-4109 or a pharmaceutically acceptable salt, ester or prodrug thereof. Examples of a TLR agonist include, in particular, a TLR7 agonist or a TLR9 agonist; the TLR7 agonist may be, e.g., SM360320 (or 9-benzyl-8-hydroxy-2-(2-methoxy-ethoxy)adenine), AZD 8848 (or methyl [3-({[3-(6-amino-2-butoxy-8-oxo-7,8-dihydro-9H-purin-9-yl)propyl][3-(4-morpholinyl)propyl]amino}methyl)phenyl]acetate), or a pharmaceutically acceptable salt, ester or prodrug thereof. Examples of an interferon include, in particular, an interferon alpha (e.g., interferon alfa-2a or interferon alfa-2b), an interferon gamma, or an interferon lambda. Examples of a pegylated interferon include, in particular, a pegylated interferon alpha (e.g., peginterferon alfa-2a or peginterferon alfa-2b), a pegylated interferon gamma, or a pegylated interferon lambda. Further examples anti-HBV agent include, without limitation, AT-61 (or (E)-N-(1-chloro-3-oxo-1-phenyl-3-(piperidin-1-yl)prop-1-en-2-yl)benzamide), AT-130 (or (E)-N(1-bromo-1-(2-methoxyphenyl)-3-oxo-3-(piperidin-1-yl)prop-1-en-2-yl)-4-nitrobenzamide), or a pharmaceutically acceptable salt, ester or prodrug thereof.

If the compound of formula (I) is used in combination with a further anti-HBV agent, the dose of each compound may differ from that when the corresponding compound is used alone, in particular, a lower dose of each compound may be used. The combination of the compound of formula (I) with one or more further anti-HBV agents may comprise the simultaneous/concomitant administration of the compound of formula (I) and the further anti-HBV agent(s), either in a single pharmaceutical formulation or in separate pharmaceutical formulations, or the sequential/separate administration of the compound of formula (I) and the further anti-HBV agent(s). If administration is sequential, either the compound of formula (I) according to the invention or the one or more further anti-HBV agents may be administered first. If administration is simultaneous, the one or more further anti-HBV agents may be included in the same pharmaceutical composition/formulation as the compound of formula (I) (particularly as a fixed-dose combination), or they may be administered in two or more different (separate) pharmaceutical compositions/formulations (or different dosage forms). Such different pharmaceutical compositions/formulations may be administered via the same route of administration or via different routes of administration (e.g., one of the agents may be administered orally, and another agent may be administered parenterally). The dosage form of each of the different pharmaceutical compositions/formulations can be suitably chosen depending on the intended route of administration. The two (or more) different pharmaceutical compositions/formulations (or different dosage forms) can also be included in the same packaging, particularly in a combination pack (or convenience pack). Accordingly, as explained above, the compound(s) of formula (I) and the one or more further anti-HBV agents may be provided, e.g., as a fixed-dose combination (i.e., in the same pharmaceutical composition) or as a combination pack (i.e., in separate pharmaceutical compositions which are included in the same packaging).

The present invention thus relates to a compound of formula (I) or a pharmaceutically acceptable salt thereof, or a pharmaceutical composition comprising said compound in combination with a pharmaceutically acceptable excipient, for use in the treatment of an HBV infection (including any of the specific types of HBV infection described herein above), wherein the compound or the pharmaceutical composition is to be administered in combination with one or more further anti-HBV agents (e.g., one or more of the specific anti-HBV agents described above, such as an interferon alpha (e.g., interferon alfa-2a or interferon alfa-2b), an interferon gamma, an interferon lambda, pegylated interferon alpha (e.g., peginterferon alfa-2a or peginterferon alfa-2b), a pegylated interferon gamma, a pegylated interferon lambda, zidovudine, didanosine, zalcitabine, 2′,3′-dideoxyadenosine (ddA), stavudine, lamivudine, abacavir, emtricitabine, entecavir, apricitabine, atevirapine, ribavirin, acyclovir, famciclovir, valacyclovir, ganciclovir, valganciclovir, tenofovir, tenofovir alafenamide, tenofovir alafenamide fumarate, tenofovir disoproxil, tenofovir disoproxil fumarate, adefovir, adefovir dipivoxil, cidofovir, efavirenz, nevirapine, delavirdine, etravirine, telbivudine, BAY 41-4109, SM360320, AZD 8848, AT-61, AT-130, or a pharmaceutically acceptable salt, ester or prodrug of any one of the aforementioned agents). The combined administration of the compound or the pharmaceutical composition of the present invention with one or more further anti-HBV agents may be effected, e.g., by simultaneous/concomitant administration (either in a single pharmaceutical formulation or in separate pharmaceutical formulations) or by sequential/separate administration.

The subject or patient to be treated in accordance with the present invention may be an animal (e.g., a non-human animal). Preferably, the subject/patient is a mammal. More preferably, the subject/patient is a human (e.g., a male human or a female human) or a non-human mammal (such as, e.g., a woodchuck, a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, or a pig). Most preferably, the subject/patient to be treated in accordance with the invention is a human. The subject/patient (which/who is preferably a human subject) may further be, for example, an immunocompromised subject, an HIV-positive subject, an immunosuppressed subject, or an organ transplant recipient.

As used herein, and unless contradicted by context, the term “treatment” or “treating” (of a disease or disorder) refers to curing, alleviating, reducing or preventing one or more symptoms or clinically relevant manifestations of a disease or disorder, or to alleviating, reversing or eliminating the disease or disorder, or to preventing the onset of the disease or disorder, or to preventing, reducing or delaying the progression of the disease or disorder. For example, the “treatment” of a subject or patient in whom no symptoms or clinically relevant manifestations of the respective disease or disorder have been identified is a preventive or prophylactic treatment, whereas the “treatment” of a subject or patient in whom symptoms or clinically relevant manifestations of the respective disease or disorder have been identified may be, e.g., a curative or palliative treatment. Each one of these forms of treatment may be considered as a distinct aspect of the present invention.

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease), palliative treatment (including symptomatic relief), or prophylactic treatment (including prevention) of the disorder or disease.

It is to be understood that the present invention specifically relates to each and every combination of features and embodiments described herein, including any combination of general and/or preferred features/embodiments. In particular, the invention specifically relates to each combination of meanings (including general and/or preferred meanings) for the various groups and variables comprised in formula (I).

It is further to be understood that all steps of any method described herein can, in general, be performed in any suitable order, unless indicated otherwise or contradicted by context. Preferably, any such method steps are carried out in the specific order in which they are indicated.

In this specification, a number of documents including patents or patent applications, scientific literature and manufacturers' manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The reference in this specification to any prior publication (or information derived therefrom) is not and should not be taken as an acknowledgment or admission or any form of suggestion that the corresponding prior publication (or the information derived therefrom) forms part of the common general knowledge in the technical field to which the present specification relates.

The invention is also described by the following illustrative figures. The appended figures show:

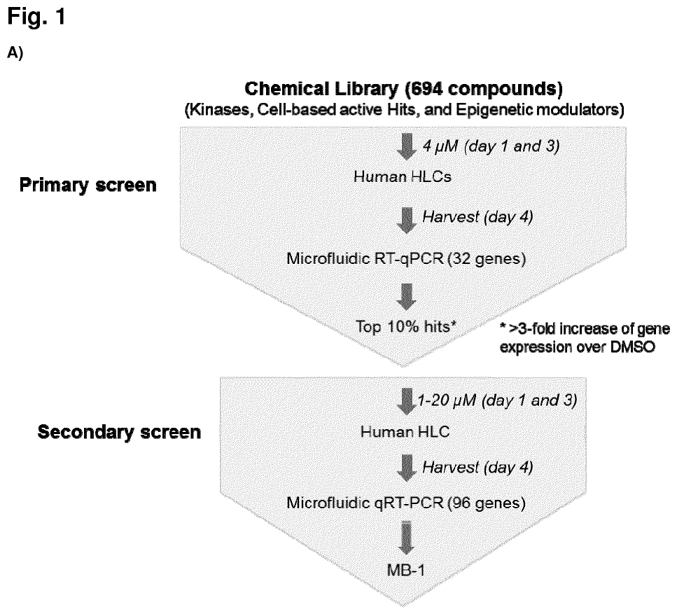

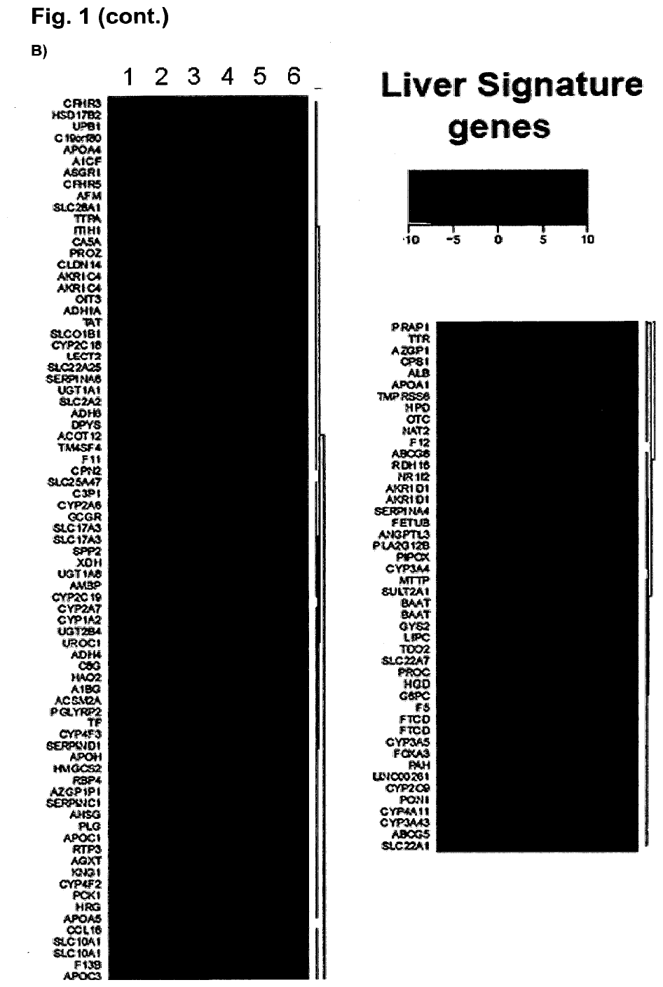

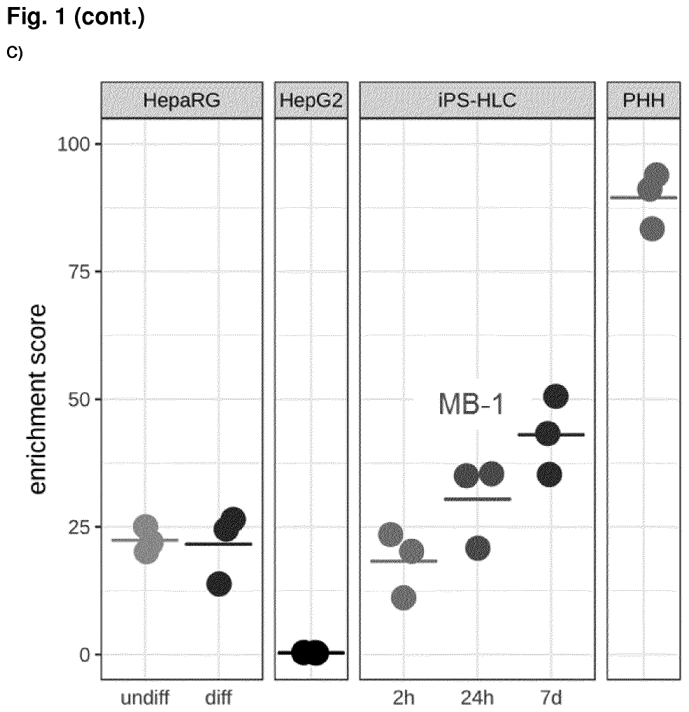

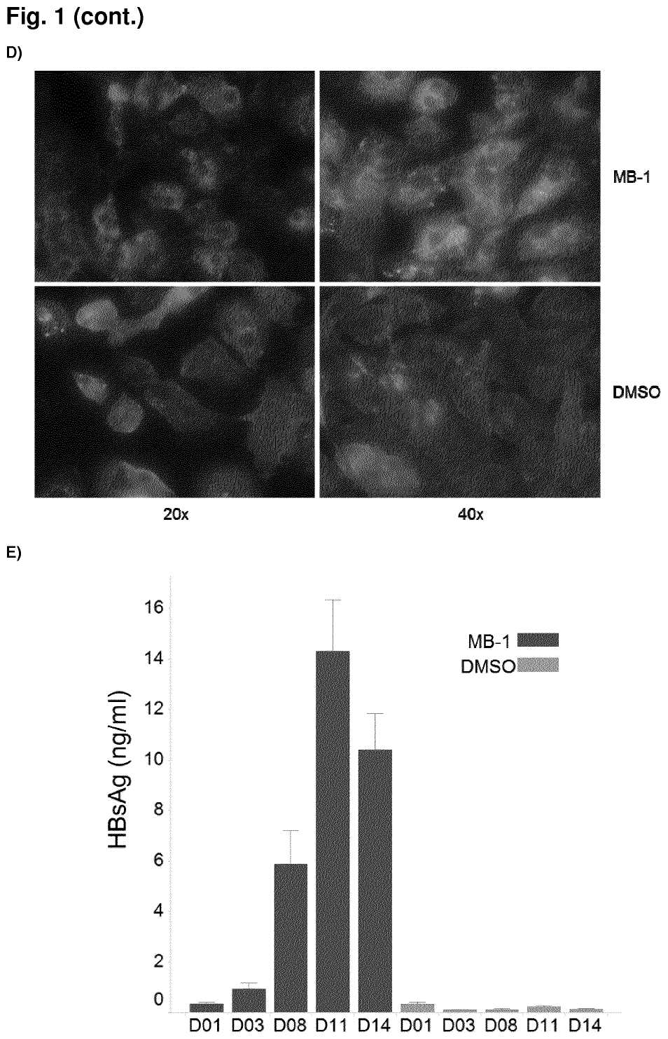

FIG. 1: Maturation screen identified a small molecule that enhances maturation of HLC. (A) HLC maturation screening cascade. (B) A whole-genome transcriptome microarray was performed on HLC following treatment with MB-1 and mRNA expression of 137 liver signature genes (those that are highly expressed in the liver with specificity index gini >0.8) is shown. Lane 1-3, MB-1 (5 μM) at 7 d, 24 h, and 2 h, respectively. Lane 4-6, MB-1 (0.5 μM) at 7 d, 24 h, and 2 h, respectively. (C) BioQC liver score analysis of HLC transcriptomes (following treatment with MB-1 for 2 h, 24 h, and 7 day) were compared to those of PHH, HepaRG, and HepG2. (D) Expression of AAT-1a in HLC after 14 day of treatment with MB-1, or 1% DMSO. (E) MB-1-treated HLC support robust HBV infection: HLC (in 96-well plate) were treated with MB-1 (1 μM), or 1% DMSO, for 4 days, then infected with patient-derived HBV (MOI 10, in triplicate). Culture medium was harvested at indicated time points and analyzed for HBsAg.

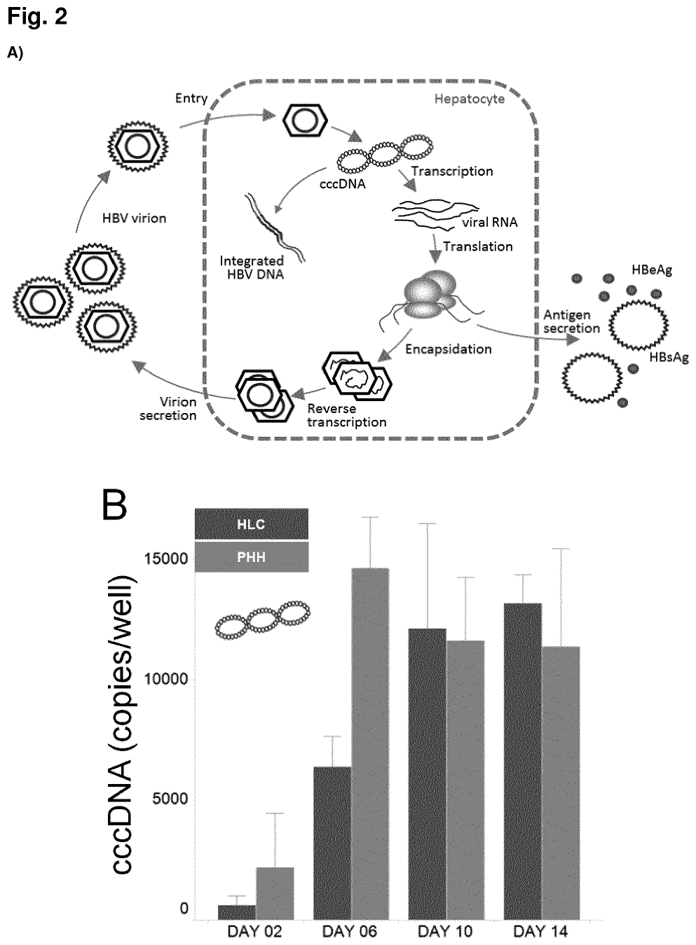

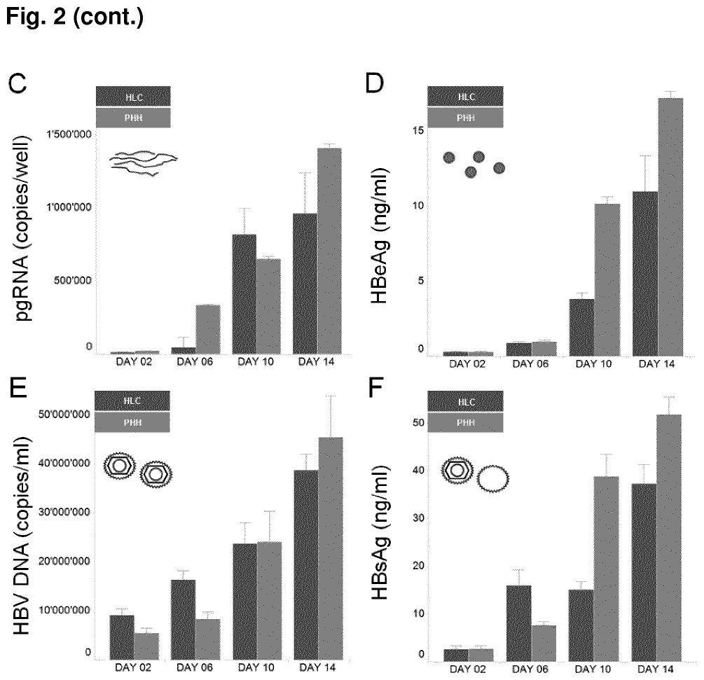

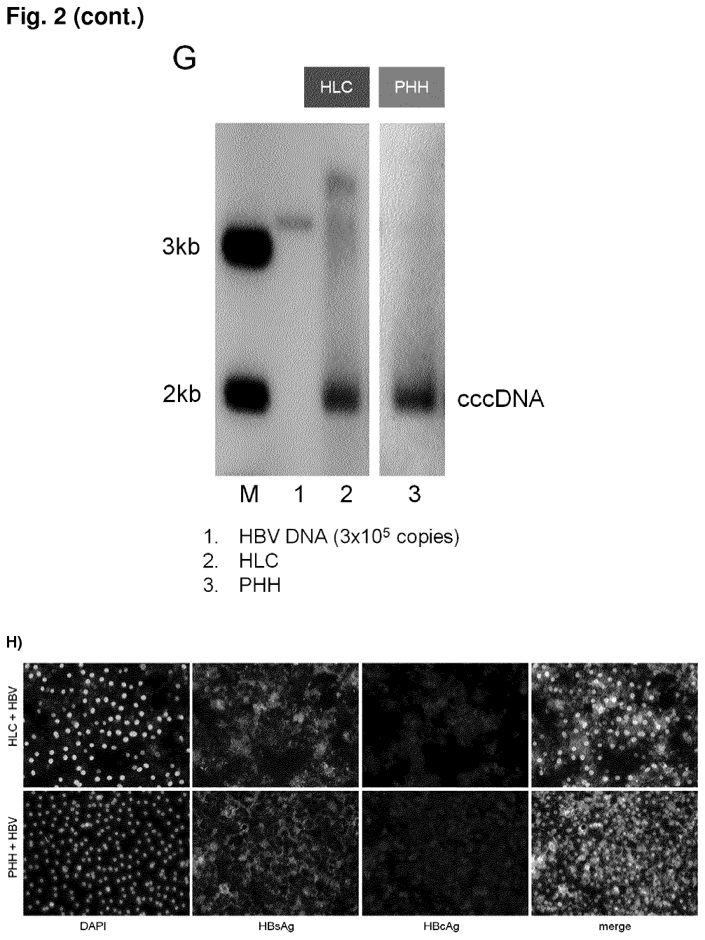

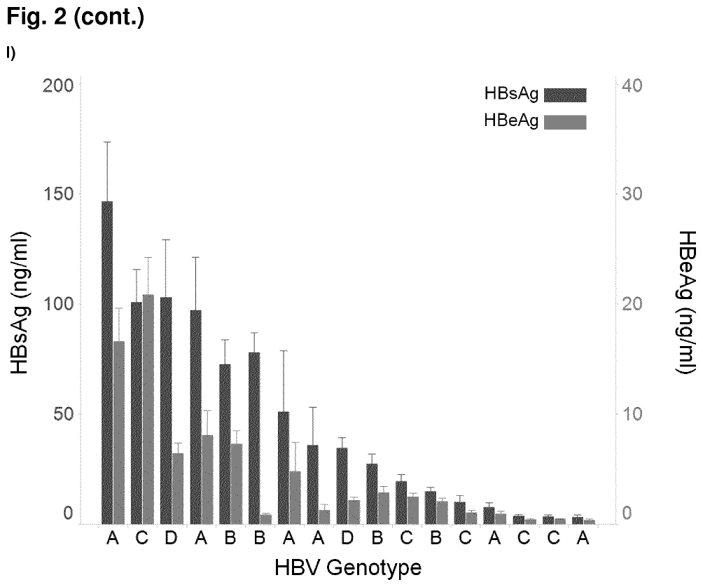

FIG. 2: HLC is a disease-relevant model for HBV. (A) Schematic of HBV life cycle. (B-F) Kinetics of HBV infection in HLC and PHH: Cells (in 96-well plate) were infected with HBV (MOI 40, in triplicate), and cultured for 14 day. Culture media and cell lysates were harvested at the indicated time points and assayed for various HBV markers as shown. (G) Detection of cccDNA in HBV-infected cells by Southern Blot assay: HLC and PHH were infected with patient-derived HBV and harvested at day 10 pi by Hirt extraction method. Samples were digested with T5 exonuclease before loaded on the gel. A full-length, 3.2 kb HBV (+) strand RNA probe was used for HBV DNA detection. (H) Immunostaining of HLC- and PHH-infected cells with anti-HBs and anti-HBc antibodies. (I) HLC support robust infection of clinical HBV isolates from various GTs (MOI 40 in triplicate, 384-well plate). HBsAg and HBeAg were measured at day 14 pi.

FIG. 3: A ˜247K HTS in HLC to discover novel cccDNA inhibitors. (A) Reproducibility of HLC and PHH assays: Cells were infected with patient-derived HBV at MOI 40. At day 3 pi, a reference compound was added at 3-fold dilution starting from 100 μM. Experiments were repeated 30 times in HLC and 62 times in PHH; each line represents HBsAg or HBeAg IC50 curve for each experiment. (B) Schematic of HTS assay (in HLC) and screening cascade (in PHH). (C) HLC primary hits: A stacked dot plot graph of HLC primary hits based on multiplex readout (compounds that inhibited albumin >40% were excluded from analysis). Each dot in the graph showed a compound that either inhibits HBsAg (blue), or HBeAg (green). The dotted red box highlights 3752 compounds that inhibit both HBV antigens >60%. (D) Potency of HLC hits in PHH (n=1027). HLC hits were tested in PHH, and their IC50 values against HBsAg and HBeAg in HLC and PHH is shown. Dotted lines indicate the average HBsAg and HBeAg IC50 values of all compounds in each cell type. (E) Correlation between HBsAg/HBeAg and pgRNA activity of HLC hits in HLC and PHH: HLC hits (n=244) showed good correlation between their potency against HBsAg and HBeAg (median IC50 values 1.72 μM and 1.55 μM, respectively) vs pgRNA activity (median IC50 2.64 μM). Similar results were obtained in PHH with 127 compounds showed median HBsAg, HBeAg, and pgRNA IC50 values of 11.40 μM, 10.90 μM, and 12.20 μM, respectively. (F) Confirmation of activity of cccDNA destabilizers in PHH by Southern Blot assay. PHH were infected with HBV (GT D), and at day 3 pi, treated with compound 7 and reference compound 1 at 2 or 6 μM. At day 10 pi, Hirt extracts were prepared and analyzed by Southern Blot. Mitochondrial DNA (mtDNA) was used as a loading control for each sample.

FIG. 4: Molecular phenotyping of cccDNA destabilizers in PHH. (A) Principal Component Analysis (PCA): Three day after infection with HBV (or treated with 1% DMSO), PHH were incubated with compound 7 and reference compound 1 (each with its less active isomer) for 6 hr, then harvested. All experimental conditions were performed in triplicate. PCA shown was based on AmpliSeq-RNA data of 917 pathway reporter genes. (B) Pathway heat map of cccDNA destabilizers: Pathways that are significantly (p<0.001) regulated by HBV, or by either compound 7 or reference compound 1, are visualized in the heat map.

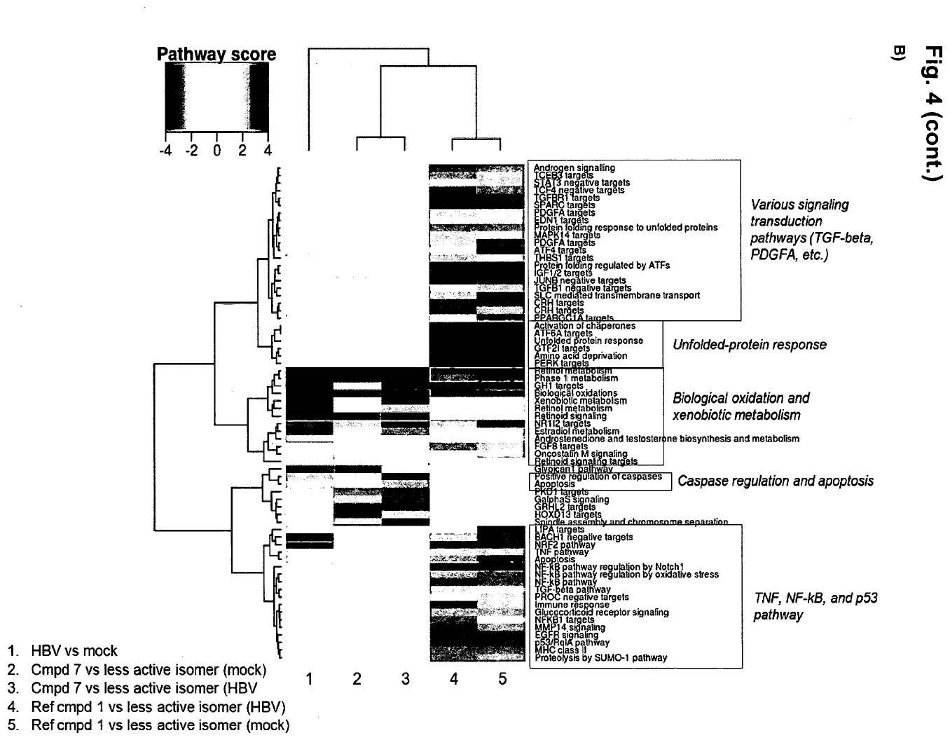

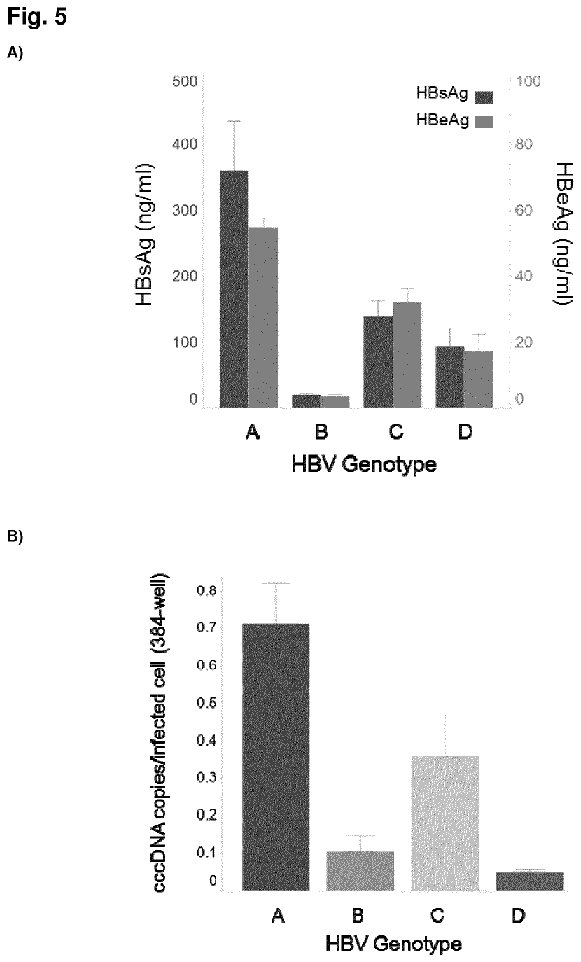

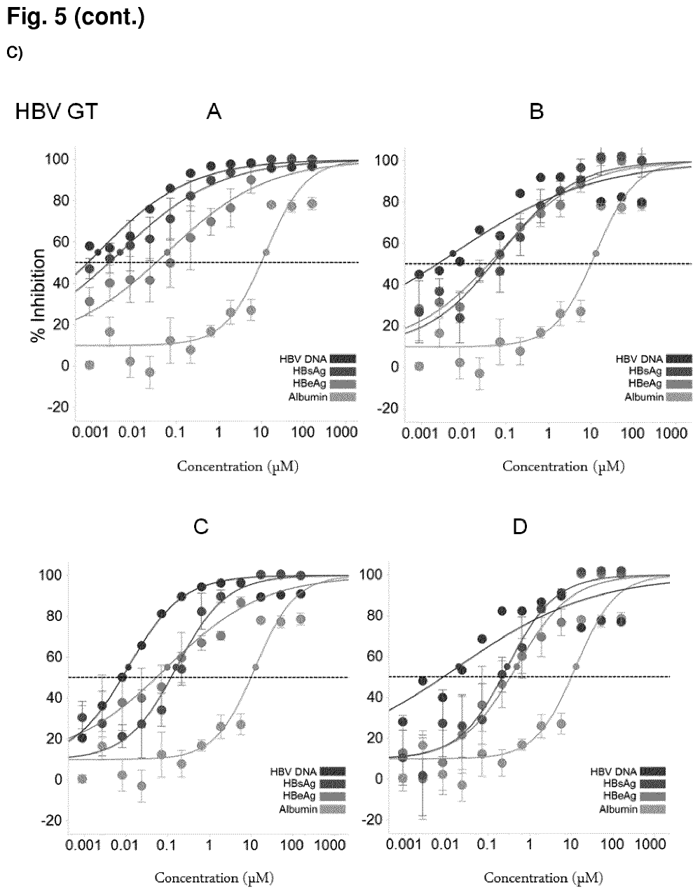

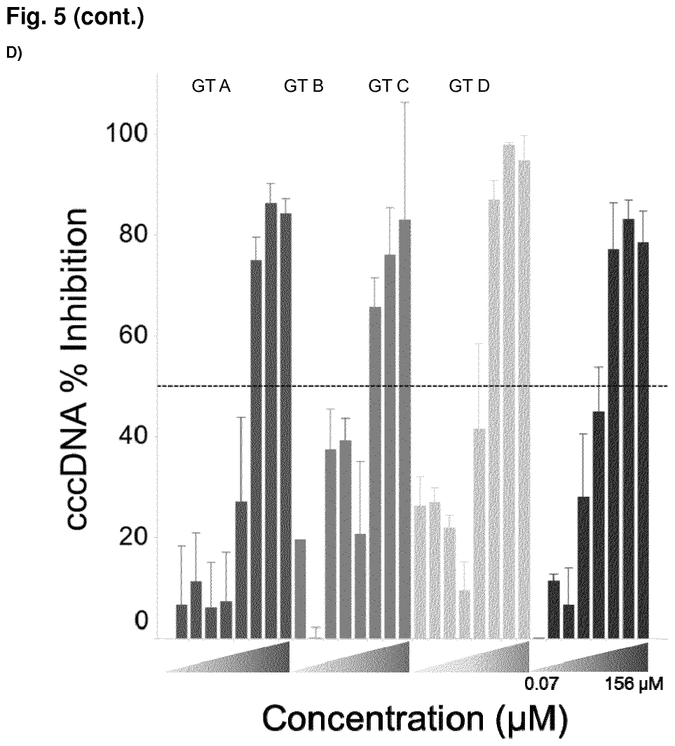

FIG. 5: Antiviral activity of compound 7 against patient-derived HBV GT A-D in PHH. PHH seeded in 384-well plate were infected with patient-derived HBV (GT A-D) at MOI 40 in triplicate. At day 3 pi, compound 7 was added in 3-fold dilutions; starting at 156 μM. 1% DMSO was used as negative control. Fresh medium and compound was replenished every 2 day and cells were harvested at day 10 pi. (A-B) Baseline levels of HBsAg and HBeAg released into culture medium, and cccDNA copy number/well (384-well plate format) of HBV genotypes A-D at day 10 pi in the absence of compound. (C) Antiviral activity of compound 7 against HBV GT A-D based on HBsAg, HBeAg, and HBV DNA readouts. Albumin is a cellular tox marker. (D) Antiviral activity of compound 7 against HBV GT A-D based on cccDNA readout (digital PCR).

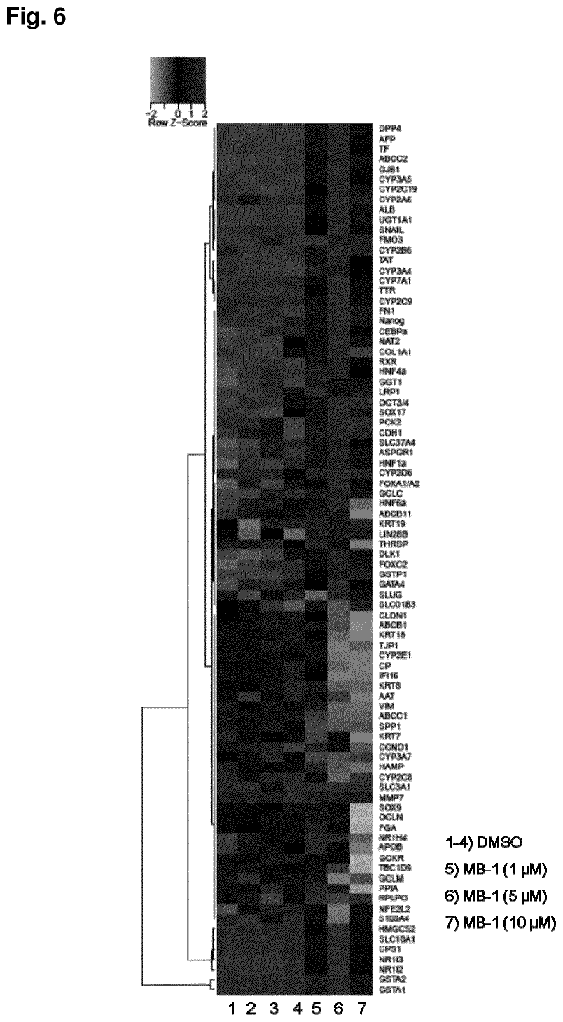

FIG. 6: Effect of MB-1 on the expression of 96 liver-enriched genes in HLC. HLC seeded on collagen I-coated 6-well plates were treated with MB-1 (1, 5, or 10 μM) in 1% DMSO, or 1% DMSO (each in triplicate) for 4 days. Cells were harvested and expression of 96 liver-enriched genes was analyzed by microfluidic RT-qPCR (Fluidigm).

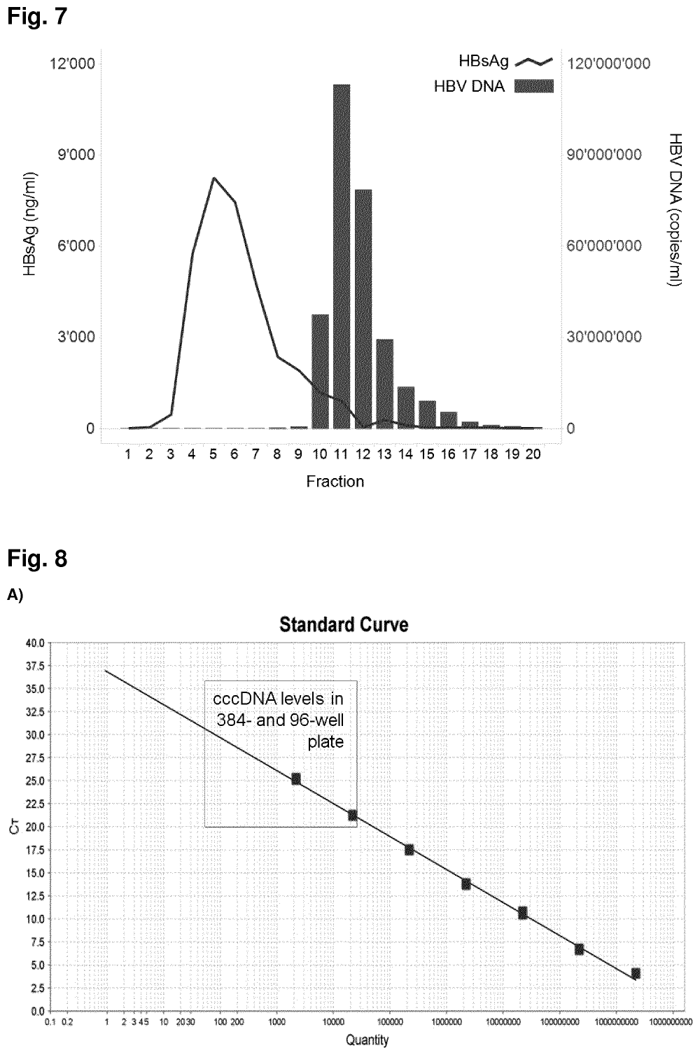

FIG. 7: HBV purification from patient serum on OptiPrep™ gradient. HBV was purified from sera of CHB individual using OptiPrep™ density gradient (100,000×g for 2 hours at 4° C.) in SW41 tubes (BD Biosciences). Twenty fractions (500 μl each) were collected from the top, and aliquots for each fraction were analyzed for HBV DNA and HBsAg. Peak fractions that contain high amount of HBV DNA (virus particles) are pooled and used for infection experiments.

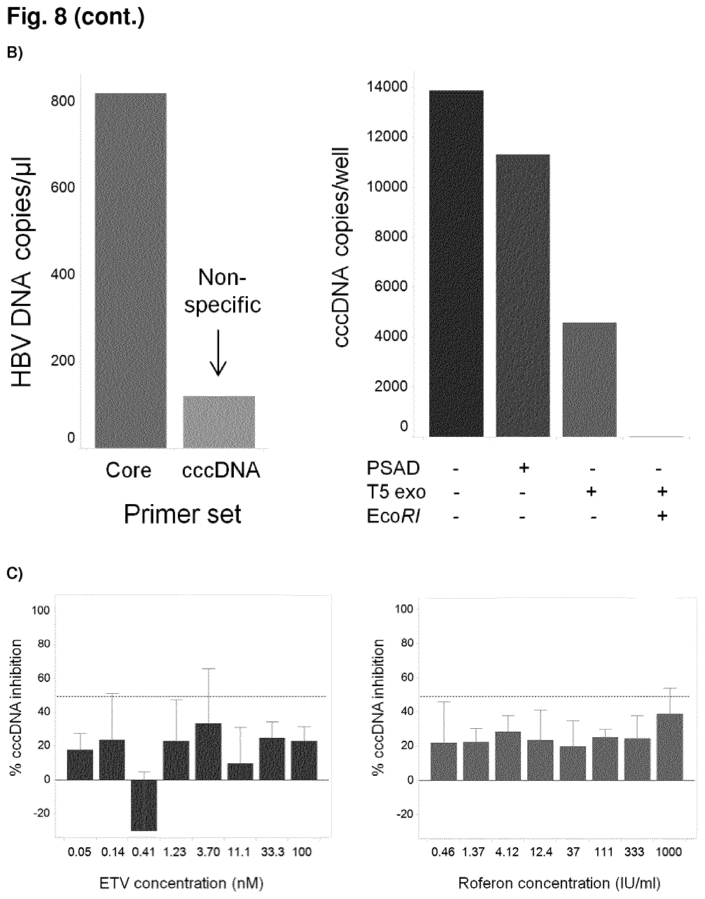

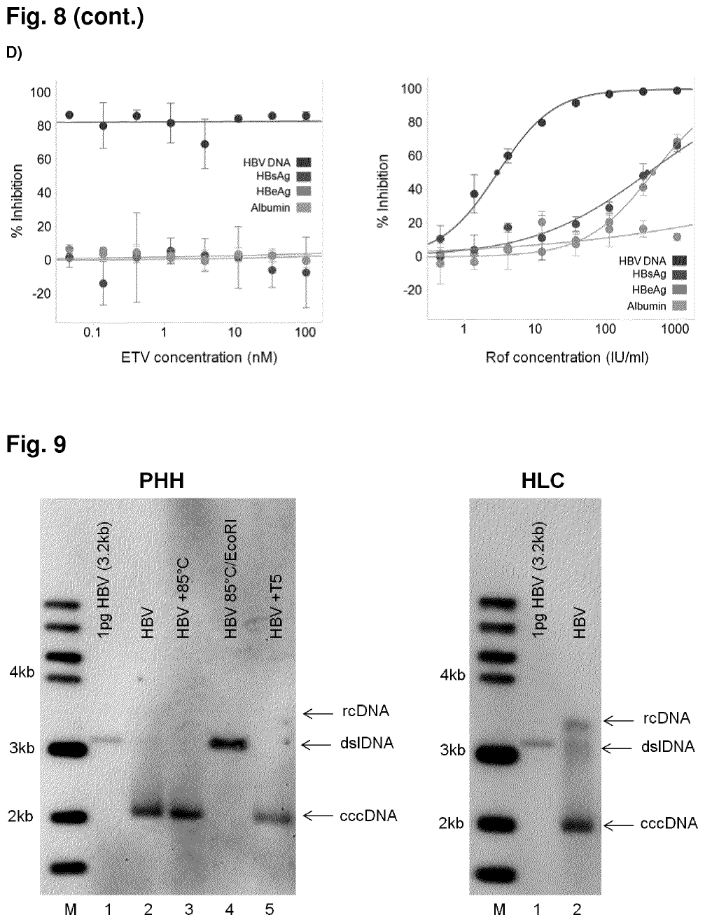

FIG. 8: Establishment of dPCR assay for cccDNA quantification. (A) Detection ranges of TaqMan-PCR assay limits accurate determination of cccDNA copy number from 96- and 384-well plate. A 3.2-kb, linearized plasmid HBV is used as a standard curve for relative quantification of HBV DNA by TaqMan-PCR. Plasmid was diluted 10-fold (from 2×10⁹ copies/μl to 2×10³ copies/μl) and HBV DNA was amplified using core primers (Werle-Lapostolle et al., 2004); the LLOD of this assay (˜1×10³ copies/μl) overlaps with the lower levels of cccDNA present in cells grown in 384-well plate. The total amount of cccDNA in HLC and PHH in 384-well plate is ˜1,200-12,000 copies/well (assuming 40% infection rate of ˜30K cells seeded, and on average, there is 0.1-1 cccDNA copy/cell) (Nassal, 2015). (B) Testing primer & PCR specificity and removal of excess RC-DNA. In contrast to TaqMan-PCR (relative quantification method), digital PCR (dPCR) is an absolute quantification method without the need of standard curve. It also more sensitive (˜50-fold) than TaqMan-PCR; assay precision can be increased by testing more replicates (array/through-holes) per sample. First step—Testing primer and PCR specificity. Serum-derived HBV (contains RC-DNA, devoid of cccDNA) was used as a DNA template for dPCR using 2 sets of primers (for HBV core and cccDNA region) (Werle-Lapostolle et al., 2004). Samples were amplified by dPCR on QuantStudio 12K Flex Real-Time PCR System (AB); 4 subarrays (256 through-holes) were used for each sample. Low signal was detected with cccDNA primer, indicating non-specific amplification of RC-DNA. Second step—Removal of excess RC-DNA. PHH seeded in 96-well were infected with HBV. At day 6 pi, cell lysates were digested with Plasmid safe, ATP-dependent, DNAse (PSAD), T5 exonuclease, or, T5 exonuclease followed by EcoRI, for 1 hr at 37° C. Samples were amplified by dPCR using cccDNA primers on QuantStudio 12K Flex Real-Time PCR System (AB); 4 subarrays (256 through-holes) were used for each sample. Treatment with T5 exonuclease prior to dPCR efficiently removed excess of RC-DNA. (C) Effect of entecavir (ETV) and Roferon on HBV DNA, HBsAg, HBeAg and cccDNA in PHH. To validate the dPCR assay for cccDNA detection in naturally infected cells, PHH were infected with patient-derived HBV (GT D at MOI 40), and 3 days later, were treated with ETV and Roferon at the indicated concentrations. Both compounds are highly potent against HBV DNA, but have no effect on other viral markers. Fresh medium and compound was replenished every 2 day. At day 10 pi, culture media were harvested and measured for HBV DNA, HBsAg, and HBeAg. Albumin was used as a surrogate of in vitro tox marker. Cells were lysed and treated with T5 exonuclease, cccDNA was then measured by dPCR on QuantStudio 12K Flex Real-Time PCR System (AB); 4 subarrays (256 through-holes) were used for each sample. (D) Effect of entecavir (ETV) and Roferon (Rof) on HBV DNA, HBsAg, and HBeAg in PHH. Cells were treated with compounds at the indicated concentrations starting from day 3 post infection (patient-derived HBV, GT D). Fresh medium and compound was replenished every 2 day. Cells were harvested at day 10 pi; viral markers and albumin were measured from culture medium.

FIG. 9: Detection of cccDNA in HBV-infected PHH and HLC by Southern Blot assay. Cells grown in 24-well plate were infected with patient-derived HBV (GT D) and harvested at day 10 pi by Hirt extraction method. (Left, PHH) To verify that the primary band detected in HBV-infected cells (lane 2) is cccDNA, sample was heated at 85° C. for 5 min to denature rcDNA and dslDNA into ssDNA (lane 3), and digested with EcoRI to convert cccDNA into dslDNA (lane 4), or digested with T5 exonuclease to remove any nicked/linear DNA fragment (lane 5). Each lane 2-5 corresponding to ˜1 million cells. A full-length, 3.2 kb HBV (+) strand RNA probe was used for HBV DNA detection. rcDNA, relaxed circular DNA; dslDNA, double-stranded linear DNA; cccDNA, covalently closed circular DNA.

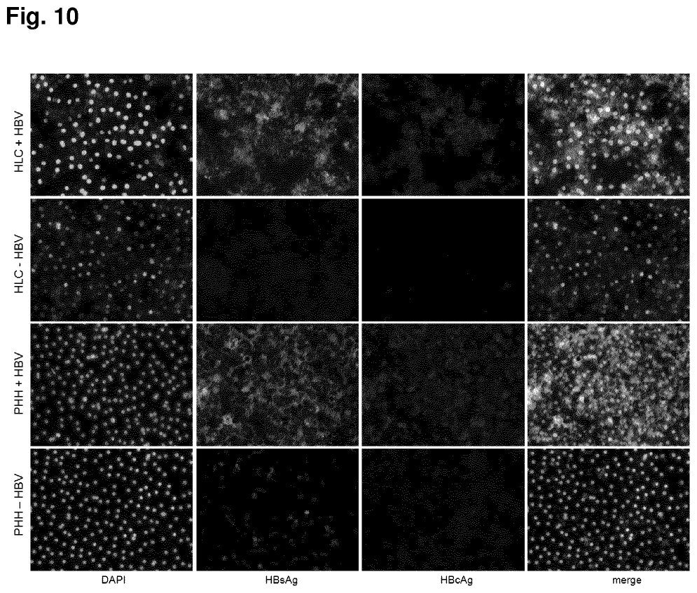

FIG. 10: Immunostaining of HLC and PHH infected with patient-derived HBV (GT A). Cells seeded in 384-well plate were infected with patient-derived HBV (GT A at MOI 40) and at day 12 pi, were fixed and stained with anti-HBV core and anti-HBs antibodies.

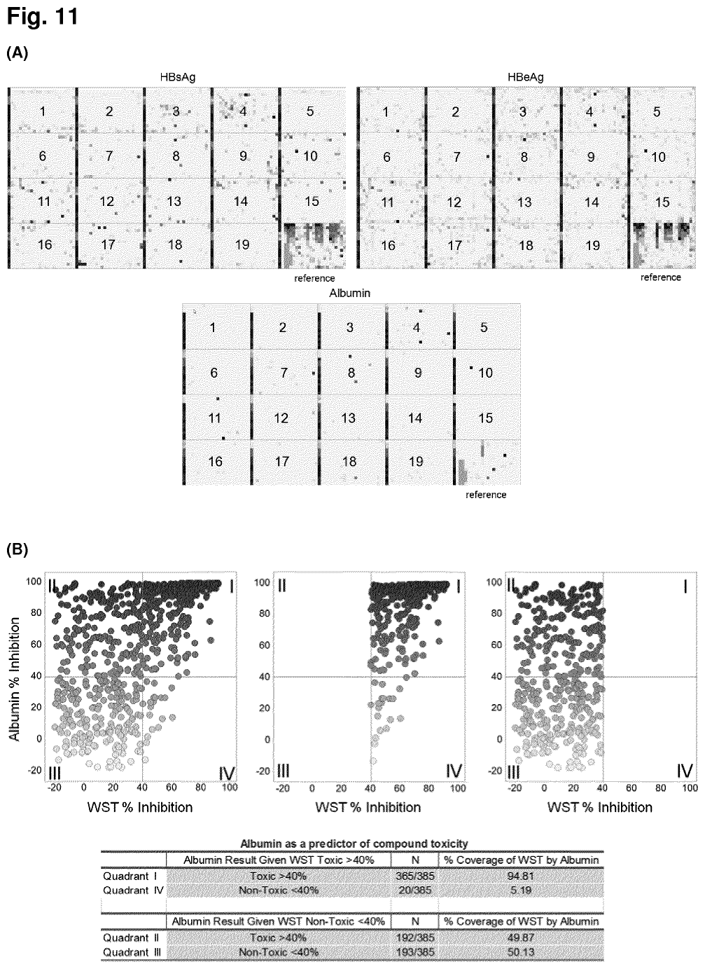

FIG. 11: Multiplex assay as primary HTS readout. (A) Determination of Z-score: HLC seeded in 384-well plates were infected with patient-derived HBV (MOI 40) in the presence of 1% DMSO (19 plates), or treated with reference compounds (1 plate), total 20 plates. At day 14 pi, culture media from all plates were simultaneously measured for HBsAg, HBeAg, and albumin by a Luminex-based, multiplex assay (Radix BioSolutions, Georgetown, Tex.). Data analysis was performed by GeneData software, and images for each analyte on each plate were captured. Numbers indicated each of (384-well) plate. (B) Albumin as a predictor of compound toxicity: HLC seeded in 384-well plates were infected with patient-derived HBV (MOI 40) and were treated with 385 compounds starting from day 3 pi. Fresh medium and compound was replenished every 2 day. At day 14 pi, culture media were harvested and analysed for albumin inhibition by multiplex assay, and cell lysates were analysed by a standard in vitro toxicity assay (Cell Proliferation Reagent/WST-1; cat #11 644 807 001, Roche Diagnostics). Three graphs, each delineated with 4 quadrants, showed that albumin could predict 94.81% of compound toxicity detected by WST-1 (quadrant 1). Importantly, albumin inhibition can be used to filter out non-specific inhibitors (192 compounds, 49.87%) that were not detected by WST-1 (quadrant II).

FIG. 12: Molecular phenotyping (heat map of host pathways affected by nucleoside analog and interferon-α). At 3 day pi, PHH were treated with either nucleoside analog (ETV), or IFN-α, at their 1×IC90 values for 6 h. Total RNA was extracted using RLT buffer (QIAGEN), reverse-transcribed, and the cDNA product was amplified using Ion AmpliSeq™ RNA Library Kit (Life Technologies, Carlsbad, USA, cat #4482335). Pathway analysis was performed using CAMERA method (Wu & Smyth, 2012) and gene sets in an internally available database (RONET) which integrates publicly available gene sets such as MSigDB (Liberzon et al., 2011) and REACTOME (Fabregat et al., 2016). Results of CAMERA are represented by enrichment scores, which are defined by the absolute log 10-transformed p-value returned by CAMERA multiplied by either +1 (positive regulation of the gene set) or −1 (negative regulation of the gene set).

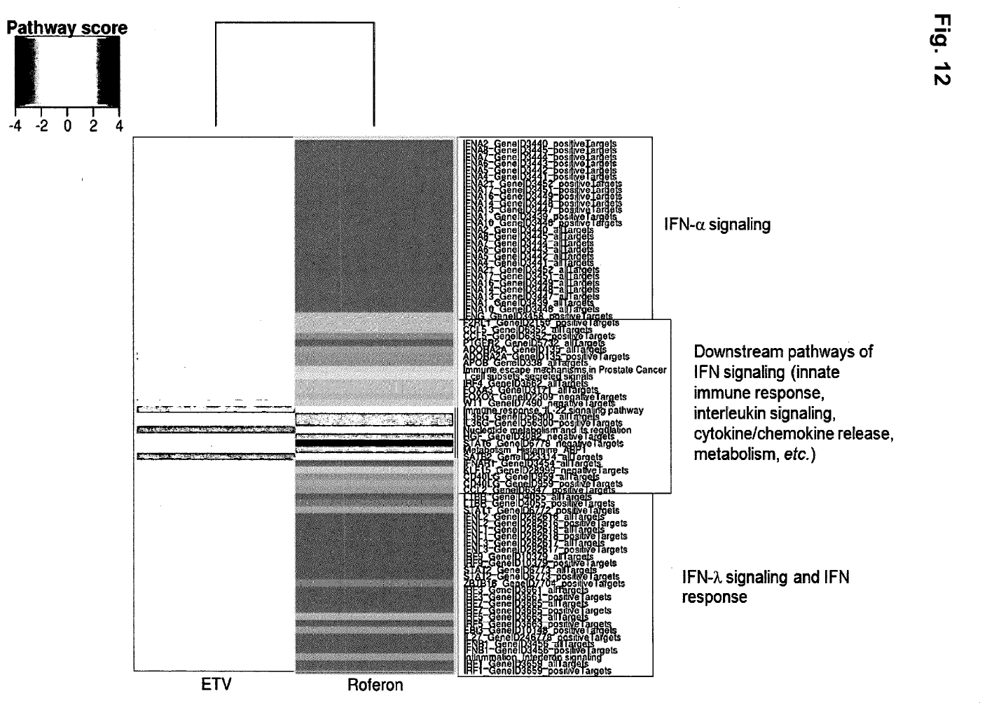

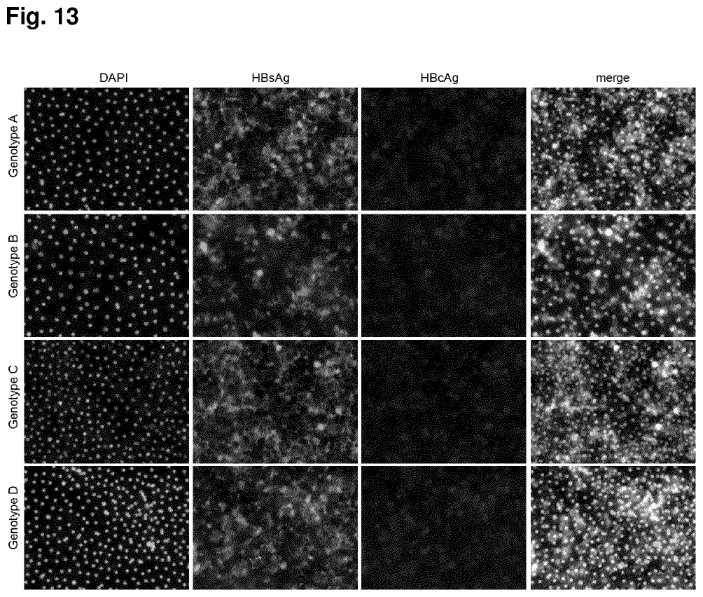

FIG. 13: Pan-genotypic (GT A-D) HBV infection in PHH. Cells were infected with each HBV isolate/GT at MOI 40. Ten days later, immunostaining was performed with anti-HBs and anti-HBc antibodies.

FIG. 14: (A) Screening cascade rationale to increase the likelihood to identify cccDNA inhibitors. (B) Preferred criteria of screening cascade to identify cccDNA inhibitors.

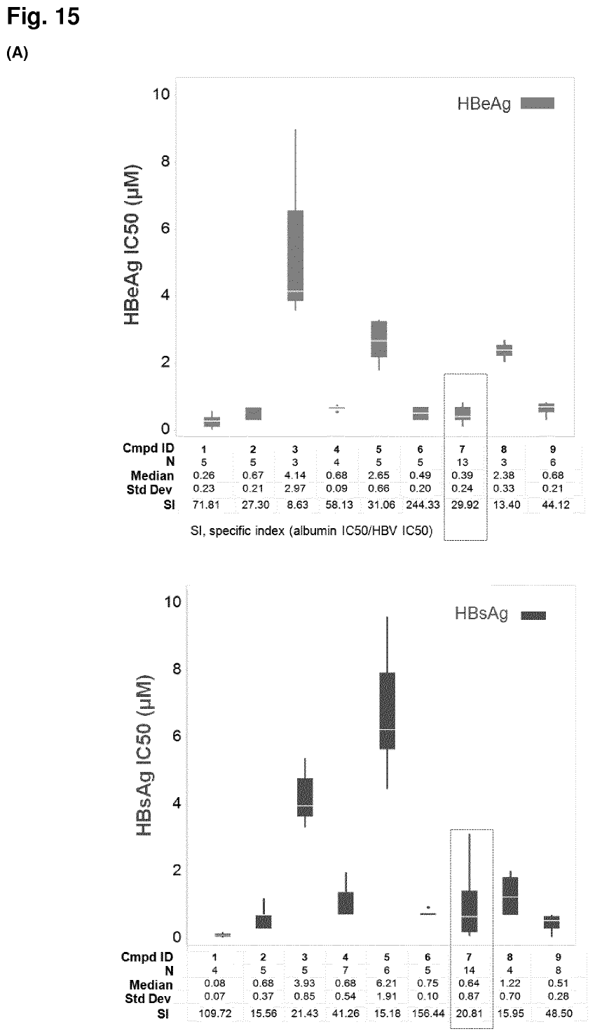

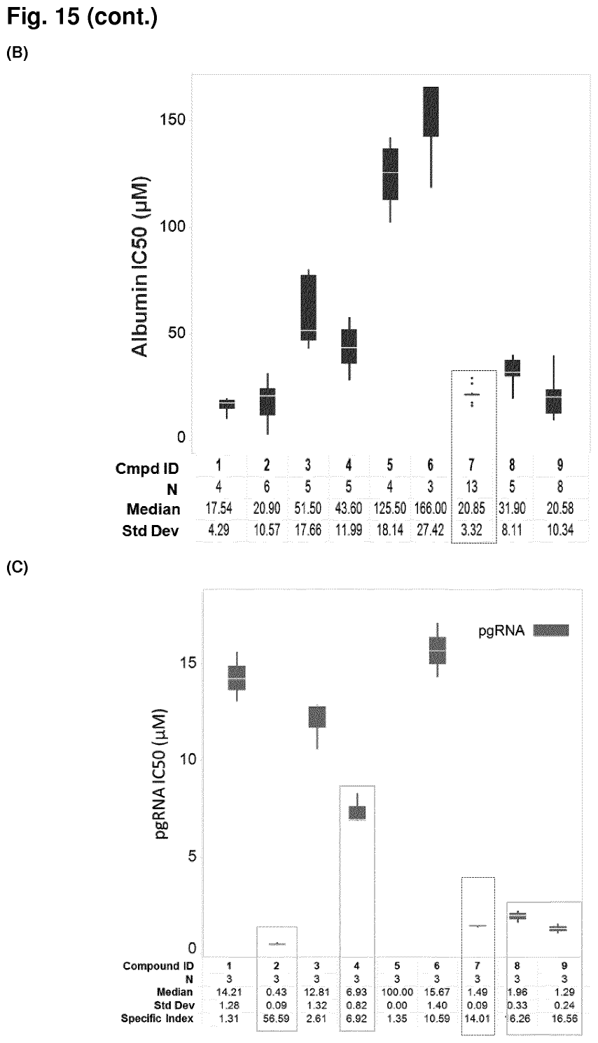

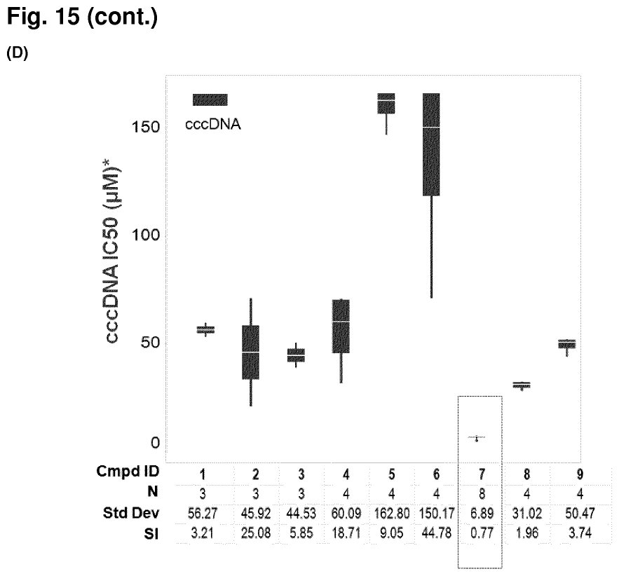

FIG. 15: (A) HBeAg and HBsAg activity, (B) albumin activity, (C) pgRNA activity, and (D) cccDNA activity of pyrrolo[2,3-b]pyrazine compounds in PHH (patient-derived HBV, GT D). See Example 2.

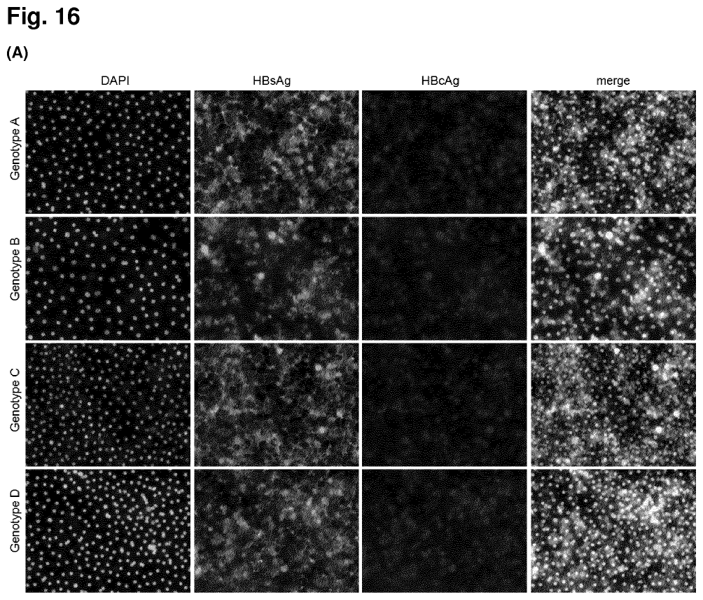

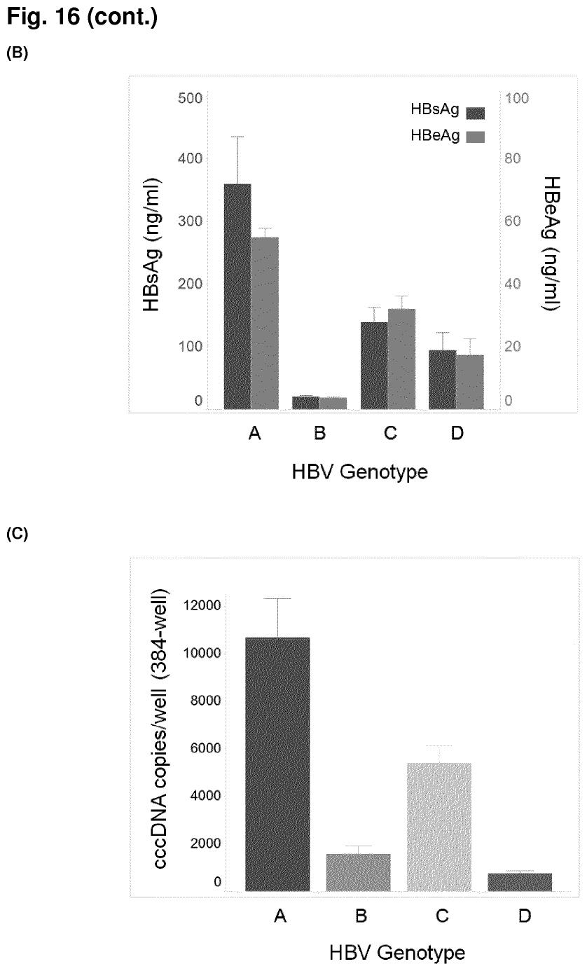

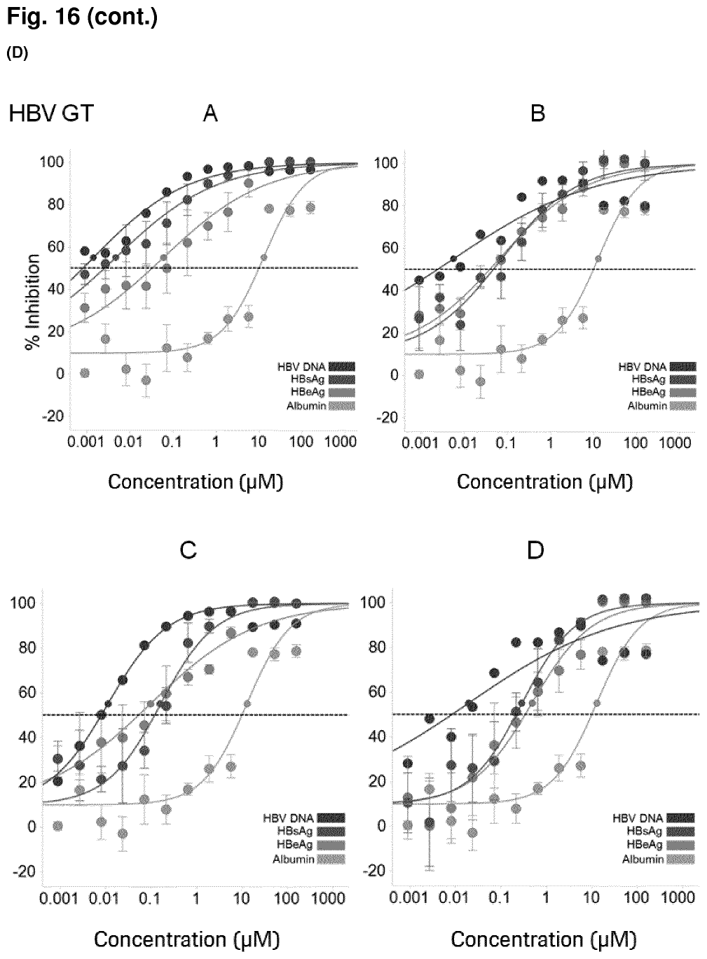

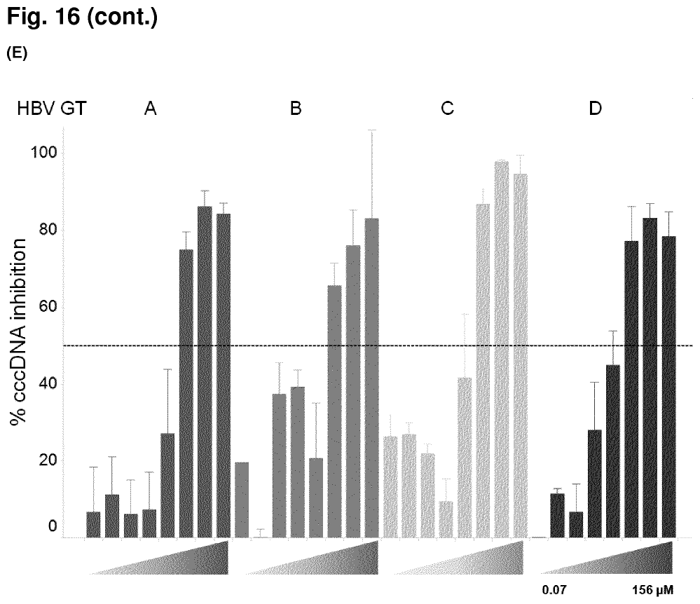

FIG. 16: Antiviral activity of compound 7 against patient-derived HBV GT A-D in PHH (see Example 2). (A) Immunostaining of PHH-infected cells using anti-HBs and anti-HBc antibodies. (B-C) Levels of HBsAg and HBeAg released into culture medium, and cccDNA copy number/well of HBV genotypes A-D at day 10 pi in the absence of compound. (D) Antiviral activity of compound 7 against HBV GT A-D based on HBsAg, HBeAg, and HBV DNA readouts. Albumin is a cellular tox marker. (E) Antiviral activity of compound 7 against HBV GT A-D based on cccDNA readout.

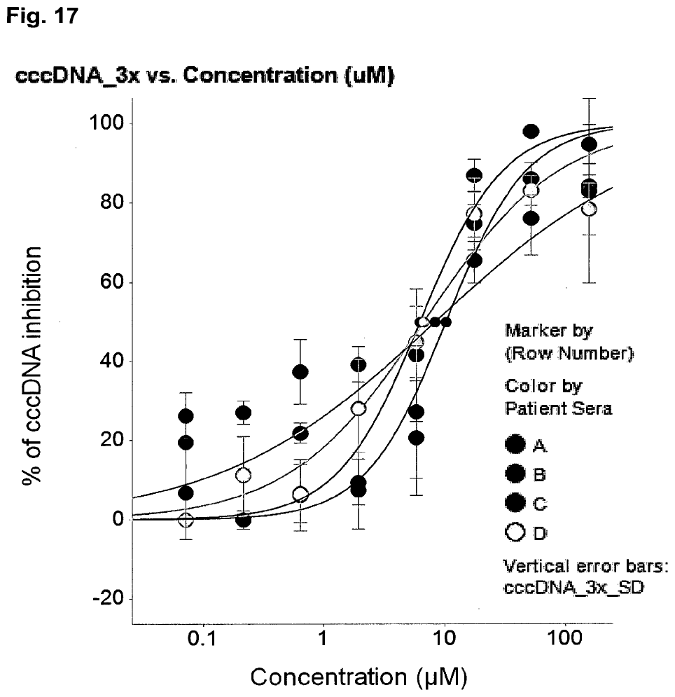

FIG. 17: Pan-GT, cccDNA activity of compound 7 against HBV GT A-D (see Example 2).

The invention will now be described by reference to the following examples which are merely illustrative and are not to be construed as a limitation of the scope of the present invention.

### EXAMPLES

### Example 1: Phenotypic Screening in Stem Cell-Derived Hepatocyte-Like Cells that Recapitulate Complete HBV Life Cycle from Clinical Isolates to Discover cccDNA Inhibitors

### Methods

### Patient Sera Purification

HBV from sera of CHB individuals were purified using OptiPrep™ (Axis-Shield, Norway) density gradient. Briefly, OptiPrep™ stock solution (60%) was diluted to 50% and 10% in PBS; equal volume of each solution was then added into SW41 tubes (BD Biosciences). Linear gradient was performed by placing the tubes on Gradient Master 108™ (Biocomp) at setting: 800, 25 rpm for 30″. Two hundred microliter (200 μl) of serum was overlaid on the top of gradient and samples were centrifuged at 100,000×g for 2 hours at 4° C. Fractions (500 μl) were collected from the top, and aliquots for each fraction were analyzed for HBV DNA using core primers 5′-CTGTGCCTTGGGTGGCTTT (forward), 5′-AAGGAAAGAAGTCAGAAGGCAAAA (reverse), 56-FAM/AGCTCCAAATTCTTTATAAGGGTCGATGTCCATG/31ABlk_FQ/(probe) (Werle-Lapostolle et al., 2004) and HBsAg. Fractions containing the peak of HBV DNA were pooled and used as virus inoculum for all infection experiments. All fractions were stored at −80° C. until used.

iPS-Derived Hepatocyte-Like Cells (HLC)

Cryopreserved HLC were thawed and seeded according to manufacturer's recommendation. Briefly, cryopreserved cells were thawed in a 37° C. water bath for 2 min, and the content of cryovial was poured into the 15 ml tube containing 12 ml of 37° C. iCell Hepatocytes Medium B (KryoThaw Component A 7.8 ml, KryoThaw Component B 4.2 ml). The tube was inverted slowly (˜5 times) then centrifuged at 110×g at room temperature for 10 minutes. After medium was aspirated, 2 ml of RT iCell Hepatocytes Medium C (RPMI containing B27 supplement, Oncostatin M 20 ng/ml, dexamethasone 1 μM, and gentamicin 25 μg/ml) was added, and cells were counted. Cell suspension was then diluted in Medium C containing Matrigel 0.25 mg/ml at 1 million cells/ml. Cells were seeded onto a collagen 1-coated cell culture plate at 40,000 cells/well (384-well plate), or 100,000 cells/well (96-well plate), and cultured at 37° C. incubator in a humidified atmosphere with 5% CO₂. Culture medium was replaced 24 hr post-plating with Medium D (RPMI containing B27 supplement, Oncostatin M 20 ng/ml, dexamethasone 0.1 μM, and gentamicin 25 μg/ml) containing Matrigel 0.25 mg/ml and 1 μM MB-1. Fresh medium and MB-1 was changed every 2 day.

### HLC Maturation Screening

HLC are seeded on collagen 1-coated 96-well plates in 100 μl medium D containing Matrigel 0.25 mg/ml. The next day (day 1), compound library was added to cells at a final concentration 4 μM in 1% DMSO. Fresh medium and compound was replenished 2 days later (day 3). At day 4, cells were harvested using Cells-to-Ct lysis kit (Ambion/Thermo Fisher), total RNAs were reverse-transcribed, and the resulting cDNA products were loaded into the microfluidic 96.96 Dynamic Array™ IFC and assayed against 32 liver-enriched genes on Biomark HD system (Fluidigm). The relative gene expression was calculated from delta Ct values using house-keeping gene (PPIA) in DMSO control as reference; delta Ct values were then converted to fold-change values. Compounds that increased liver-enriched gene expression in HLC ≥3-fold compared to DMSO control were further tested in dose-response (1, 5, 10, and 50 μM). The secondary screen was performed as above using 96-liver enriched genes. The top candidate (MB-1) was used for all experiments utilizing HLC.

### PXB-PHH

Fresh primary human hepatocytes (PXB-PHH) harvested from humanized mice (uPA/SCID mice)—herein called PHH—were obtained from PhoenixBio Co., Ltd (Japan). Cells were seeded on a collagen I-coated plate at the following cell density: 35,000 cells/well (384-well), 70,000 cells/well (96-well), or, 400,000 cells/well (24-well) in modified hepatocyte clonal growth medium (dHCGM). dHCGM is a DMEM medium containing 100 U/ml Penicillin, 100 μg/ml Streptomycin, 20 mM Hopes, 44 mM NaHCO₃, 15 μg/ml L-proline, 0.25 μg/ml insulin, 50 nM Dexamethazone, 5 ng/ml EGF, 0.1 mM Asc-2P, 2% DMSO and 10% FBS (Ishida et al., 2015). Cells were cultured at 37° C. incubator in a humidified atmosphere with 5% CO₂. Culture medium was replaced 24 h post-plating and every 2 days until harvest.

### HBV Infection and Compound Treatment

Following 4 day of maturation with 1 μM MB-1, HLC were incubated with HBV (purified from CHB individuals) at multiplicity of infection (MOI) 10-40 without PEG for 24 hr; virus inoculum was removed the following day. HBV infection in PHH was performed at MOI 40+4% PEG. Compound treatment in HLC and PHH was started at day 3 post infection. Compound (in powder) was dissolved in DMSO; the final concentration of DMSO added to cells is 1%. Fresh compound was replenished every 2 day until cells were harvested at day 10 (PHH), or day 14 (HLC). Compound effect on HBV and cellular toxicity was measured by multiplex assay (HBsAg, HBeAg, albumin), branched DNA (pgRNA), or digital PCR (cccDNA) and depicted as % of inhibition compared to DMSO control. Graphs were prepared using Spotfire software.

### High Throughput Screening (HTS)

HLC seeded in collagen 1-coated 384-well plates were treated with 1 μM MB-1 for 4 day (medium and compound was replenished every 2 day). At day 4, cells were infected with HBV (purified from sera of CHB individuals) at MOI 40 for 24 hr; virus inoculum was removed and fresh medium was added. At day 3 pi, compound library were added at final concentration of 4 μM in 1% DMSO; fresh medium and compound were replenished every 2 day until day 14. Throughout 18-day HTS assay, cells were cultured at 37° C. incubator in a humidified atmosphere with 5% CO₂; all liquid handlings were carried out with robotic equipment in BSL3** facility. At day 14 pi, culture media were harvested and processed for multiplex assay. Approximately 20,000 compounds were screened in each run.

### HTS Readouts

### Multiplex Assay—Primary Readout

A custom, Luminex-based multiplex assay that simultaneously measured HBeAg, albumin, and HBsAg was developed by Radix BioSolutions (Georgetown, Tex.). This is a sandwich immunoassay; each capture antibody was coupled with xMAP™ Luminex magnetic beads. The dynamic ranges of analyte detection are as follows: HBeAg (1-316 ng/ml), albumin (3.1-10,000 ng/ml), and HBsAg (0.1-100 ng/ml) with coefficient variant (CV) 525%. Samples were read on FlexMAP 3D (Luminex) and analyzed by Genedata software. The table below showed that multiplexed beads and detection antibodies against each analyte did not cross-reactive between analytes (numbers reported as mean fluorescence intensity/MFI).

| | Conc. | | | |
|---|---|---|---|---|
| Standard | ng/ml | HBeAg | HBsAg | Albumin |
| HBeAg | 316 | 2901 | 85 | 61 |
| | | 3056 | 88 | 59 |
| HBsAg | 100 | 27 | 6865 | 60 |
| | | 29 | 7349 | 57 |
| Albumin | 10,000 | 27 | 68 | 5143 |
| | | 33 | 70 | 5607 |
| Blank | 0 | 31 | 69 | 63 |
| | | 27 | 65 | 57 |

pgRNA Assay (Branched DNA)—Secondary Readout

Levels of pgRNA in infected cells (96-well plate) were measured using QuantiGene Singleplex 2.0 assay (Affymetrix), a hybridization-based assay that utilizes the xMAP™ Luminex magnetic beads and branched DNA (bDNA) signal amplification technology. The assay is performed in 96-well plate according to manufacturer's recommendation. Briefly, cells were lysed and lysates were incubated with HBV probe sets panel at 50-55° C. for 30 min then stored at −80° C. Signal amplification is achieved via sequential hybridization of PreAmplifier, Amplifier, and Label Probe. After adding Streptavidin phycoerythrin (SAPE) substrate, the signal is read using a FlexMap 3D (Luminex) instrument.

cccDNA Assay (Digital PCR)—Third Readout

HBV-infected cells (in 384- or 96-well plate) were lysed with Cells-to-CT Lysis Reagents according to manufacturer's instruction (Thermo Scientific). To remove excess of RC-DNA, samples were digested with T5 exonuclease (10 U) (New England Biolabs), at 37° C. for 1 hr; enzyme was inactivated by heating the samples at 80° C. for 15 min. DNA samples (1.2 μl) were added into the digital PCR Master Mix (QuantStudio Digital PCR Kit, Thermo Scientific) containing cccDNA primers 5′-CTCCCCGTCTGTGCCTTCT (forward), 5′-GCCCCAAAGCCACCCAAG (reverse), and CGTCGCATGGAGACCACCGTGAACGCC (probe) (Werle-Lapostolle et al., 2004) in a total volume 5 μl, and loaded into dPCR array using AccuFill System (AB). Each sample was loaded into 4 subarrays/256 through-holes. Digital PCR assay was run on QuantStudio 12K Flex (AB) and data was analyzed by Digital Suite Software (AB).

cccDNA Assay (Southern Blot)—Confirmation/Fourth Readout

HLC or PHH were seeded in 12-well plate format and infected with HBV as described above. At day 10, HIRT extracts were prepared from cells as follows. Briefly, 500 μl HIRT lysis buffer was added to each well and lysates from three wells were combined to isolate protein-free HBV DNA following the standard HIRT extraction procedure (Cai et al., 2013). For Southern blot analysis, 0.2 μl of Quick-Load 1-kb DNA ladder (New England Biolabs), 2 μg of a 1×HBV genome-length (3.2 kb) PCR product (Primer P1/P2, Guenther et al., 1995), and 5 μg of HIRT-extracted DNA were loaded per lane and separated by electrophoresis in a 1.0% (wt/vol) agarose gel in 1× Tris-Acetate-EDTA buffer at 50V for 3.5 h. After electrophoresis, the DNA was depurinated, denatured, and neutralized as described (Cai et al., 2013) then transferred onto Hybond XL membrane (Amersham) using TurboBlotter system (GE Healthcare). HBV DNA was detected with a DIG-labeled (+) strand HBV RNA probe transcribed from a 1×HBV genome-length (3.2 kb) PCR product with T7 Promoter (HBV T7+Forward Primer 5′-TAATACGACTCACTATAGGGTTTTCACCTCTGCCTAATCATC-3′, HBV Reverse Primer 5′-CCTCTAGAGCGGCCGCAAAAAGTTGCATGGTGCTGGT-3′) using the DIG Northern Starter Kit (Roche) according to manufacturer's instructions. Mitochondrial DNA was detected with an RNA probe binding to the ND1 gene region of the mitochondrial genome (Ducluzeau et al., 1999). Hybridization, washes, and detection with CDP-Star (Roche) were carried out according to manufacturer's instructions. Images were acquired with a FUSION Fx (Vilber) and bands quantified by densitometry using the FUSION-CAPT software.

Immunostaining Immunostaining was performed using Image-iT™ Fixation/Permeabilization kit (Thermo Fisher, cat #R37602). At day 10 pi, cells were fixed in 1 ml of Fixative solution for 15 min at room temperature (RT), then washed three times with 2 ml of Wash buffer for 2-5 min. Cells were incubated with primary and subsequently, secondary, antibodies diluted in D-PBS buffer containing 3% BSA, fraction V, de-lipidated, New Zealand source, each for 1 hr at RT. Primary antibodies: anti-HBs mAb, MAK_M_RF18 (Roche) at 1.25 μg/mL, or, anti-HBV core antibody at 0.1 μg/mL (DAKO, Cat no. B0586). Secondary antibodies: goat anti-rabbit IgG (H+L) cross-adsorbed secondary antibody, Alexa Fluor 594 (Thermo Fisher cat no. A-11012), or, goat anti-mouse IgG (H+L) cross-adsorbed secondary antibody, Alexa Fluor 488 (Thermo Fisher cat no. A-11001) at 2 μg/ml. After three washes with 2 ml Wash buffer for 2-5 min, cells were incubated with Hoechst 33342, Trihydrochloride, Trihydrate (Thermo Fisher cat no. H3570) at 1 μg/ml for 15 min at RT. Immunostaining was analyzed with Axio Observer inverted microscope (Zeiss) and Zeiss ZEN Software.

### Molecular Phenotyping

At day 3 pi, cells were treated with compound or 1% DMSO for 6 hr. Total RNA was extracted using RLT buffer (QIAGEN, Hombrechtikon, Switzerland) and samples were stored at −80° C. Ten (10) ng of total RNA from each biological replicate was reverse-transcribed; the cDNA product was amplified according to the protocol supplied with the Ion AmpliSeq™ RNA Library Kit (Life Technologies, Carlsbad, USA, Catalog number 4482335). After primer digestion, adapters and barcodes were ligated to the amplicons followed by magnetic bead purification. The purified library was amplified, purified and stored at −20° C. Amplicon size and DNA concentration was measured using an Agilent High Sensitivity DNA Kit (Agilent Technologies, Waldbronn, Germany) according to the manufacturer's guide. Pathway analysis was performed with the CAMERA method (Wu & Smyth, 2012) and gene sets in an internally available database (RONET) which integrates publicly available gene sets such as MSigDB (Liberzon et al., 2011) and REACTOME (Fabregat et al., 2016). Results of CAMERA are represented by enrichment scores, which are defined by the absolute log 10-transformed p-value returned by CAMERA multiplied by either +1 (positive regulation of the gene set) or −1 (negative regulation of the gene set).

### Results

Identification of a Small Molecule that Enhanced Maturation of HLC

To fully manifest the potential of HLC as a disease-relevant assay for HBV drug discovery, they have to meet the following criteria: hepatocyte-likeness, scalability, assay robustness, and reproducibility. It is well known that HLC still display immature phenotypes i.e. resemble more fetal than adult, hepatocytes (Baxter et al., 2015; Godoy et al., 2015; Goldring et al., 2017). The difficulties to obtain fully mature HLC with current protocols are multifactorial, including variability of donor origin (Kajiwara et al., 2012; Heslop et al., 2017), and culture conditions that poorly emulate the complexity of liver architecture including liver zonation (Goldring et al., 2017). Indeed, hepatocytes differentially expressed key liver genes and consequently, different metabolic functions, depending on their location along the porto-central axis of the liver (Halpern et al., 2017; Soto-Gutierrez et al., 2017; Torre et al., 2010). Another critical issue for the application of HLC in drug discovery is scalability; billions of cells (with high purity and minimal batch-to-batch variability) are needed to run HTS and subsequent iterative rounds of hit follow up. The inventors chose HLC from a commercial source (CDI, Madison, Wis.) and their first effort was to improve its maturation using a small molecule library consisting of ˜700 biologically active compounds. Cells were cultured according to manufacturer's recommendation (Lu et al., 2016) and incubated with compound library (4 μM). To identify hits that enhanced hepatic maturation, the inventors applied a two-step screening cascade based on up-regulation of 32 (first screen) or 96 (second screen) liver-enriched genes (see FIG. 1A and Table 1). The top hit, MB-1, was chosen based on its ability to enhance mRNA expression of liver-enriched genes at a relatively low concentration (55 μM) (see FIG. 6). A genome-wide microarray analysis showed that MB-1 up-regulated mRNA expression of liver tissue signature i.e. ˜237 liver-enriched genes (see Table 2) in HLC in a time- and dose-dependent manner with 137 genes that are highly expressed in the liver (specificity thresholds: Gini index >0.7 and >0.8, respectively; see Zhang et al., 2017 for the definition of Gini index) (see FIG. 1B). Among those are HBV-dependency factors such as SLC10A1 (NTCP, the HBV receptor), and the transcription factors HNF4α, RXRα, and PPAR, that are essential for HBV pregenomic RNA synthesis and viral DNA replication (Tang & McLachlan, 2001). The inventors compared liver tissue signature of HLC to those of other HBV systems (HepG2, HepaRG and PHH) using BioQC analysis. BioQC is supervised bioinformatics software that enables comparison of any gene expression data against 150 tissue-enriched gene signatures; results are reported as enrichment scores of each tissue signature for each sample in the form of log 10p (absolute log 10-transformed p-value of Wilcoxon test) (Zhang et al., 2017). At baseline, HLC has a comparable liver score to HepaRG; MB-1 treatment considerably increased HLC's liver score even higher than that of HepaRG (see FIG. 1C). MB-1 is not sufficient to further differentiate HLC into adult hepatocytes, which could be attributed to monolayer culture conditions along with other, unknown factors. Both HLC and HepaRG also showed more liver-like phenotypes than HepG2. The poor similarity of HepG2 cell line to PHH is known, many of the liver-enriched genes are either down-regulated or completely “turned off” in HepG2 (Uhlen et al., 2015). Effect of MB-1 on hepatic maturation of HLC was also observed at the protein level; HLC expressed higher level of hepatocyte-specific AAT1α protein (see FIG. 1D).

TABLE 1
List of liver-enriched genes used for primary (32) and secondary (96) screens in Hepatic Maturation Screen

| First screen (32 genes) | | Second screen (96 genes) | | | | | |
|---|---|---|---|---|---|---|---|
| Gene | | Gene | | Gene | | Gene | |
| Symbol | Description | Symbol | Description | Symbol | Description | Symbol | Description |
| SLC10A1 | Bile Acid Synthesis | ABCB1 | Transporters | CYP2C9 | Phase 1 metabolizing | FN1 | EMT mesenchymal |
| | | | | | enzymes | | |
| KRT19 | Biliary Epithelial Markers | ABCB11 | Transporters | CYP2D6 | Phase 1 metabolizing | S100A4 | EMT mesenchymal |
| | | | | | enzymes | | |
| AFP | General hepatocyte | ABCC1 | Transporters | CYP2E1 | Phase 1 metabolizing | OCLN | EMT Epithelial |
| | | | | | enzymes | | |
| DLK1 | General hepatocyte | ABCC2 | Transporters | CYP3A4 | Phase 1 metabolizing | TJP1 | EMT Epithelial |
| | | | | | enzymes | | |
| DPP4 | General hepatocyte | SLC01B3 | Transporters | CYP3A5 | Phase 1 metabolizing | CDH1 | EMT |
| | | | | | enzymes | | |
| FM02 | General hepatocyte | SLC3A1 | Transporters | CYP3A7 | Phase 1 metabolizing | FOXC2 | EMT |
| | | | | | enzymes | | |
| FM03 | General hepatocyte | TBC1D9 | Transporters | GCLC | Others | SLUG | EMT |
| IFI16 | General hepatocyte | CEBPa | Transcription factors | GCLM | Others | SNAIL | EMT |
| NAT2 | General hepatocyte | FOXA1/A2 | Transcription factors | NFE2L2 | Others | FGA | Clotting factors |
| THRSP | General hepatocyte | GATA4 | Transcription factors | CPS1 | Mitochondrial | GCKR | Carbohydrate |
| | | | | | Metabolism | | Metabolism |
| PPIA | Housekeeping | HNF1a | Transcription factors | HMGCS2 | Mitochondrial | PCK2 | Carbohydrate |
| | | | | | Metabolism | | Metabolism |
| RPLPO | Housekeeping | HNF4a | Transcription factors | CLDN1 | Junction protein | SLC37A4 | Carbohydrate |
| | | | | | | | Metabolism |
| GJA1 | Junction protein | HNF6a | Transcription factors | GJB1 | Junction protein | GGT1 | Biliary Markers |
| CYP2C19 | Phase 1 metabolizing enzymes | LIN28B | Transcription factors | PPIA | Housekeeping | SOX9 | Biliary Markers |
| CYP2C8 | Phase 1 metabolizing enzymes | Nanog | Transcription factors | RPLPO | Housekeeping | SPP1 | Biliary Markers |
| CYP2D6 | Phase 1 metabolizing enzymes | NR1I2 | Transcription factors | AAT | General hepatocyte | KRT18 | Biliary Epithelial |
| | | | | | | | Markers |
| CYP2E1 | Phase 1 metabolizing enzymes | NR1I3 | Transcription factors | AFP | General hepatocyte | KRT19 | Biliary Epithelial |
| | | | | | | | Markers |
| CYP3A4 | Phase 1 metabolizing enzymes | OCT3/4 | Transcription factors | ALB | General hepatocyte | KRT7 | Biliary Epithelial |
| | | | | | | | Markers |
| CYP3A5 | Phase 1 metabolizing enzymes | SOX17 | Transcription factors | DLK1 | General hepatocyte | KRT8 | Biliary Epithelial |
| | | | | | | | Markers |
| CYP3A7 | Phase 1 metabolizing enzymes | ASPGR1 | Receptors | DPP4 | General hepatocyte | CYP7A1 | Bile Acid Synthesis |
| GSTA1 | Phase 2 metabolizing enzymes | RXR | Receptors | FM03 | General hepatocyte | NR1H4 | Bile Acid Synthesis |
| GSTP1 | Phase 2 metabolizing enzymes | CP | Plasma Proteins | IFI16 | General hepatocyte | SLC10A1 | Bile Acid Synthesis |
| CEBPa | Transcription factors | HAMP | Plasma Proteins | NAT2 | General hepatocyte | APOF | Transporters |
| GATA4 | Transcription factors | LRP1 | Plasma Proteins | TAT | General hepatocyte | GST1M1 | Phase 2 metabolizing |
| | | | | | | | enzymes |
| HNF6a | Transcription factors | GSTA1 | Phase 2 metabolizing | TF | General hepatocyte | SULT 1A1 | Phase 2 metabolizing |
| | | | enzymes | | | | enzymes |
| LIN28B | Transcription factors | GSTA2 | Phase 2 metabolizing | THRSP | General hepatocyte | CYP1A2 | Phase 1 metabolizing |
| | | | enzymes | | | | enzymes |
| NR1I2 | Transcription factors | GSTP1 | Phase 2 metabolizing | TTR | General hepatocyte | GJA1 | Junction protein |
| | | | enzymes | | | | |
| NR1I3 | Transcription factors | UGT1A1 | Phase 2 metabolizing | VIM | General hepatocyte | ACTB | Housekeeping |
| | | | enzymes | | | | |
| ABCB1 | Transporters | CYP2A6 | Phase 1 metabolizing | APOB | Fatty Acid /cholesterol | GAPDH | Housekeeping |
| | | | enzymes | | | | |
| ABCC2 | Transporters | CYP2B6 | Phase 1 metabolizing | CCND1 | EMT nuclear B-catenin | FM02 | General hepatocyte |
| | | | enzymes | | | | |
| APOF | Transporters | CYP2C19 | Phase 1 metabolizing | MMP7 | EMT nuclear B-catenin | CDH13 | EMT Epithelial |
| | | | enzymes | | | | |
| SLC3A1 | Transporters | CYP2C8 | Phase 1 metabolizing | COL1A1 | EMT mesenchymal | GCK | Carbohydrate |
| | | | enzymes | | | | Metabolism |

TABLE 2
List of 237 liver-enriched genes (liver signatures, specificity Gini index >0.7) that are upregulated by MB-1 (Part I)

| | | | MB-1 0.5 μM | | | MB-1 5 μM | | | |
|---|---|---|---|---|---|---|---|---|---|
| Gene ID | Description | Gene Symbol | 2 hour | 24 hour | 7 day | 2 hour | 24 hour | 7 day | MAX |
| 2938 | glutathione S-transferase alpha 1 | GSTA1 | 0.24 | 4.23 | 24.58 | 1.56 | 7.48 | 31.63 | 31.63 |
| 389434 | iodotyrosine deiodinase | IYD | −1.25 | 12.21 | 12.43 | 0.75 | 21.44 | 18.22 | 21 44 |
| 7276 | transthyretin | TTR | 0.08 | 2.3 | 10.99 | −0.27 | 3.43 | 20.53 | 20.53 |
| 563 | alpha-2-glycoprotein 1, zinc-binding | AZGP1 | −0.86 | 1.5 | 13.49 | 0.75 | 3.05 | 9.89 | 19.89 |
| 27165 | glutaminase 2 | GLS2 | 1.56 | 8.8 | 6.62 | 0.53 | 18.17 | 11.68 | 18.17 |
| 118471 | proline rich acidic protein 1 | PRAP1 | −0.69 | 3.17 | 14.80 | 0.92 | 4.70 | 17.93 | 17.93 |
| 10 | N-acetyltransferase 2 | NAT2 | −084 | 3.06 | 11.30 | −1.15 | 8.45 | 16.77 | 16.77 |
| 2161 | coagulation factor XII | F12 | −0.22 | 3.94 | 11.77 | 0.05 | 8.34 | 16.35 | 16.35 |
| 1776 | deoxyribonuclease 1 like 3 | DNASE1L3 | −3.7 | 6.02 | 2.24 | 7.62 | 15.86 | 3.42 | 15.86 |
| 92292 | glycine-N-acyltransferase like 1 | GLYATL1 | −1.40 | 8.04 | 6.10 | −1.43 | 15.45 | 9.29 | 15.45 |
| 8608 | retinol dehydrogenase 16 | RDH16 | 0.13 | 6.92 | 4.23 | 1.93 | 14.76 | 6.54 | 14.76 |
| 6359 | C-C motif chemokine ligand 15 | CCL15 | 2.42 | 8.52 | 7.01 | 1.84 | 14.72 | 9.18 | 14.72 |
| 1373 | carbamoyl-phosphate synthase 1 | CPS1 | 0.27 | 4.44 | 9.95 | −0.22 | 5.96 | 14.54 | 14.54 |
| 164656 | transmembrane serine protease 6 | TMPRSS6 | −0.4 | 5.55 | 10.36 | 0.83 | 9.99 | 14.46 | 14.46 |
| 18 | 4-aminobutyrate aminotransferase | ABAT | 0.4 | 9.12 | 4.76 | −0.05 | 14.19 | 8.35 | 14.19 |
| 8856 | nuclear receptor subfamily 1 group I | NR1I2 | 0.07 | 7.74 | 5.22 | −0.70 | 14.11 | 9.34 | 14.11 |
| | member 2 | | | | | | | | |
| 5009 | ornithine carbamoyltransferase | OTC | 2.26 | 4.51 | 10.37 | −2.45 | 8.61 | 14.06 | 14.06 |
| 64241 | ATP binding cassette subfamily G | ABCG8 | −0.07 | 8.03 | 4.34 | 1.00 | 13.95 | 5.74 | 13.95 |
| | member 8 | | | | | | | | |
| 3242 | 4-hydroxyphenylpyruvate dioxygenase | HPC | 0.78 | 5.9 | 10.14 | 1.87 | 11.69 | 13.93 | 13.93 |
| 346606 | monoacylglycerol O-acyltransferase 3 | MOGAT3 | 0.51 | 6.41 | 10.45 | 0.48 | 11.58 | 13.93 | 13.93 |
| 2203 | fructose-bisphosphatase 1 | FBP1 | −0.25 | 3.09 | 8.27 | −0.07 | 9.95 | 13.54 | 13.54 |
| 5207 | 6-phosphofructo-2-kinase/fructose- | PFKFB1 | 058 | 4.9 | 8.94 | −1.19 | 7.32 | 13.52 | 13.52 |
| | 2,6-biphosphatase 1 | | | | | | | | |
| 54898 | ELOVL fatty acid elongase 2 | ELOVL2 | 0.08 | 7.59 | 3.21 | 1.20 | 13.47 | 8.5 | 13.47 |
| 259 | alpha-1-microglobulin/bikunin | AMBP | 0.28 | 0.64 | 5.10 | −0.24 | 2.97 | 13 24 | 13.24 |
| | precursor | | | | | | | | |
| 1962 | enoyl-CoA hydratase and 3- | EHHADH | 1.00 | 5.80 | 6.83 | 0.24 | 10.39 | 13.14 | 13.14 |
| | hydroxyacyl CoA dehydrogenase | | | | | | | | |
| 335 | apolipoprotein A1 | APOA1 | −0.42 | 3.85 | 9.25 | 1.27 | 6.04 | 12.94 | 12.94 |
| 1557 | cytochrome P450 family 2 subfamily C | CYP2C19 | −0.58 | 1.79 | 4.27 | 1.02 | 2.88 | 1292 | 12.92 |
| | member 19 | | | | | | | | |
| 213 | albumin | ALB | −0.21 | 3.5 | 8.33 | −0 96 | 6.19 | 12.79 | 12.79 |
| 23475 | quinolinate phosphoribosyltransferase | QPRT | −0.41 | 7.96 | 4.77 | 0.01 | 12.78 | 7 28 | 12.78 |
| 6718 | aldo-keto reductase family 1 member | AKR1D1 | 0.83 | 6.77 | 5.29 | 1.03 | 12.61 | 7.82 | 12.61 |
| | D1 | | | | | | | | |
| 6718 | aldo-keto reductase family 1 member | AKR1D1 | 0.83 | 6.77 | 5.29 | 1.03 | 12.61 | 782 | 12.61 |
| | D1 | | | | | | | | |
| 83758 | retinol binding protein 5 | RBP5 | 0.78 | 5.76 | 8.97 | 0.19 | 7.48 | 12.56 | 12.56 |
| 10157 | aminoadipate-semialdehyde synthase | AASS | −1.29 | 2.2 | 829 | 1.02 | −0.97 | 12 39 | 12.39 |
| 1244 | ATP binding cassette subfamily C | ABCC2 | 0.2 | 4.37 | 4.32 | −1.98 | 12.21 | 5.35 | 12.21 |
| | member 2 | | | | | | | | |
| 54490 | UDP glucuronosyltransferase family 2 | UGT2B28 | 0.96 | 4.63 | 4.9 | 1.59 | 12.19 | 8.06 | 12.19 |
| | member B28 | | | | | | | | |
| 26998 | fetuin B | FETUB | −1.85 | 5.16 | 8.24 | −0.24 | 9.52 | 11.90 | 11.90 |
| 92840 | receptor accessory protein 6 | REEP6 | 0.27 | 4.42 | 6.66 | 1.39 | 10.43 | 11.85 | 11.85 |
| 345 | apolipoprotein C3 | APOC3 | −1.41 | 3.88 | 9.75 | −0.26 | 4.65 | 11.61 | 11.61 |
| 5267 | serpin family A member 4 | SERPINA4 | −0.79 | 3.9 | 3.30 | 1.27 | 10.41 | 11.57 | 11.57 |
| 2159 | coagulation factor X | F10 | 0.29 | 3.23 | 7.45 | 1.59 | 3.15 | 11.56 | 11.56 |
| 54576 | UDP glucuronosyltransferase family 1, | UGT1A8 | 1.14 | 0.42 | 4.76 | −0.39 | 1.86 | 11.35 | 11.35 |
| | polypeptide A8 [ | | | | | | | | |
| 875 | cystathionine-beta-synthase | CBS | 0.28 | 2.10 | 5.82 | 1.09 | 3.2 | 11.35 | 11.35 |
| 9970 | nuclear receptor subfamily 1 group I | NR1I3 | −1.49 | 6.49 | 6.95 | 1.45 | 11.34 | 10.54 | 11.34 |
| | member 3 | | | | | | | | |
| 2819 | glycerol-3-phosphate dehydrogenase | GPD1 | −1.12 | 1.91 | 8.31 | −0.40 | 5.76 | 11.29 | 11.29 |
| | 1 | | | | | | | | |
| 6822 | sulfotransferase family 2A member 1 | SULT2A1 | −1.32 | 6.13 | 3.35 | 0 82 | 11.04 | 5.41 | 11.04 |
| 2940 | glutathione S-transferase alpha 3 | GSTA3 | 1.71 | 3.68 | 6.36 | 1.49 | 5.57 | 10.99 | 10.99 |
| 8424 | gamma-butyrobetaine hydroxylase 1 | BBOX1 | −0.69 | 5.44 | 3.71 | 0.19 | 10.78 | 1.92 | 10.78 |
| 58510 | proline dehydrogenase 2 | PRODH2 | −1.37 | 4.60 | 5.40 | −0.75 | 10.68 | 7.92 | 10.68 |
| 220001 | von Willebrand factor C and EGF | VWCE | 0.90 | 5.79 | 3.99 | 2.04 | 10.67 | 6.75 | 10.67 |
| | domains | | | | | | | | |
| 570 | bile acid-CoA:amino acid N- | BAAT | 0.03 | 4.42 | 4.35 | 0.11 | 10.59 | 4.53 | 10.59 |
| | acyltransferase | | | | | | | | |
| 570 | bile acid-CoA:amino acid N- | BAAT | 0.03 | 4.42 | 4.35 | 0.11 | 10.59 | 453 | 10.59 |
| | acyltransferase | | | | | | | | |
| 27329 | angiopoietin like 3 | ANGPTL3 | −1.25 | 6.17 | 7.93 | 1.56 | 10.33 | 10.39 | 10.39 |
| 1733 | iodothyronine deiodinase 1 | DIO1 | −0.88 | 6.04 | 8.02 | 0.51 | 8.74 | 10.37 | 10.37 |
| 79799 | UDP glucuronosyltransferase family 2 | UGT2A3 | 0.52 | 3.51 | 7.23 | 0.7 | 7.07 | 10.32 | 10.32 |
| | member A3 | | | | | | | | |
| 84647 | phospholipase A2 group XIIB | PLA2G12B | −0.2 | 6.2 | 5.93 | 0.84 | 10.29 | 7.03 | 10.29 |
| 196410 | methyltransferase like 7B | METTL7B | 1.01 | 5.30 | 4.63 | 1.88 | 10.10 | 6.02 | 10.10 |
| 462 | serpin family C member 1 | SERPINC1 | −0.72 | 1.97 | 7.63 | 0 86 | 2.33 | 10.03 | 10.03 |
| 9388 | lipase G, endothelial type | LIPG | −0.30 | 5.09 | 5.95 | −1.81 | 10.00 | 7.9 | 10.00 |
| 6554 | solute carrier family 10 member 1 | SLC10A1 | −1.72 | 4.34 | 8.83 | 0.52 | 3.78 | 9.94 | 9.94 |
| 6554 | solute carrier family 10 member 1 | SLC10A1 | −1.72 | 4.34 | 8.83 | 0.52 | 3 78 | 9.94 | 9.94 |
| 344 | apolipoprotein C2 | APOC2 | 1.89 | 2.47 | 8.77 | 1.04 | 3.30 | 9.93 | 9.93 |
| 1576 | cytochrome P450 family 3 subfamily A | CYP3A4 | −0.34 | 6.2 | 5.13 | 1.69 | 9.52 | 9.89 | 9.89 |
| | member 4 | | | | | | | | |
| 5264 | phytanoyl-CoA 2-hydroxylase | PHYH | 0.07 | 5.45 | 6.63 | 0.27 | 5.89 | 9.86 | 9.86 |
| 5002 | solute carrier family 22 member 18 | SLC22A18 | −0.46 | 4.45 | 8.07 | 1.62 | 5.91 | 9.78 | 9.78 |
| 6539 | solute carrier family 6 member 12 | SLC6A12 | −0.47 | 5.16 | 2.52 | 0.46 | 9.76 | 3.59 | 9.76 |
| 51268 | pipecolic acid and sarcosine oxidase | PIPOX | −0.32 | 5.58 | 7.01 | 0.49 | 8.68 | 9.75 | 9.75 |
| 80168 | monoacylglycerol O-acyltransferase 2 | MOGAT2 | −0.69 | 5.78 | 3.70 | −1.22 | 9.69 | 5.70 | 9.69 |
| 216 | aldehyde dehydrogenase 1 family member A1 | ALDH1A1 | −0.6 | 4.83 | 5.76 | 0.41 | 8.76 | 9.68 | 9.68 |
| 23498 | 3-hydroxyanthranilate 3,4-dioxygenase | HAAO | 0.78 | 4.78 | 4.84 | 2.34 | 9.44 | 9.26 | 9.44 |
| 7498 | xanthine dehydrogenase | XDH | 0.3 | 0.31 | 3.40 | 0.37 | 2.30 | 9.37 | 9.37 |
| 3990 | lipase C, hepatic | LIPC | 0.29 | 5.86 | 3.68 | 2.37 | 9.27 | 5.6 | 9.27 |
| 3172 | hepatocyte nuclear factor 4 alpha | HNF4A | 0.29 | 5.58 | 5.67 | 0.13 | 9.11 | 7.48 | 9.11 |
| 2165 | coagulation factor XIII B chain | F13B | −0.47 | 3.88 | 9.06 | −0.76 | 4.46 | 9.10 | 9.10 |
| 635 | betaine-homocysteine S-methyltransferase | BHMT | 1.99 | 5.00 | 4.67 | 2.85 | 9.06 | 9.03 | 9.06 |
| 1551 | cytochrome P450, family 3, subfamily A, polypeptide 7 | CYP3A7 | −0.70 | 5.2 | 5.83 | 0.10 | 8.99 | 8.28 | 8.99 |
| 1579 | cytochrome P450, family 4, subfamily A, polypeptide 11 | CYP4A11 | −0.61 | 2.82 | 5.32 | 0.19 | 8.96 | 3.8 | 8.96 |
| 5919 | retinoic acid receptor responder (tazarotene induced) 2 | RARRES2 | 0.23 | 2.74 | 5.37 | 1.17 | 6.31 | 8.94 | 8.94 |
| 8309 | acyl-CoA oxidase 2, branched chain | ACOX2 | 0.70 | 5.29 | 2.92 | 2.62 | 8.93 | 3.63 | 8.93 |
| 8630 | hydroxysteroid (17-beta) dehydrogenase 6 | HSD17B6 | 1.27 | 5.32 | 3.02 | 0.87 | 8.90 | 4.07 | 8.90 |
| 8630 | hydroxysteroid (17-beta) dehydrogenase 6 homolog (mouse) | HSD17B6 | 1.27 | 5.32 | 3.02 | 0.87 | 8.90 | 4.07 | 8.90 |
| 197 | alpha-2-HS-glycoprotein | AHSG | 0.08 | 3.31 | 7.12 | 0.65 | 2.98 | 8.89 | 8.89 |
| 8991 | selenium binding protein 1 | SELENBP1 | 0.43 | 4.15 | 6.03 | 0.15 | 7.85 | 8.87 | 8.87 |
| 10840 | aldehyde dehydrogenase 1 family, member L1 | ALDH1L1 | 0.01 | 3.85 | 5.17 | −1.04 | 6.17 | 8.79 | 8.79 |
| 27141 | cell death-inducing DFFA-like effector b | CIDEB | 0.43 | 3.69 | 8.05 | −0.07 | 4.98 | 8.77 | 8.77 |
| 79814 | agmatine ureohydrolase (agmatinase) | AGMAT | −0.66 | 5.03 | 4.68 | 0.22 | 8.77 | 6.46 | 8.77 |
| 6568 | solute carrier family 17 (organic anion transporter), member 1 | SLC17A1 | 0.76 | −1.55 | 1.84 | 1.46 | −1.37 | 8.76 | 8.76 |
| 6694 | secreted phosphoprotein 2,24 kDa | SPP2 | 0.57 | 0.07 | 2.89 | 0.04 | 0.86 | 8.72 | 8.72 |
| 5313 | pyruvate kinase, liver and RBC | PKLR | −0.61 | 5.00 | 3.99 | 1.44 | 8.71 | 4.6 | 8.71 |
| 229 | aldolase B, fructose-bisphosphate | ALDOB | 1.11 | 4.58 | 4.25 | 2.49 | 8.69 | 6.73 | 8.69 |
| 3931 | lecithin-cholesterol acyltransferase | LCAT | −0.05 | 1.88 | 5.09 | 2.47 | 4.90 | 8.64 | 8.64 |
| 6360 | chemokine (C-C motif) ligand 16 | CCL16 | −0.94 | 6.39 | 8.61 | −1.38 | 6.68 | 8.38 | 8.6 |
| 57733 | glucosidase, beta, acid 3 | GBA3 | 0.07 | 4.33 | 7.05 | 1.30 | 6.27 | 8.60 | 8.60 |
| 57733 | glucosidase, beta, acid 3 (cytosolic) | GBA3 | 0.07 | 4.33 | 7.05 | 1.30 | 6.27 | 8.60 | 8.60 |
| 1559 | cytochrome P450, family 2, subfamily C, polypeptide 9 | CYP2C9 | −0.97 | 2.30 | 2.13 | −0.82 | 8.54 | 6.65 | 8.54 |
| 10991 | solute carrier family 38, member 3 | SLC38A3 | 0.54 | 5.02 | 5.46 | 2.1 | 7.14 | 8.51 | 8.51 |
| 55532 | solute carrier family 30, member 10 | SLC30A10 | 0.49 | 5.01 | 3.38 | 1.22 | 8.49 | 7.97 | 8.49 |
| 54658 | UDP glucuronosyltransferase 1 family, polypeptide A1 | UGT1A1 | 0.77 | 3.61 | 0.79 | −1.06 | 8.41 | 5.00 | 8.41 |
| 5444 | paraoxonase 1 | PON1 | −2.09 | 3.44 | 4.69 | 0.10 | 8.29 | 7.53 | 8.29 |
| 2998 | glycogen synthase 2 (liver) | GYS2 | 1.14 | 4.68 | 4.38 | 2.43 | 8.24 | 2.97 | 8.24 |
| 4547 | microsomal triglyceride transfer protein | MTTP | −1.41 | 6.74 | 3.92 | −2.34 | 8.19 | 4.85 | 8.19 |
| 64816 | cytochrome P450, family 3, subfamily A, polypeptide 43 | CYP3A43 | −0.75 | 4.43 | 3.70 | 0.24 | 8.09 | 6.23 | 8.09 |
| 5950 | retinol binding protein 4, plasma | RBP4 | −1.44 | 1.93 | 5.77 | 0.6 | 3.07 | 8.04 | 8.04 |
| 55937 | apolipoprotein M | APOM | −1.22 | 1.88 | 5.46 | −0.34 | 2.25 | 7.97 | 7.97 |
| 55244 | solute carrier family 47, member 1 | SLC47A1 | −1.05 | 4.37 | 5.17 | 0.07 | 6.16 | 7.96 | 7.96 |
| 646282 | alpha-2-glycoprotein 1, zinc-binding pseudogene 1 | AZGP1P1 | −0.16 | 2.78 | 5.24 | −0.39 | 3.70 | 7.96 | 7.96 |
| 10864 | solute carrier family 22 (organic anion transporter), member 7 | SLC22A7 | 0.08 | 5.12 | 3.82 | 0.44 | 7.95 | 4.16 | 7.95 |
| 197257 | lactate dehydrogenase D | LDHD | −0.28 | 4.87 | 4.34 | 0.18 | 7.87 | 7.20 | 7.87 |
| 6999 | tryptophan 2,3-dioxygenase | TDO2 | −1.32 | 5.69 | 3.47 | 0.86 | 7.87 | 3.89 | 7.87 |
| 6296 | acyl-CoA synthetase medium-chain family member 3 | ACSM3 | −0.52 | 2.00 | 5.20 | 0.16 | 2.18 | 7.70 | 7.70 |
| 2705 | gap junction protein, beta 1, 32 kDa | GJB1 | 0.24 | 4.12 | 2.72 | 0.76 | 7.65 | 4.6 | 7.65 |
| 368 | ATP-binding cassette, sub-family C (CFTR/MRP), member 6 | ABCC6 | 0.06 | 4.68 | 3.08 | 2.24 | 7.57 | 4.54 | 7.57 |
| 54988 | acyl-CoA synthetase medium-chain family member 5 | ACSM5 | −0.81 | 3.08 | 4.89 | −0.49 | 5.92 | 7.55 | 7.55 |
| 55908 | chromosome 19 open reading frame 80 | C19Orf80 | 0.30 | 3.28 | −0.67 | 1.99 | 7.55 | 0.34 | 7.55 |
| 5624 | protein C (inactivator of coagulation factors Va and Villa) | PROC | 0.06 | 4.22 | 2.37 | 1.08 | 7.53 | 3.42 | 7.53 |
| 130 | alcohol dehydrogenase 6 (class V) | ADH6 | −0.71 | 4.26 | 2.76 | −0.59 | 7.52 | 3.94 | 7.52 |
| 57678 | glycerol-3-phosphate acyltransferase, mitochondrial | GPAM | 0.64 | 2.91 | 3.31 | 0.38 | 6.68 | 7.47 | 7.47 |
| 1577 | cytochrome P450, family 3, subfamily A, polypeptide 5 | CYP3A5 | −0.03 | 4.42 | 5.41 | 1.54 | 7.28 | 7.46 | 7.46 |
| 1562 | cytochrome P450, family 2, subfamily C, polypeptide 18 | CYP2C18 | −0.19 | 0.55 | 0.37 | 0.25 | 7.45 | 3.29 | 7.45 |
| 9027 | N-acetyltransferase 8 (GCN5-related, putative) | NAT8 | 1.32 | 2.55 | 2.12 | 0.95 | 7.44 | 6.11 | 7.44 |
| 51733 | ureidopropionase, beta | UPB1 | 0.69 | 3.62 | 0.40 | −1.39 | 7.29 | −0.13 | 7.29 |
| 15283 | klotho beta | KLB | −2.30 | 4.06 | 2.26 | −1.29 | 7.25 | 4.45 | 7.25 |
| 140828 | long intergenic non-protein coding RNA 261 | LINC00261 | −2.00 | 2.37 | 3.26 | −1.16 | 6.71 | 7.24 | 7.24 |
| 8529 | cytochrome P450, family 4, subfamily F, polypeptide 2 | CYP4F2 | 0.32 | 3.32 | 6.30 | 0.83 | 1.51 | 7.23 | 7.23 |
| 27232 | glycine N-methyltransferase | GNMT | −1.03 | 3.14 | 6.10 | 0.62 | 1.82 | 7.22 | 7.22 |
| 3273 | histidine-rich glycoprotein | HRG | 0.19 | 2.67 | 5.58 | 2.06 | 3.83 | 7.21 | 7.21 |
| 5053 | phenylalanine hydroxylase | PAH | 1.02 | 4.13 | 5.46 | 0.4 | 6.52 | 7.18 | 7.18 |
| 3795 | ketohexokinase (fructokinase) | KHK | −0.65 | 3.32 | 2.79 | 0.50 | 7.14 | 4.52 | 7.14 |
| 83597 | receptor (chemosensory) transporter protein 3 | RTP3 | −0.39 | 2.52 | 3.87 | 0.08 | 4.04 | 7.10 | 7.10 |
| 2538 | glucose-6-phosphatase, catalytic subunit | G6PC | 0.39 | 4.25 | 3.03 | 1.11 | 7.06 | 4.35 | 7.06 |
| 2153 | coagulation factor V (proaccelerin, labile factor) | F5 | −0.30 | 3.35 | 4.35 | 1.99 | 6.24 | 7.04 | 7.04 |
| 6580 | solute carrier family 22 (organic cation transporter), member 1 | SLC22A1 | −0.23 | 3.68 | 4.70 | 0.31 | 6.95 | 6.80 | 6.95 |
| 653190 | ATP-binding cassette, sub-family C, member 6 pseudogene 1 | ABCC6P1 | 0.38 | 4.95 | 3.48 | −0.67 | 6.91 | 5.50 | 6.91 |
| | (functional) | | | | | | | | |
| 1544 | cytochrome P450, family 1, subfamily A, polypeptide 2 | CYP1A2 | 0.23 | 1.93 | 1.56 | 1.46 | 2.91 | 6.91 | 6.91 |
| 3081 | homogentisate 1,2-dioxygenase | HGD | 0.52 | 4.25 | 2.82 | 0.09 | 6.91 | 4.66 | 6.9 |
| 79962 | DnaJ (Hsp40) homolog, subfamily C, member 22 | DNAJC22 | −0.77 | 3.08 | 2.39 | 0.10 | 6.87 | 5.13 | 6.87 |
| 64240 | ATP-binding cassette, sub-family G (WHrTE), member 5 | ABCG5 | −1.02 | 3.92 | 4.73 | 0.25 | 6.85 | 6.63 | 6.85 |
| 10786 | solute carrier family 17 (organic anion transporter), member 3 | SLC17A3 | −0.91 | −1.36 | 1.31 | 0.46 | −0.54 | 6.78 | 6.78 |
| 10786 | solute carrier family 17 (sodium phosphate), member 3 | SLC17A3 | −0.91 | −1.36 | 1.31 | −0.46 | −0.54 | 6.78 | 6.78 |
| 189 | alanine-glyoxylate aminotransferase | AGXT | −0.77 | 2.89 | 4.06 | 0.33 | 3.01 | 6.70 | 6.70 |
| 554235 | aspartate dehydrogenase domain containing | ASPDH | 0.19 | 3.12 | 3.25 | −1.62 | 6.63 | 6.66 | 6.66 |
| 2053 | epoxide hydrolase 2, cytoplasmic | EPHX2 | −1.17 | 3.83 | 2.56 | 0.70 | 6.66 | 5.28 | 6.66 |
| 3171 | forkhead box A3 | FOXA3 | 1.40 | 4.36 | 4.51 | 0.68 | 5.52 | 6.54 | 6.54 |
| 173 | afamin | AFM | 0.05 | 2.95 | 2.32 | −0.10 | 6.54 | 0.96 | 6.54 |
| 2706 | gap junction protein, beta 2, 26 kDa | GJB2 | 0.89 | 5.61 | 5.64 | 1.10 | 5.34 | 6.51 | 6.51 |
| 6514 | solute carrier family 2 (facilitated glucose transporter), | SLC2A2 | 1.84 | 3.51 | 3.20 | 1.09 | 6.50 | 4.48 | 6.50 |
| | member 2 | | | | | | | | |
| 134526 | acyl-CoAthioesterase 12 | ACOT12 | −0.29 | 3.09 | 1.64 | −1.06 | 6.49 | 2.75 | 6.49 |
| 10841 | formimidoyltransferase cyclodeaminase | FTCD | 0.04 | 4.29 | 4.57 | 1.39 | 5.77 | 6.37 | 6.37 |
| 10841 | formimidoyltransferase cyclodeaminase | FTCD | 0.04 | 4.29 | 4.57 | 1.39 | 5.77 | 6.37 | 6.37 |
| 5105 | phosphoenolpyruvate carboxykinase 1 | PCK1 | 0.54 | 3.35 | 6.37 | 2.87 | 2.39 | 5.53 | 6.37 |
| 124 | alcohol dehydrogenase 1A (class I), alpha | ADH1A | −0.31 | 2.95 | 2.72 | −0.33 | 6.36 | 4.73 | 6.36 |
| | polypeptide | | | | | | | | |
| 116519 | apolipoprotein A5 | APOA5 | 0.13 | 2.43 | 6.35 | 1.6 | 5.75 | 6.19 | 6.35 |
| 3827 | kininogen 1 | KNG1 | −0.38 | 2.74 | 4.5 | 0.43 | 2.96 | 6.34 | 6.34 |
| 1370 | carboxypeptidase N subunit 2 | CPN2 | −1.46 | 2.84 | 2.21 | −0.2 | 6.33 | 3.45 | 6.33 |
| 1491 | cystathionine gamma-lyase | CTH | 2.68 | 2 | 3.01 | 2.22 | 2.4 | 6.31 | 6.31 |
| 10555 | 1-acylglycerol-3-phosphate O-acyltransferase 2 | AGPAT2 | 0.4 | 3.09 | 6.19 | 0.59 | 3.92 | 5.81 | 6.19 |
| 131669 | urocanate hydratase 1 | UROC1 | 0.79 | 1.76 | 2.73 | 0.67 | 3.15 | 6.17 | 6.17 |
| 3818 | kallikrein B1 | KLKB1 | 0.42 | 3.88 | 2.85 | 0.95 | 6.15 | 3.23 | 6.15 |
| 127 | alcohol dehydrogenase 4 (class II), pi polypeptide | ADH4 | 1.67 | 1.41 | 2.07 | 1.03 | 3.56 | 6.11 | 6.11 |
| 7263 | thiosulfate sulfurtransferase | TST | 0.37 | 2.87 | 4.66 | 0.84 | 2.63 | 6.1 | 6.1 |
| 5340 | plasminogen | PLG | 0.13 | 1.94 | 3.95 | 0.53 | 2.52 | 6.07 | 6.07 |
| 341 | apolipoprotein C1 | APOC | 0.63 | 2.2 | 4.64 | 1.6 | 3.12 | 6.04 | 6.04 |
| 51181 | dicarbonyl and L-xylulose reductase | DCXR | 0.17 | 2.54 | 3.03 | −0.28 | 4.03 | 6.04 | 6.04 |
| 122664 | tubulin polymerization promoting protein family | TPPP2 | −2.09 | 1.4 | 2.21 | −1.47 | 3.4 | 6.01 | 6.01 |
| | member 2 | | | | | | | | |
| 1807 | dihydropyrimidinase | DPYS | −0.7 | 3.81 | 1.88 | −1.41 | 6 | 2.83 | 6 |
| 2644 | GTP cyclohydrolase I feedback regulator | GCHFR | 0.51 | 4.28 | 3.38 | 1.67 | 5.96 | 5.08 | 5.96 |
| 10249 | glycine-N-acyltransferase | GLYAT | 1.15 | −0.35 | 2.9 | −1.78 | 1.42 | 5.93 | 5.93 |
| 1 | alpha-1-B glycoprotein | A1BG | −0.25 | 1.28 | 5.91 | 0.44 | 1.01 | 4.63 | 5.91 |
| 123876 | acyl-CoA synthetase medium chain family | ACSM2A | −0.4 | 2.03 | 4.79 | −0.86 | 1.99 | 5.9 | 5.9 |
| | member 2A | | | | | | | | |
| 5174 | PDZ domain containing 1 | PDZK1 | −0.38 | 4.43 | 3.7 | 0.24 | 5.8 | 4.26 | 5.8 |
| 337 | apolipoprotein A4 | APOA4 | −0.34 | 2.09 | 2.83 | 2.32 | 5.77 | 1.33 | 5.77 |
| 761 | carbonic anhydrase 3 | CA3 | −0.32 | 2.46 | 1.63 | 0.55 | 4.66 | 5.74 | 5.74 |
| 763 | carbonic anhydrase 5A | CA5A | −1.99 | 3.26 | 4.39 | 0.91 | 4.15 | 5.73 | 5.73 |
| 445 | argininosuccinate synthase 1 | ASS1 | −0.36 | 0.89 | 4.23 | 0.73 | 1.11 | 5.72 | 5.72 |
| 151531 | uridine phosphorylase 2 | UPP2 | 0.91 | 3.18 | 3.86 | 1.64 | 4.23 | 5.66 | 5.66 |
| 130013 | aminocarboxymuconate semialdehyde | ACMSD | 0.65 | 0.49 | 3.62 | −0.17 | 2.69 | 5.65 | 5.65 |
| | decarboxylase | | | | | | | | |
| 5244 | ATP binding cassette subfamily B member 4 | ABCB4 | −0.28 | 3.71 | 3.98 | 0.12 | 5.65 | 5.32 | 5.65 |
| 866 | serpin family A member 6 | SERPINA6 | −1.33 | 3.36 | 2.37 | 1.46 | 5.6 | 3.28 | 5.6 |
| 7104 | transmembrane 4 L six family member 4 | TM4SF4 | −1.6 | 2.22 | 1.14 | 0.98 | 5.59 | 3.8 | 5.59 |
| 6898 | tyrosine aminotransferase | TAT | −0.3 | 3.46 | 3.44 | 0.93 | 5.59 | 5.32 | 5.59 |
| 7363 | UDP glucuronosyltransferase family 2 member B4 | UGT2B4 | 0.5 | 0.78 | 3.52 | 0.66 | 3.4 | 5.54 | 5.54 |
| 2160 | coagulation factor XI | F11 | −1.73 | 3.2 | 2.68 | −0.14 | 5.52 | 3.27 | 5.52 |
| 388503 | complement component 3 precursor pseudogene | C3P1 | −2.46 | 0.49 | 4.22 | −3.05 | 0.98 | 5.52 | 5.52 |
| 80781 | collagen type XVIII alpha 1 chain | COL18A1 | −0.76 | 4.9 | 4.42 | −0.3 | 4.47 | 5.46 | 5.46 |
| 129807 | neuraminidase 4 | NEU4 | 0.99 | 2.1 | 3.21 | 1.15 | 5.45 | 5.23 | 5.45 |
| 290 | alanyl aminopeptidase, membrane | ANPEP | −0.54 | 3.4 | 5.04 | 0.19 | 4.02 | 5.44 | 5.44 |
| 387601 | solute carrier family 22 member 25 | SLC22A25 | −1.87 | 3.01 | 2.04 | 1.4 | 5.41 | 3.11 | 5.41 |
| 170392 | oncoprotein induced transcript 3 | OIT3 | 0.39 | 3.43 | 3.86 | −0.6 | 5.03 | 5.41 | 5.41 |
| 81494 | complement factor H related 5 | CFHR5 | 0.08 | 1.83 | 1.71 | 1.16 | 5.41 | 0.38 | 5.41 |
| 3960 | galectin 4 | LGALS4 | −0.72 | 0.84 | 4.05 | −0.63 | 3.19 | 5.41 | 5.41 |
| 64757 | mitochondrial amidoxime reducing component 1 | MARC1 | −0.5 | 3.26 | 0.8 | −0.76 | 5.33 | 0.39 | 5.33 |
| 1548 | cytochrome P450 family 2 subfamily A member 6 | CYP2A6 | 0.34 | −1.42 | 1.67 | −0.75 | −0.8 | 5.3 | 5.3 |
| 1109 | aldo-keto reductase family 1 member C4 | AKR1C4 | −0.45 | 2.34 | 2.73 | −0.91 | 5.26 | 4.42 | 5.26 |
| 1109 | aldo-keto reductase family 1 member C4 | AKR1C4 | −0.45 | 2.34 | 2.73 | −0.9 | 5.26 | 4.42 | 5.26 |
| 8858 | protein Z, vitamin K dependent plasma | PROZ | −2.02 | 4.25 | 4.35 | −0.74 | 4.7 | 5.22 | 5.22 |
| | glycoprotein | | | | | | | | |
| 3053 | serpin family D member 1 | SERPIND1 | 0.03 | 3.07 | 3.39 | 1.38 | 3.25 | 5.21 | 5.21 |
| 3950 | leukocyte cell derived chemotaxin 2 | LECT2 | −1.55 | 1.86 | 1.45 | 1.54 | 5.16 | 4.59 | 5.16 |
| 116285 | acyl-CoA synthetase medium chain family | ACSM1 | 0.42 | 0.5 | 4.5 | −0.71 | 1.26 | 5.02 | 5.02 |
| | member 1 | | | | | | | | |
| 29974 | APOBEC1 complementation factor | A1CF | 0.38 | 2.49 | 2.27 | 2.94 | 4.95 | 2.26 | 4.95 |
| 23562 | claudin 14 | CLDN14 | −0.75 | 3.71 | 2.5 | −2.09 | 4.86 | 4.89 | 4.89 |
| 145264 | serpin family A member 12 | SERPINA12 | 0.66 | 0.75 | 1.78 | −1.11 | 3.73 | 4.83 | 4.83 |
| 91703 | aminoacylase 3 | ACY3 | −1.18 | 2.54 | 1.42 | −2.96 | 3.99 | 4.82 | 4.82 |
| 7274 | alpha tocopherol transfer protein | TTPA | −0.61 | 3.24 | 1.93 | 0.36 | 4.79 | 2.41 | 4.79 |
| 2642 | glucagon receptor | GCGR | −0.89 | −1.11 | 2.17 | −0.53 | 0.02 | 4.77 | 4.77 |
| 127845 | golgi transport 1A | GOLT1A | −0.91 | 1.5 | 2.24 | 1.63 | 4.75 | 4.18 | 4.75 |
| 10878 | complement factor H-related 3 | CFHR3 | 4.06 | 2.58 | 1.03 | 4.74 | 1.11 | −1.77 | 4.74 |
| 10747 | mannan binding lectin serine peptidase 2 | MASP2 | −0.46 | 2.08 | 4.73 | 0.18 | −1.95 | 2.45 | 4.73 |
| 114770 | peptidoglycan recognition protein 2 | PGLYRP2 | 0.03 | 1.19 | 1.59 | −0.25 | 2.45 | 4.71 | 4.71 |
| 9154 | solute carrier family 28 member 1 | SLC28A1 | −0.29 | 1.62 | 0.71 | 0.23 | 4.68 | 2.28 | 4.68 |
| 1581 | cytochrome P450 family 7 subfamily A member 1 | CYP7A1 | −1.35 | 2.25 | 1.41 | −0.8 | 4.68 | 1.71 | 4.68 |
| 10599 | solute carrier organic anion transporter family | SLCO1B1 | −2.24 | −0.86 | 2.97 | −0.35 | 4.67 | 3.93 | 4.67 |
| | member 1B1 ] | | | | | | | | |
| 1549 | cytochrome P450 family 2 subfamily A member 7 | CYP2A7 | 1.26 | 1.03 | 2.17 | 1.44 | 0.16 | 4.66 | 4.66 |
| 5104 | serpin family A member 5 | SERPINA5 | −0.91 | 2.57 | −0.19 | −0.77 | 4.64 | 3.84 | 4.64 |
| 7018 | transferrin | TF | 0.18 | 2.88 | 2.94 | −1.95 | 1.91 | 4.61 | 4.6 |
| 1565 | cytochrome P450 family 2 subfamily D member 6 | CYP2D6 | 0.44 | 0.67 | 0.74 | −0.96 | 4.59 | 3.06 | 4.59 |
| 10998 | solute carrier family 27 member 5 | SLC27A5 | −0.63 | 1.1 | 3.02 | 0.21 | 3.51 | 4.59 | 4.59 |
| 2328 | flavin containing monooxygenase 3 | FMO3 | 0.94 | 0.6 | 1.89 | −1.2 | 0.89 | 4.58 | 4.58 |
| 3697 | inter-alpha-trypsin inhibitor heavy chain 1 | ITIH1 | −0.35 | 3.52 | 2.09 | 0.74 | 4.56 | 2.47 | 4.56 |
| 13 | arylacetamide deacetylase | AADAC | −2.02 | 2.33 | 2.31 | −1.99 | 4.52 | 1.62 | 4.52 |
| 5092 | pterin-4 alpha-carbinolamine dehydratase 1 | PCBD1 | 0.66 | 0.52 | −1.6 | 1.15 | 4.51 | −0.28 | 4.51 |
| 1036 | cysteine dioxygenase type 1 | CDO1 | 0.35 | 4.31 | 1.15 | −0.7 | 4.5 | 0.83 | 4.5 |
| 51085 | MLX interacting protein like | MLXIPL | 0.85 | 2.29 | 3.48 | 0.37 | 4.47 | 4.34 | 4.47 |
| 432 | asialoglycoprotein 1 | ASGR1 | −0.34 | 3.45 | 1.16 | −0.04 | 4.46 | 0.48 | 4.46 |
| 350 | apolipoprotein H (beta-2-glycoprotein I) | APOH | 0.06 | 2.08 | 2.68 | 0.28 | 3.14 | 4.44 | 4.44 |
| 1757 | sarcosine dehydrogenase | SARDH | −0.76 | 2.83 | 3.22 | 0.03 | 3.48 | 4.44 | 4.44 |
| 51179 | hydroxyacid oxidase 2 (long chain) | HAO2 | 0.47 | 1.6 | 4.42 | 0.26 | 2.21 | 4.26 | 4.42 |
| 733 | complement component 8, gamma polypeptide | C8G | −0.01 | 2.14 | 4.39 | 0.24 | 2.22 | 3.23 | 4.39 |
| 4051 | cytochrome P450, family 4, subfamily F, polypeptide 3 | CYP4F3 | 0.18 | 4.38 | 3.27 | 0.43 | 2.79 | 3.58 | 4.38 |
| 257407 | chromosome 2 open reading frame 72 | C2orf72 | −2.19 | 1.88 | 1.98 | −1.32 | 4.34 | 2.91 | 4.34 |
| 3175 | one cut homeobox 1 | ONECUT1 | −1.7 | 0.35 | −0.26 | 0.33 | 4.28 | 2.74 | 4.28 |
| 3294 | hydroxysteroid (17-beta) dehydrogenase 2 | HSD17B2 | −2.09 | −0.29 | 0.08 | −0.3 | 4.24 | −1.55 | 4.24 |
| 3158 | 3-hydroxy-3-methyglutaryl-CoA synthase 2 | HMGCS2 | −0.74 | 2.79 | 3.57 | 0.03 | 3.2 | 4.22 | 4.22 |
| | (mitochondrial) | | | | | | | | |
| 1644 | dopa decarboxylase (aromatic L-amino acid decarboxylase) | DDC | −2.08 | 3.42 | 3.49 | −3.45 | 4.21 | 3.81 | 4.21 |
| 64902 | alanine-glyoxylate aminotransferase 2 | AGXT2 | 0.36 | 3.82 | 4.19 | −0.01 | 3.85 | 3.78 | 4.19 |
| 10866 | HLA complex P5 (non-protein coding) | HCP5 | 1.11 | 1.69 | 1.02 | −0.1 | 4.18 | −0.06 | 4.18 |
| 55811 | adenylate cyclase 10 (soluble) | ADCY10 | −0.27 | 2.39 | 1.17 | −0.6 | 0.24 | 4.14 | 4.14 |
| 283600 | solute carrier family 25, member 47 | SLC25A47 | −2.09 | −0.44 | 1.38 | −2.5 | 0.27 | 4.08 | 4.08 |
| 2330 | flavin containing monooxygenase 5 | FMO5 | −0.8 | 3.74 | 3.79 | −0.05 | 4.02 | 3.72 | 4.02 |

Next, the inventors asked whether MB-1 treatment of HLC enabled robust HBV infection from clinical isolates. HBV particles were purified from serum of CHB individual using Nycodenz™ gradient. This step successfully separated HBV Dane particles from excess of HBsAg empty particles (see FIG. 7), and purified HBV from CHB patients was used in all infection experiments throughout this study. HLC (in 96-well plate) were treated with MB-1 (1 μM in 1% DMSO) or DMSO alone (1%) for 4 days, then were infected with HBV at a multiplicity of infection (MOI) 10. Only MB-1-treated, but not DMSO-treated HLC support HBV infection; peak of HBsAg (˜16 ng/ml) was detected by day 11 post infection (pi) (see FIG. 1E). As comparison, infection of HLC with HepG2.2.15-derived virus at similar MOI (10) or higher (100) did not result in detectable HBsAg signal (see Table 3), confirming the longstanding observation that infection using cell culture-derived HBV could only be achieved in the presence of polyethylene glycol (PEG), a chemical known for its fusogenic properties (Pontecorvo, 1977) and at very high MOIs (Gripon et al., 2002; Schreiner & Nassal, 2017).

TABLE 3
HBV infection in HLC: Comparison between patient-derived vs
HepG2.2.15-derived HBV (96-well). HLC seeded in 96-well were
infected either with patient-derived HBV, or cell culture-derived
(HepG2.2.15) virus, at the indicated MOIs, in the presence
of MB-1 or 1% DMSO. Viral kinetics (HBsAg released into culture
medium) was measured every 2 day until day 14 pi.

| | Day | | | HBsAg |
|---|---|---|---|---|
| HBV Source | pi | Condition | MOI | (ng/ml) |
| Patient- | 1 | DMSO | 10 | 0.33 +/− 0.07 |
| derived | 3 | | | 0.10 +/− 0.01 |
| | 8 | | | 0.11 +/− 0.05 |
| | 11 | | | 0.23 +/− 0.05 |
| | 14 | | | 0.13 +/− 0.03 |
| | 1 | MB-1 | | 0.34 +/− 0.06 |
| | 3 | | | 0.93 +/− 0.25 |
| | 8 | | | 5.87 +/− 1.34 |
| | 11 | | | 14.31 +/− 2.02 |
| | 14 | | | 10.39 +/− 1.46 |
| HepG2.2.15 | 1 | DMSO | | 0.05 +/− 0.01 |
| | 3 | | | 0.06 +/− 0.01 |
| | 8 | | | 0.04 +/− 0.01 |
| | 11 | | | 0.05 +/− 0.01 |
| | 14 | | | 0.06 +/− 0.01 |
| | 1 | MB-1 | | 0.05 +/− 0.01 |
| | 3 | | | 0.06 +/− 0.01 |
| | 8 | | | 0.08 +/− 0.01 |
| | 11 | | | 0.10 +/− 0.01 |
| | 14 | | | 0.09 +/− 0.01 |
| | 1 | | 100 | 414.13 +/− 1.68 |
| | 3 | | | 8.12 +/− 2.21 |
| | 8 | | | 0.36 +/− 0.11 |
| | 11 | | | 0.73 +/− 0.20 |
| | 14 | | | 0.43 +/− 0.18 |
| Uninfected | 1 | | 0 | 0.20 +/− 0.08 |
| | 3 | | | 0.17 +/− 0.07 |
| | 8 | | | 0.24 +/− 0.08 |
| | 11 | | | 0.24 +/− 0.10 |
| | 14 | | | 0.20 +/− 0.08 |

### HLCs as a Disease-Relevant Assay for HBV Drug Discovery

To be considered as a disease-relevant assay for HBV drug discovery, HLC assay ideally has to be comparable to PHH, can be miniaturized in 384-well plate, and amenable for testing clinical HBV samples of diverse GTs.

Productive HBV infection can be assessed by various viral markers that represent key steps in HBV life cycle (see FIG. 2A). Following entry of a HBV virion into hepatocytes, the viral genome (˜3.2 kb) is translocated to the nucleus and converted into a cccDNA minichromosome (Seeger and Mason, 2000). cccDNA produces four (3.5, 2.4, 2.1, and 0.7 kb) viral mRNA transcripts that are translated into hepatitis B core antigen (HBcAg), hepatitis B e antigen (HBeAg) and polymerase protein (from 3.5-kb pregenomic RNA/pgRNA); viral envelope proteins (Large, Middle, and Small or HBsAg from 2.4 and 2.1 kb mRNAs); and X protein (from 0.7 kb mRNA). The 3.5-kb pgRNA has dual roles: It serves as mRNA for the nucleocapsid and polymerase protein, and also as template for reverse transcription of the viral genome that produces relaxed circular DNA (RC-DNA) packaged into virion (Locarnini & Zoulim, 2010). Infected cells also secrete HBsAg and HBeAg. On the other hand, pgRNA and cccDNA reside within infected cells (recent studies indicated that HBV particles containing pgRNA are also circulating in the plasma, Wang et al., 2016). The levels of cccDNA and pgRNA in the livers of CHB individuals correlate with viral activity and the phase of HBV infection; thus, combined markers can be used to assess the presence and replicative activity of HBV cccDNA (Laras et al., 2006). Specific detection of cccDNA by qPCR-based assay however is a major challenge due to its very low levels (0.1-1.5 copy/cell) and the presence of excess amount of RC-DNA in the cells (21,000 copies/cell) (Nassal, 2015, Schreiner & Nassal, 2017). A digital PCR (dPCR)-based cccDNA assay was developed to address some of the limitations of qPCR. Sample treatment with T5 exonuclease efficiently removed excess of RC-DNA (Schreiner & Nassal, 2017) (see FIGS. 8A-8C), and Southern Blot assay is used to confirm dPCR activity (see FIG. 9).

The inventors infected HLC and PHH (in 96-well plate) with identical virus inoculum (MOI 40) and followed the kinetics of HBV infection based on 5 viral readouts during a 14-day assay. The levels of all HBV markers in HLC are remarkably comparable to those observed in PHH (see FIGS. 2B-2F and Table 4). Very low level of cccDNA was detected by dPCR as early as day 2 pi and reached a steady state at day 6 in PHH (day 10 in HLC) at ˜11,000-13,000 cccDNA copies/well (see FIG. 2B). At the peak of infection (day 14), the levels of pgRNA ranged between ˜963,000 to ˜1,410,000 copies/well in HLC and PHH, respectively (see FIG. 2C). Comparable infection rate between HLC and PHH was also confirmed based on viral markers released into culture medium (HBV DNA, HBsAg, and HBeAg) (see FIGS. 2D-2F and Table 4) and Southern Blot assay; the latter revealed cccDNA bands with comparable intensity in both cell types (see FIG. 2G). As typically ˜40% cells (˜40,000 cells) were infected with MOI 40 (see FIGS. 2H and 10), this indicates that HLC and PHH produce fairly equivalent amount of cccDNA (˜0.3 copy/cell) and pgRNA (24-35 copies/cell). Of note, the median copy number of cccDNA and pgRNA in the livers of CHB patients is 1.5 copies/cell (range 0.003-40 copies/cell) and 6.5 copies/cell (range 0.01-8,730 copies/cell) depending on disease stage, respectively (Laras et al., 2006).

TABLE 4
Kinetics of HBV infection in HLC and PHH (96-well plate format): HLC and PHH seeded in 96-well were infected with identical
virus inoculum (patient-derived HBV, MOI 40). Kinetics of viral infection in both cell types was measured every 2 days until
day 14 pi using various markers (HBsAg, HBeAg, HBV DNA, pgRNA, and cccDNA).

| Cell | Day | | cccDNA | pgRNA | HBV DNA | | |
|---|---|---|---|---|---|---|---|
| type | pi | Albumin (ng/ml) | (×10³ copies/well) | (×10³ copies/well) | (×10⁶ copies/ml) | HBeAg (ng/ml) | HBsAg (ng/ml) |
| HLC | 2 | 852.46 +/− 10.29 | 0.61 +/− 0.39 | 10.18 +/− 5.15 | 9.03 +/− 1.39 | 0.28 +/− 0.06 | 2.59 +/− 0.71 |
| | 6 | 920.50 +/− 21.84 | 6.36 +/− 1.29 | 45.98 +/− 71.57 | 1.63 +/− 1.91 | 0.87 +/− 0.12 | 15.91 +/− 3.39 |
| | 10 | 945.76 +/− 27.63 | 12.13 +/− 4.38 | 816.61 +/− 181.82 | 2.36 +/− 4.36 | 3.81 +/− 0.42 | 15.10 +/− 1.72 |
| | 14 | 859.66 +/− 19.56 | 13.17 +/− 1.20 | 963.11 +/− 275.93 | 3.87 +/− 3.28 | 10.96 +/− 2.40 | 37.34 +/− 4.05 |
| PHH | 2 | 809.64 +/− 26.91 | 2.17 +/− 2.26 | 19.51 +/− 0.5 | 5.42 +/− 1.05 | 0.29 +/− 0.04 | 2.65 +/− 0.66 |
| | 6 | 945.77 +/− 29.08 | 14.64 +/− 2.14 | 333.39 +/− 7.15 | 8.34 +/− 1.44 | 0.96 +/− 0.14 | 7.61 +/− 0.76 |
| | 10 | 966.67 +/− 25.47 | 11.62 +/− 2.65 | 649.41 +/− 181.18 | 2.41 +/− 6.29 | 10.15 +/− 0.50 | 38.82 +/− 4.75 |
| | 14 | 1034.86 +/− 7.66 | 11.47 +/− 4.09 | 1409.88 +/− 275.92 | 4.54 +/− 8.53 | 17.20 +/− 0.44 | 51.79 +/− 3.65 |

The inventors further showed that HLC are able to support infection of a wide range of clinical HBV isolates. Purified HBV from 17 CHB sera (GT A-D) were used to infect HLC (in 384-well plate) at MOI 40. Ten out of 17 isolates propagated very efficiently with HBsAg and HBeAg levels widely ranged between 2-150 ng/ml and 1-22 ng/ml, respectively (see FIG. 21). Marked differences in replication capacity across HBV GTs in vitro had been observed by others (Mabit et al., 1996; Sozzi et al., 2016).

The First HTS on HLC to Identify Novel cccDNA Inhibitors

A phenotypic screening in HLC infected with patient-derived HBV would theoretically increase the likelihood to discover bona fide cccDNA inhibitors, but such HTS requires rigorous feasibility assessment before it can be launched. First, HLC assay duration (14-day) is far longer than other cell-based phenotypic screenings (1-3 days) which increases the technical complexity and potential assay variability. HLC assay performance (Z′ factor) was assessed by performing ˜7,000 HBV infections (at MOI 40 in 384-well plates). The Z′ factor is a statistical measure of assay quality that takes into account both assay robustness and signal variability (standard deviation); assay with a Z′ factor >0.5 is considered highly suitable for conducting a HTS (Zhang et al., 1999). The Z′ factors of three analytes (HBsAg 0.6; HBeAg 0.45; albumin 0.8) (see FIG. 11A) provides a high degree of confidence that HLC assay is robust for HTS. To ensure assay consistency, sera from 4 CHB individuals (with equal infectivity rates in HLC) were chosen as source of virus inocula. These sera (one GT A, two GT B, and one GT C) also provide broad coverage against HBV major genotypes. Second, the inherently low levels of cccDNA and low throughput of dPCR assay made it unsuitable as a primary HTS readout. The inventors reasoned that cccDNA-active hits can subsequently be identified through their more abundant, transcriptional products (HBsAg, HBeAg, and pgRNA). HBsAg and HBeAg are translated from two different viral mRNAs, and both antigens are secreted in high abundance (HBsAg>>HBeAg) from infected cells. A multiplex assay was developed to simultaneously measure HBsAg, HBeAg, and albumin as the primary HTS readout. Albumin inhibition served as a counter screen for toxic compounds and those that potentially act as non-specific secretion inhibitors (see FIG. 11B). The second readout employed pgRNA measurement as a proxy for cccDNA transcriptional activity (Laras et al., 2006); pgRNA also present at ˜80-100-fold higher than cccDNA in HLC (see FIG. 2C), increasing assay sensitivity. The advantage of this screening cascade is that both primary readout (multiplex assay from supernatant) and secondary readout (pgRNA from cell lysates) can be performed from the same samples in 384-well plate. HBsAg/HBeAg/pgRNA-active compounds will then be tested in dPCR assay. Third, validation in PHH will build confidence in the biological relevance of HLC hits. A PHH assay was established using fresh human hepatocytes isolated from humanized uPA/SCID mice (PXB-PHH, herein called PHH) (Ishida et al., 2015). The reproducibility of HLC and PHH assays were evaluated using a reference compound tested multiple times in both cell types; HLC invariably showed lesser assay variability than PHH (see FIG. 3A). Of note, compound potency against HBsAg and HBeAg in HLC shifted 5.5 to 7.6-fold in PHH.

A schematic of the HTS assay and the screening cascade is shown in FIG. 3B. Briefly, HLC were treated with MB-1 for 4 days then infected with patient-derived HBV at MOI 40; virus inoculum was removed 24 hr later. At day 3 pi, compound library (˜247K, at 4 μM) was added to cells; fresh media and compound was replenished every 2 day. Culture media were harvested at day 14 pi and analyzed by multiplex assay; data analysis was performed with Genedata Screener software. The inventors identified ˜3,752 primary hits, defined as compounds that inhibit HBsAg and HBeAg secretion >60% with albumin inhibition <40%, representing an overall ˜1.5% hit rate (see FIG. 3C). Following hit confirmation in 12-point dose response, >85% of the hits remained active against HBsAg and HBeAg, demonstrating the reproducibility of HLC assay.

HLC Hit Validation in PHH Identified Novel cccDNA Destabilizers

To increase the efficiency of HLC hit profiling, the screening cascade were carried out in PHH (see FIG. 3B). Testing ˜1,000 of HLC hits in PHH showed that many of them are also active in PHH, however, they displayed a ˜10-fold shift in potency (average HBsAg & HBeAg IC50s are 1.36-1.46 μM in HLC and 12.05-13.5 μM in PHH) (see FIG. 3D); similar to previous observation with the reference compound (see FIG. 3A). Next, the inventors tested whether compounds that simultaneously inhibit HBeAg and HBsAg are more likely to inhibit pgRNA; indeed, >70% of them also inhibit pgRNA (see FIG. 3E), which subsequently tested for their cccDNA activity. There are at least four approaches to target cccDNA: i) prevention of cccDNA production (by blocking viral entry, or conversion from RC DNA to cccDNA following viral entry), ii) reduction of cccDNA amplification through intracellular conversion pathway, iii) silencing of cccDNA transcriptional activity through epigenetic mechanisms, and iv) destabilization of cccDNA minichromosome leading to its degradation. The first mechanism is not applicable in this study as all compound addition were performed starting at day 3 post-infection after the cccDNA pool in the infected cells has been established. The second mechanism was observed in other HBV-related virus (duck hepatitis B virus, DHBV), but it is not clear whether this mechanism also occurs in HBV in human. The third mechanism (cccDNA silencing) will reduce all cccDNA downstream products (pgRNA, HBeAg, HBsAg, and HBV DNA) but most likely will not reduce cccDNA copy number as measured by PCR-based methods (such as dPCR) and Southern Blot assay. Using dPCR assay, we identified compounds that reduced cccDNA level (cccDNA destabilizers) in PHH with IC50<10 μM; their activity was further confirmed using Southern Blot. FIG. 3F showed two examples of such compounds (compound 7 and reference compound 1) that reduced cccDNA levels up to 34-49% when added starting from day 3 pi. In summary, these results provided a proof-of-concept that HTS in a HLC assay successfully identified bona fide cccDNA destabilizers that are active against clinical HBV isolate in PHH.

Molecular Profiling Revealed that cccDNA Destabilizers Induced Broad Modulation of Host Pathways

The major challenges of phenotypic discoveries are target identification and understanding compound's mode-of-action (MOA) that may affect their safety assessment (Moffat et al., 2017). Most often, this information is not available at post HTS when hit triaging is routinely based on chemical structure (chemotype) clustering and compound potency. Small molecules can also bind to several targets (polypharmacology), increasing off-target safety risks (Peters et al., 2012). In the absence of molecular targets, phenotypic discoveries would benefit from early compound profiling at transcriptomic or phenotypic levels as part of hit prioritization to identify potential safety liabilities of hit series and to develop de-risking strategies if needed (Moffat et al., 2017).

The inventors applied a transcriptomic profiling assay to evaluate how different classes of cccDNA destabilizers modulate cellular pathways in PHH. Molecular phenotyping is a gene expression assay based on a panel of 917 pathway reporter genes that represent 154 human signalling and metabolic networks (Zhang et al., 2017). These pathway reporter genes are involved in 53% of the annotated gene-gene interactions, either acting as upstream transcriptional regulators or downstream regulatory targets in these interactions. Modulation of reporter genes expression following compound treatment allowed a multiplex view of pathways involved in various biological processes of interest, including those that lead to adverse side effects (Zhang et al., 2017). The inventors tested compound 7 and reference compound 1 (together with their respective less active isomers) and two marketed HBV drugs, entecavir (ETV, a nucleoside analog with good safety profiles) and interferon-α (an immunomodulator associated with various side effects) as controls in this assay. PHH were infected with HBV (or DMSO), and 3 days later, incubated with each drug at its 1×IC90 value for 6 hours. Total cellular RNA was extracted, and the primary responses of reporter genes against each drug were measured using AmpliSeq-RNA method. As expected, ETV induced minor changes, in line with its MOA as a direct-acting-antiviral. In contrast, Roferon strongly induced interferon-α and -λ signalling pathways and downstream pathways of IFN signalling (see FIG. 12). FIG. 4A showed principal component analysis (PCA) of compound 7 and reference compound 1. Both compounds showed two completely different PCA profiles; reference compound 1 induced a much more significant and broader response than compound 7. For each series, PCA differences were observed between active compound and its less active isomer while the presence of HBV only had minor effect. The heat map of host pathways affected by these compounds is shown in FIG. 4B. The reference compound 1 showed pleiotropic effect, it modulates various host signalling and metabolic pathways in both directions (up- and down-regulation), suggesting that this compound may potentially cause off-target effects. In contrast, compound 7 elicited more selective responses; it notably modulated two pathways, the upregulation of biological oxidation and xenobiotic metabolism, and the downregulation of caspase regulation and apoptosis.

Potency of cccDNA Destabilizers Across Different HBV Genotypes

Most of HBV in vitro studies, including evaluation of compound antiviral activity, are performed in hepatoma cell lines (HepaRG or HepG2-NTCP) infected with cell culture-derived HBV (e.g. HepG2.2.15-derived virus, GT D). As evaluation of cccDNA destabilizers was performed with patient-derived HBV GT D in PHH (see FIGS. 3F and 4A-4B), the inventors asked whether compound potency would be similar when tested against patient-derived HBV isolates from other GTs, or cell culture-derived virus (HepG2.2.15). To address the first question, PHH (in 384-well plate) were infected with four clinical HBV isolates (GT A-D, at MOI 40). At day 3 pi, cells were treated with compound 7, or DMSO, every other day until harvested and analysed at day 10 pi. Several observations are noteworthy. First, clinical HBV isolates across GTs varied in their replication capacity in PHH; as noted earlier in HLC (see FIG. 21) and also by others (Mabit et al., 1996; Sozzi et al, 2016). The GT A isolate displayed very robust replication with HBsAg & HBeAg levels reached −370 ng/ml & −58 ng/ml, respectively, followed by GT C, D, and B isolates (see FIG. 5A). For each isolate, the amounts of secreted viral antigens are in close agreement with their cccDNA levels; thus, GT A isolate had the highest amount of cccDNA (˜11,000 copies/well), followed by GT C, B, and D (see FIG. 5B). The stark differences in the amount of secreted HBsAg and cccDNA across HBV GTs could not be attributed to differences in their infectivity rates; all four isolates showed fairly comparable intracellular HBsAg and HBcAg staining in PHH (see FIG. 13). Indeed, discrepancy between HBV replication activity and its protein expression/secretion had been observed previously, particularly for HBV GT B, C, and D (Sozzi et al., 2016). Nevertheless, all four HBV isolates were equally inhibited by compound 7. Interestingly, compound 7 displayed a hierarchy of potency against various HBV markers; it was highly potent against HBV DNA (IC50s 0.020-0.025 μM), followed by HBsAg & HBeAg (IC50s 0.24-0.45 μM), pgRNA (IC50 1.48 μM), and lastly, cccDNA (IC50s 6.2-7.15 μM) (see FIGS. 5C-5D and Table 5). Thus, direct measurement of cccDNA is important for accurate assessment of cccDNA destabilizer's potency. Next, the inventors evaluated whether antiviral activity of compound 7 could be affected by the source of virus inoculum. Most HBV in vitro studies are performed in hepatoma cell lines (e.g. HepaRG or HepG2 cell lines) utilizing HepG2-derived HBV as the virus inoculum. PHH and HepaRG (Gripon et al., 2002) were infected with patient-, or HepG2.2.15-, derived HBV (note that both viruses are GT D) and treated with compound 7 starting at day 3 pi. Compound 7 was equally active in PHH and HepaRG against patient-derived HBV at MOIs tested (40 and 125), but was far less potent against HepG2.2.15-derived virus in both cell types (Table 6).

TABLE 5
Potency of cccDNA destabilizer (compound 7) against patient-derived HBV (GT A-D) in PHH. Cells were infected with each
virus (MOI 40, in triplicate, 384-well plate), and at 3 day pi, compound 7 was added at 3-fold dilutions starting from 156 μM.
Assay readouts were performed at day 10 pi to measure compound activity on HBsAg, HBeAg, HBV DNA, pgRNA and
cccDNA. Albumin is a surrogate for in vitro cellular tox.

| | Antiviral activity of compound 7 against patient−derived HBV (GT A−D) in PHH | | | | | | | | | | | | | | | In vitro tox | | |
|---|---|---|---|---|---|---|---|---|---|---|---|---|---|---|---|---|---|---|
| HBV | HBsAg IC50 (μM) | | | HBeAg IC50 (μM) | | | HBV DNA IC50 (μM) | | | pgRNA IC50 (μM) | | | cccDNA IC50 (μM) | | | albumin IC50 (μM) | | |
| GT | N | Median | SD | N | Median | SD | N | Median | SD | N | Median | SD | N | Median | SD | N | Median | SD |
| A | 4 | 0.33 | 0.09 | 4 | 0.4 | 0.09 | 3 | 0.025 | 0.007 | 3 | 3.58 | 1.3 | 3 | 7.15 | 0.1 | 4 | 20.26 | 1.81 |
| B | 4 | 0.34 | 0.03 | 4 | 0.31 | 0.03 | 3 | 0.023 | 0.002 | 3 | 3.13 | 0.57 | 3 | 6.56 | 0.85 | 4 | 19.24 | 1.67 |
| C | 4 | 0.3 | 0.08 | 4 | 0.35 | 0.04 | 3 | 0.02 | 0.003 | 3 | 1.64 | 0.14 | 3 | 6.2 | 1.71 | 4 | 23.16 | 1.84 |
| D | 4 | 0.3 | 0.06 | 4 | 0.31 | 0.02 | 3 | 0.023 | 0.004 | 3 | 1.35 | 0.52 | 3 | 6.69 | 0.77 | 4 | 23.72 | 1.32 |

Altogether, these results indicate that compound potency against HBV could be influenced by source of virus inoculum. Further tests with a large number of HBV isolates from diverse genotypes can be conducted to confirm this finding.

TABLE 6
Effect of source of virus inoculum (patient- vs HepG2.2.15-derived HBV) and cell type (PHH vs HepaRG) on compound 7

| | | | | Activity of compound 7 against patient- and HepG2.2.15-derived HBV in | | | | | | | | | | | | In vitro tox | | |
|---|---|---|---|---|---|---|---|---|---|---|---|---|---|---|---|---|---|---|
| | | | | HepaRG and PHH | | | | | | | | | | | | albumin IC50 | | |
| Cell | Virus | HBV | | HBsAg IC50 (μM) | | | HBeAg IC50 (μM) | | | pgRNA IC50 (μM) | | | cccDNA IC50 (μM) | | | (μM) | | |
| type | Source | GT | MOI | N | Median | SD | N | Median | SD | N | Median | SD | N | Median | SD | N | Median | SD |
| HepaRG | Patient | D | 40 | 3 | 0.67 | 0.01 | 3 | 0.41 | 0.1 | 3 | 1.93 | 0.02 | 3 | below LLOQ | | 3 | 23.35 | 0.63 |
| | | | 125 | 3 | 0.88 | 0.16 | 3 | 0.52 | 0.15 | 3 | 4.62 | 0.7 | 3 | 7.05 | 0.2 | 3 | 25.71 | 1.73 |
| | HepG2.2.15 | D | 40 | 3 | 9.01 | 0.24 | 3 | 6.11 | 0.76 | 3 | 16.81 | 0.71 | 3 | 25.87 | 0.72 | 3 | 24.93 | 2.15 |
| | | | 125 | 3 | 10.34 | 0.85 | 3 | 6.55 | 0.86 | 3 | 26.63 | 0.56 | 3 | 27.21 | 0.42 | 3 | 26.56 | 1.25 |
| PHH | Patient | D | 40 | 3 | 0.44 | 0.02 | 3 | 0.32 | 0.13 | 3 | 1.48 | 0.08 | 3 | 6.41 | 0.25 | 3 | 23.71 | 1.43 |
| | | | 125 | 3 | 0.8 | 0.54 | 3 | 0.57 | 0.18 | 3 | 2.87 | 1.92 | 3 | 6.78 | 0.66 | 3 | 20.49 | 0.35 |
| | HepG2.2.15 | D | 40 | 3 | 9.98 | 0.33 | 3 | 15.35 | 0.36 | 3 | 12.8 | 0.53 | 3 | 24.96 | 0.45 | 3 | 21.1 | 1.17 |
| | | | 125 | 3 | 8.98 | 0.46 | 3 | 15.86 | 0.43 | 3 | 11.28 | 0.8 | 3 | 25.16 | 0.73 | 3 | 20.17 | 1.07 |

### DISCUSSION

Despite being the 7^{th} cause of deaths in the world (Stanaway et al., 2016), WHO recognized that viral hepatitis is “largely ignored as a health and development priority until recently”. Indeed, <5% of chronic viral hepatitis worldwide is diagnosed while only ˜1% of viral hepatitis individuals received treatment (WHO, 2016). Even in the US (HBV prevalence ˜1.29 million cases), <35% of HBV infections are diagnosed and only 45% of eligible CHB patients received treatment (Buckley & Strom, 2017). Without expanded intervention, the number of people living with CHB infection is estimated to remain at the current high levels for the next 40-50 years, with a cumulative 20 millions deaths occurring between 2015 and 2030 (WHO 2016). Consequently, global strategies are required to cure HBV (Revill et al., 2016; WHO, 2016). As HBV DNA integration events into the host chromosome may occur during the early phase of infection (Mason et al., 2016), a true cure, defined as HBV eradication including intrahepatic cccDNA and integrated HBV DNA, may not be feasible (Lok et al., 2017). Instead, a functional cure which allows cessation of treatment without risk of virological relapse and of liver disease progression is deemed an attainable goal (Lok et al., 2017). A functional cure is defined as sustained, undetectable HBsAg and HBV DNA in serum after completion of a finite course of treatment, leading to resolution of residual liver injury, and a decrease risk of HCC over time. Several levels of functional cure are envisioned, including complete silencing of cccDNA transcription, elimination of cccDNA, and complete resolution of liver damage (Lok et al., 2017).

Targeting cccDNA will most likely require perturbations of the cccDNA minichromosome network. HBV hijacks host factors to establish cccDNA and to regulate its transcriptional activity. For instance, host DNA damage response system is involved in conversion of HBV RC-DNA (from incoming virions) to cccDNA in the newly infected cells (Nassal, 2015; Schreiner & Nassal, 2017). Once cccDNA is formed, it recruits histone and non-histone proteins as well as viral proteins to establish its functional unit, the minichromosome (Guo & Guo, 2015; Levrero, 2009; Nassal, 2015; Schreiner & Nassal, 2017). cccDNA minichromosome can exist in two different topology, most likely with different sets of interacting partners that relate to its transcriptional activity (Newbold et al., 1995). Conceivably, chemical perturbations of cccDNA-host interactome may lead to cccDNA instability and/or silencing of its transcriptional activity; however, the crucial interacting partners required for cccDNA stability and functions are elusive and cccDNA biology is still poorly understood. In this regard, phenotypic screening poses a powerful approach to discover novel cccDNA inhibitors in a target-agnostic manner. However, cccDNA drug discovery efforts have been hampered by the lack of robust infection systems. Notwithstanding its role as the gold standard for HBV assay, PHH is not routinely used due to its rapid dedifferentiation in culture (Frazcek et al., 2013) and large donor-to-donor variability in their susceptibility to HBV (Mabit et al., 1996). For almost three decades, HBV experimental systems had mostly been contingent on non-infection systems, such as HepG2 cell lines engineered to express HBV from a transgene (Sureau et al., 1986; Sells et al., 1987; Ladner et al., 1997; Guo et al., 2007). The discovery of HepaRG, a hepatoma cell line that supports natural HBV infection (Gripon et al., 2002), and NTCP, the HBV receptor (Yan et al, 2012) represent new tools, i.e. infection systems for HBV, that allowed studies of viral entry and cccDNA biology following natural infection. The rapid advancement of iPS technologies (Shi et al., 2017), including HLC, has enabled development of novel disease models that are expected to be more physiologically-relevant than tumor cell lines, and consequently, better recapitulate human disease biology.

The use of physiological systems in drug discovery is considered as one of the first steps to increase the translatability of preclinical findings into the clinic (Eglen & Reisine, 2011; Vincent et al., 2015; Horvath et al., 2016; Ursu et al., 2017). Indeed, the high attrition rates of new drug candidates across therapeutic areas had raised the concerns on the effectiveness of preclinical models used in drug discovery (Vincent et al., 2015; Horvath et al., 2016). For instance, during 2008-2015, the failure rates of drug candidates in phase II and III trials due to the lack of efficacy were consistently between 50-60% (Arrowsmith & Miller, 2013; Harrison, 2016). Two major drug discovery strategies, phenotypic and target-based screenings, are routinely performed in immortalized/tumor cell lines, often engineered to overexpress a molecular target of interest. A large number of tumor cell lines display substantial genetic abnormalities and altered host pathways, to the extent that there is poor correlation between cell lines and patient-derived cells (Uhlen et al., 2015; Vincent et al., 2015). Overexpression of a molecular target, aimed to provide an assay with acceptable signal-to-noise ratio, also led to an artificially high level of protein that affect pathway activation and signalling not occurred in physiological condition, resulting in discrepancy between in vitro and in vivo activity (Eglen et al., 2008). In contrast, endogenous targets in primary cells are tacitly assumed to be expressed, at the levels and within the cellular environment, that more resemble those found in human. Consequently, biological activity of compound in primary cells is expected to be more predictive for its activity in vivo (Eglen & Reisine, 2011; Vincent et al., 2015; Horvath et al., 2016; Ursu et al., 2017).

HLC could potentially represent the next generation of HBV in vitro infection system. However, existing HLC are still immature (Baxter et al., 2015; Godoy et al., 2015) and showed poor susceptibility to HBV (Shlomai et al., 2014; Kaneko et al., 2016; Samurai et al., 2017). To fully manifest the promise of HLC for HBV drug discovery, HLC maturation needs to be improved. The identification of a small molecule (MB-1) that enhances hepatic maturation of HLC represents a first step in this direction. MB-1 is not a “magic bullet”; further maturation of HLC is still needed and this most likely will require combination of several approaches including culture conditions that closely emulate liver architecture (Goldring et al., 2017). Hepatocytes in the liver are highly heterogeneous in their gene expression patterns and exhibited clear gradients based on their location within the hepatic lobule (liver zonation) (Soto-Gutierrez et al., 2017; Torre et al., 2010); ˜50% of liver genes are, in fact, zonated (Halpern et al., 2017). It may be not surprising that HLC in a monolayer culture can only emulate some, but not all of ˜500 vital functions ascribed to liver (Goldring et al., 2017).

HLC support robust HBV infection of clinical isolates from various GTs with low MOI (10-40) even in the absence of PEG (polyethylene glycol, a fusogenic agent commonly used for infection of cell culture-derived HBV) and importantly, is comparable to that observed in PHH. The use of patient-derived HBV from various GTs in drug discovery is important for several reasons. HBV GT affects viral pathogenesis, disease progression and treatment response. Mixed GT infection and inter-GT recombination, in particular among GT A and C, are increasingly recognized among CHB infections, and these may have roles in pathogenesis and treatment response as well (Lin & Kao, 2017). The inventors and others (Mabit et al., 1996; Sozzi et al., 2016) observed that HBV GTs displayed marked differences in replication activity and protein secretion; such differences may affect their susceptibility to compounds with novel MOAs. A sole reliance on one HBV GT for compound screening may potentially lead to overestimation of compound potency across HBV GTs and subtypes. In addition, laboratory strain of various pathogens are known to rapidly adapt to in vitro conditions and often lost important pathophysiological characteristics (Bukh et al., 2002; Fux et al., 2005; Horvath et al., 2016).

Performing a 14-day HTS assay on HLC is not trivial. The major advantages of tumor cell line-based HTS platforms engineered to overexpress target of interest are homogeneity and reproducibility, as almost all cells express the target of interest, providing robust and reproducible signal required for HTS. On the other hand, the reproducibility of natural HBV infection in vitro is challenging, even in PHH (Mabit et al., 1996). The present study showed that HLC assay is highly reproducible with Z′ scores 0.6 (HBsAg), 0.45 (HBeAg), and 0.8 (albumin). Of note, Z′ factor >0.5 for HTS assay is considered as a high bar for complex cellular-based assays such as those associated with iPS-derived cells (Engle & Vincent, 2014). To identify novel cccDNA inhibitors in the setting of natural infection, a screening cascade was designed based on the premise that cccDNA-active compounds could sequentially be identified through its more abundant, transcriptional products (HBsAg, HBeAg, and pgRNA). This approach successfully discovered several cccDNA-active hit series in PHH as confirmed by Southern Blot assay. Of note, others have reported that circulating pgRNA and HBV core-related antigen (HBcrAg) in the plasma of CHB individuals could be used as proxy readouts for cccDNA transcriptional activity in the liver (Wang et al., 2016; Chen et al., 2017).

Assessment of compound potency based on various HBV markers offered several important insights. First, the cccDNA destabilizer (compound 7) was equally potent against four clinical HBV isolates (GT A-D) in PHH, but displayed a hierarchy of potency against various HBV markers (HBV DNA IC50<<HBsAg & HBeAg & pgRNA IC50<cccDNA IC50). This shift in potency may reflect either the abundance/half-life of HBV marker, the dynamic range of assay, or the difficulty to inhibit the target. Indeed, cccDNA is very stable (half-life 33-57 days) in the cell (Nassal, 2015). In contrast, HBV DNA-containing virions in the blood have a short half-life (˜4.4 hours) (Murray et al., 2006) which may partly explain the higher potency of compound 7 against HBV DNA than other HBV markers. Thus, measurement of the cccDNA IC50 is critical for accurate assessment of compound potency.

Intriguingly, compound 7 was far less potent against HepG2.2.15-derived virus. While the molecular target of compound 7 is unknown, phenotypic screens often identified hits that target host factors; one may hypothesize that compound 7 may target the host factor(s) required for the maintenance and transcriptional activity of cccDNA. Indeed, upon viral entry, HBV hijacks various host factors to establish cccDNA minichromosome and to regulate its transcriptional activity (Nassal, 2015). It is plausible that the reduced potency of compound 7 against HepG2.2.15-derived HBV may reflect the differences in host factors required for cccDNA maintenance and functions of both types of viruses. HepG2.2.15-derived HBV is generated in a recombinant HepG2 cell line that is perpetually passaged under antibiotic selection. Of note, HepG2 is a human hepatoma cell line reported to have poor mimicry to primary hepatocytes (Uhlen et al., 2015 and this study, FIG. 1C). This observation is not unique to HBV. D'Aiuto et al., 2017 reported the discrepancy in compound potency against HSV-1 in monkey epithelial (Vero) cells compared to iPS-derived neurons and concluded that a number of drugs that are active in neurons would not have been identified if screening was based on Vero cells. These results highlights the importance to test compound activity against different sources of HBV, not only against cell culture-derived HBV, but also against clinical isolates of various GTs.

The discovery of compounds able to trigger partial cccDNA degradation is exciting, but also daunting without knowing their molecular targets, or potential off-target activities. Pharmacological assessment of potential safety liabilities is routinely performed by screening compounds against panels of safety-relevant targets. Attributable to the cost and throughput, such screenings are usually performed on a small number of key compounds at an advanced stage of lead optimization. Any safety finding at this point either requires considerable modifications of an already optimized compound, or even, the reason for attrition (Peters et al., 2012). Indeed, non-clinical toxicology was the highest cause of attrition for >800 preclinical compounds from 4 major pharma, accounting for 40% of the failures (Waring et al., 2015). It is therefore desirable to assess compound off-target activity early during hit selection after an HTS and during hit-to-lead phase (Peters et al., 2012; Moffat et al., 2017). As shown in this study, a transcriptomic profiling assay could be used for such purpose, not only in hepatocytes, but also in other cell types e.g. cardiomyocytes, thus broadening its application as part of in vitro toxicity tools.

In summary, the inventors provided the proof-of-concept that the HLC platform represents a paradigm change for HBV drug discovery that could potentially lead to discoveries of novel therapies for HBV cure. In parallel, continued efforts to improve hepatic maturation of HLC is needed as it will benefit not only HBV drug discovery and disease modelling, but also in vitro toxicology. As drug discovery effort is a very long process (on average, it takes 13.5 years from target identification to regulatory approval) (Paul et al., 2010) with huge investment, implementation of disease-relevant assays and other tools for safety de-risking should be initiated early and throughout compound progression to prevent costly attrition such as undesired findings discovered late in the clinic.

### Example 2: Activity of Pyrrolo[2,3-b]Pyrazine Compounds Against Patient-Derived HBV in Primary Human Hepatocytes (PHH)

Various pyrrolo[2,3-b]pyrazine compounds of formula (I) were tested for their activity against HBV in PHH (patient-derived HBV, GT D), which is the gold standard of disease-relevant HBV models, following the procedure described in Example 1.

The structures of the tested compounds and their respective compound IDs are indicated in the following:

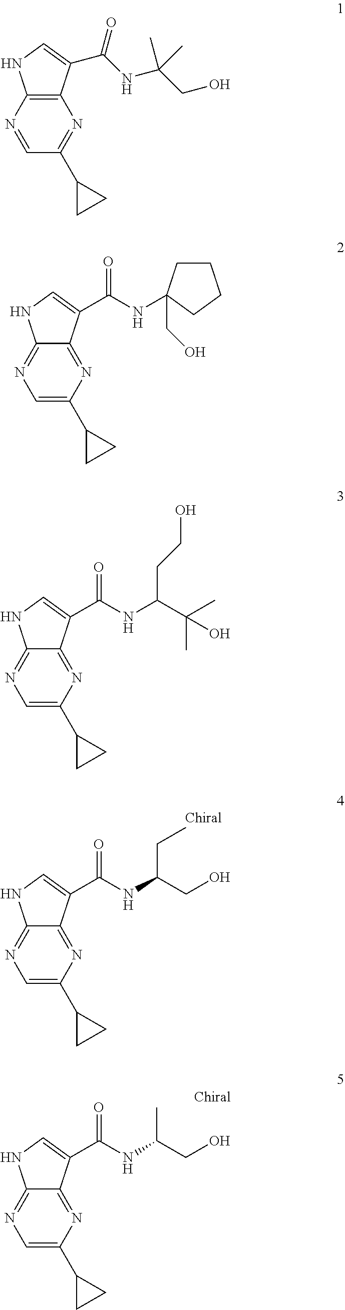 [SMILES: CC(C)(CO)NC(=O)c1c[nH]c2ncc(C3CC3)nc12 ; O=C(NC1(CO)CCCC1)c1c[nH]c2ncc(C3CC3)nc12 ; CC(C)(O)C(CCO)NC(=O)c1c[nH]c2ncc(C3CC3)nc12 ; CC[C@@H](CO)NC(=O)c1c[nH]c2ncc(C3CC3)nc12 ; C[C@H](CO)NC(=O)c1c[nH]c2ncc(C3CC3)nc12]

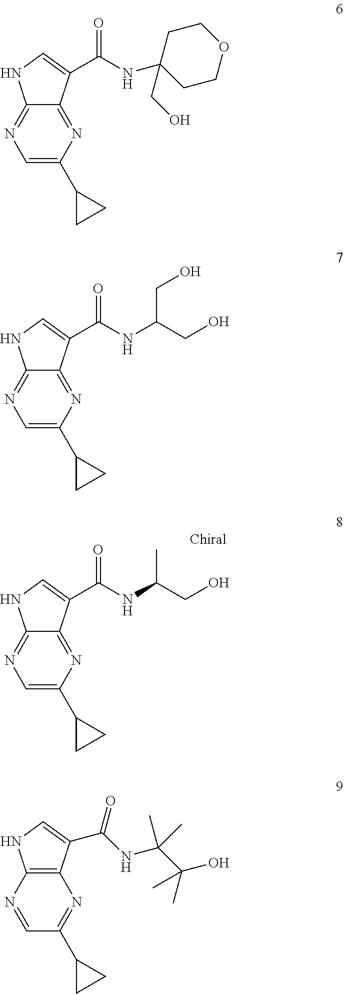 [SMILES: O=C(NC1(CO)CCOCC1)c1c[nH]c2ncc(C3CC3)nc12 ; O=C(NC(CO)CO)c1c[nH]c2ncc(C3CC3)nc12 ; C[C@@H](CO)NC(=O)c1c[nH]c2ncc(C3CC3)nc12 ; CC(C)(O)C(C)(C)NC(=O)c1c[nH]c2ncc(C3CC3)nc12]

The activities of these compounds on HBeAg, HBsAg, albumin, pgRNA and cccDNA, as determined in PHH (patient-derived HBV, GT A-D), are shown in FIGS. 15A-15D, 16D-16E and 17 as well as the following Table 7:

TABLE 7
Antiviral activity of pyrrolo[2,3-b]pyrazine compounds 1 to 9 against patient-derived HBV (GT D) in PHH.

| | Antiviral activity of pyrrolo[2,3-b]pyrazine series against patient-derived HBV (GT D) in PHH | | | | | | | | | | | | In vitro tox | | |
|---|---|---|---|---|---|---|---|---|---|---|---|---|---|---|---|
| Cmpd | HBsAg IC50 (μM) | | | HBeAg IC50 (μM) | | | pgRNA IC50 (μM) | | | cccDNA IC50 (μM) | | | albumin IC50 (μM) | | |
| ID | N | Median | SD | N | Median | SD | N | Median | SD | N | Median | SD | N | Median | SD |
| 1 | 3 | 0.17 | 0.22 | 4 | 0.17 | 0.17 | 3 | 14.21 | 1.28 | 3 | 52.67 | 3.21 | 3 | 18.68 | 1.73 |
| 2 | 5 | 0.68 | 0.37 | 6 | 0.68 | 0.21 | 3 | 0.43 | 0.09 | 4 | 30.1 | 10.97 | 4 | 24.16 | 5.36 |
| 3 | 11 | 3.62 | 0.63 | 8 | 3.58 | 0.27 | 3 | 12.81 | 1.32 | 5 | 43.73 | 4.35 | 6 | 33.42 | 6.78 |
| 4 | 7 | 0.68 | 0.54 | 4 | 0.68 | 0.09 | 3 | 6.93 | 0.82 | 3 | 63.66 | 11.72 | 4 | 47.95 | 9.64 |
| 5 | 3 | 5.61 | 0.69 | 4 | 2.95 | 0.53 | 3 | 100 | 0 | 4 | 159.6 | 9.05 | 3 | 135 | 13.45 |
| 6 | 5 | 0.75 | 0.1 | 5 | 0.49 | 0.2 | 3 | 15.67 | 1.4 | 3 | 166 | 0 | 4 | 166 | 0 |
| 7 | 11 | 0.45 | 0.59 | 13 | 0.39 | 0.24 | 3 | 1.49 | 0.09 | 10 | 6.76 | 0.73 | 13 | 20.85 | 3.32 |
| 8 | 4 | 1.22 | 0.7 | 4 | 2.29 | 0.28 | 3 | 1.96 | 0.33 | 6 | 30.75 | 1.69 | 5 | 31.9 | 8.11 |
| 9 | 8 | 0.51 | 0.28 | 6 | 0.68 | 0.21 | 3 | 1.29 | 0.24 | 4 | 49.18 | 3.74 | 6 | 21.33 | 9.21 |

N, number of repeat; SD, standard deviation.

As shown above, all of the compounds 1 to 9 according to the present invention were found to inhibit HBsAg and HBeAg with IC50 values <10 μM. Compounds 2, 4, 7, 8 and 9 showed an advantageously high inhibitory effect on pgRNA (with IC50<10 μM), and compound 7 was particularly potent in reducing cccDNA levels (with IC50<10 μM). It has thus been demonstrated that the compounds of formula (I), including in particular the above-depicted compounds 1 to 9, can be used in the treatment of HBV infection. A particularly advantageous activity against HBV has been demonstrated for compounds 2, 4, 7, 8 and 9, and especially for compound 7.

Compound 7 was further tested for its activity against patient-derived HBV GT A-D in PHH. Briefly, PHH seeded in 384-well plate were infected with patient-derived HBV (GT A-D) at MOI 40 in triplicate. At day 3 pi, compound 7 was added in 3-fold dilutions, starting at 156 μM. 1% DMSO was used as negative control. Fresh medium and compound was replenished every 2 day and cells were harvested at day 10 pi.

The results thus obtained are shown in FIGS. 16A to 16E and 17 as well as in Table 5.

Thus, compound 7 was found to exhibit potent cccDNA inhibitory activity against all 4 major HBV genotypes A to D, which further confirms that the compounds of formula (I), including in particular compound 7, allow an advantageously improved therapy of HBV infection.

### REFERENCES

Baxter, M., Withey, S., Harrison, S., Segeritz, C. P., Zhang, F., Atkinson-Dell, R., Rowe, C., Gerrard, D. T., Sison-Young, R., Jenkins, R., et al. (2015). Phenotypic and functional analyses show stem cell-derived hepatocyte-like cells better mimic fetal rather than adult hepatocytes. J. Hepatol. 62, 581-589. doi: 10.1016/j.jhep.2014.10.016.
Buckley, G. J., & Strom, B. L (eds). (2017). A National Strategy for the elimination of Hepatitis B and C: Phase Two Report. National Academies of Sciences, Engineering, and Medicine, Health and Medicine Division, Board on Population Health and Public Health Practice, Committee on a National Strategy for the Elimination of Hepatitis B and C. Washington (DC): National Academies Press (US); 2017 Mar. 28.
Bukh, J., Pietschmann, T., Lohmann, V., Krieger, N., Faulk, K., Engle, R. E., Govindarajan, S., Shapiro, M., St Claire, M., and Bartenschlager, R. (2002). Mutations that permit efficient replication of hepatitis C virus RNA in Huh-7 cells prevent productive replication in chimpanzees. Proc. Nati. Acad. Sci. USA 99, 14416-14421.
Buster, E. H. C. J., Hansen, B. E., Lau, G. K. K., Piratvisuth, T., Zeuzem, S., Steyerberg, E. W., and Janssen, H. L. A. (2009). Factors that predict response of patients with Hepatitis B e antigen-positive chronic Hepatitis B to peginterferon-alfa. Gastroenterol. 137, 2002-2009.
Cai, D., Nie, H., Yan, R., Guo, J. T., Block, T. M., and Guo, H. (2013) A southern blot assay for detection of hepatitis B virus covalently closed circular DNA from cell cultures. Methods Mol. Biol. 1030, 151-161.
Chen, C. J., Yang, H. I., Su, J., Jen, C. L., You, S. L., Lu, S. N., Huang, G. T., Iloeje, U. H., REVEAL-HBV Study Group. (2006). Risk of Hepatocellular Carcinoma Across a Biological Gradient of Serum Hepatitis B Virus DNA Level. JAMA. 295, 65-73.
Chen, E. Q., Feng, S., Wang, M. L., Liang, L. B., Zhou, L. Y., Du, L. Y., Yan, L. B., Tao, C. M., and Tang, H. (2017). Serum hepatitis B core-related antigen is a satisfactory surrogate marker of intrahepatic covalently closed circular DNA in chronic hepatitis B. Sci. Rep. 7, 173. doi: 10.1038/s41598-017-00111-0.
D'Aiuto, L., Williamson, K., Dimitrion, P., McNulty, J., Brown, C. E., Dokuburra, C. B., Nielsen, A. J., Lin, W. J., Piazza, P., Schurdak, M. E., et al. (2017). Comparison of three cell-based drug screening platforms for HSV-1 Infection. Antiviral Res. 142, 136-140. doi: 10.1016/j.antiviral.2017.03.016.
Ducluzeau, P. H., Lachaux, A., Bouvier, R., Streichenberger, N., Stepien, G., and Mousson, B. (1999). Depletion of mitochondrial DNA associated with infantile cholestasis and progressive liver fibrosis. J. Hepatol. 30, 149-155.
Easterbrook, J., Lu, C., Sakai, Y., and Li, A. P. (2001). Effects of organic solvents on the activities of cytochrome P450 isoforms, UDP-Dependent Glucuronyl transferase, and phenol sulfotransferase in human hepatocytes. Drug Met. Dis. 29, 141-144.
Eglen, R. M., Gilchrist, A., and Reisine, T. (2008). The Use of Immortalized Cell Lines in GPCR Screening: The Good, Bad and Ugly. Comb. Chem. High Throughput Screen 11, 560-565.
Eglen, R. M, and Reisine, T. (2011). Primary Cells and Stem Cells in Drug Discovery: Emerging Tools for High-Throughput Screening. Assay Drug Dev. Technol. 9(2), 108-124.
Engle, S. J, and Vincent, F. (2014). Small Molecule Screening in Human Induced Pluripotent Stem Cell-derived Terminal Cell Types. J. Biol. Chem. 289, 4562-4570.
Fraczek, J., Bolleyn, J., Vanhaecke, T., Rogiers, V., and Vinken, M. (2013). Primary hepatocyte cultures for pharmacotoxicological studies: At the busy crossroad of various anti-dedifferentiation strategies. Arch. Toxicol 87, 577-610.
Fux, C. A., Shirtliff, M., Stoodley, P., and Costerton, J. W. (2005). Can laboratory reference strains mirror ‘real-world’ pathogenesis? Trends in Microbiol. 13, 58-63.
GBD 2013 Mortality and Causes of Death Collaborators. (2015). Global, regional, and national age-sex specific all-cause and cause-specific mortality for 240 causes of death, 1990-2013: a systematic analysis for the Global Burden of Disease Study 2013. Lancet 385, 117-1171.
Godoy, P., Schmidt-Heck, W., Natarajan, K., Lucendo-Villarin, B., Szkolnicka, D., Asplund, A., Björquist, P., Widera, A., Stöber, R., Campos, G., Hammad, S., et al. (2015). Gene networks and transcription factor motifs defining the differentiation of stem cells into hepatocyte-like cells. J. Hepatol 63, 934-42. doi: 10.1016/j.jhep.2015.05.013.
Goldring, C., Antoine. D. J., Bonner, F., Crozier, J., Denning, C., Fontana, R. J., Hanley, N. A., Hay, D. C., Ingelman-Sundberg, M., Juhila, S., et al. (2017). Stem Cell-Derived Models to Improve Mechanistic Understanding and Prediction of Human Drug-Induced Liver Injury. Hepatology 65, 710-721. doi: 10.1002/hep.28886.
Gripon, P., Rumin, S., Urban, S., Le Seyec, J., Glaise, D., Cannie, I., Guyomard, C., Lucas, J., Trepo, C., and Guguen-Guillouzo, C. (2002). Infection of a human hepatoma cell line by hepatitis B virus. PNAS 99, 15655-15660.
Guenther, S., Li, B. C., Miska, S., Krueger, D. H., Meisel, H. & Will, H. (1995). A novel method for efficient amplification of whole hepatitis B virus genomes permits rapid functional analysis and reveals deletion mutants in immunosuppressed patients. J. Virol. 69, 5437-5444.
Guo, J-T., and Guo, H. (2015). Metabolism and function of hepatitis B virus cccDNA: Implications for the development of cccDNA-targeting antiviral therapeutics. Antiviral Res. 122, 91-100.
Guo, H., Jiang, D., Zhou, T., Cuconati, A., Block, T. M., and Guo, J-T. (2007). Characterization of the Intracellular Deproteinized Relaxed Circular DNA of Hepatitis B Virus: an Intermediate of Covalently Closed Circular DNA Formation. J. Virol. 81, 12472-12484.
Halpern, K. B., Shenhav, R., Matcovitch-Natan, O., Toth, B., Lemze, D., Golan, M., Massasa, E. E., Baydatch, S., Landen, S., Moor, A. E., et al. (2017). Single-cell spatial reconstruction reveals global division of labour in the mammalian liver. Nature 542, 352-356. doi: 10.1038/nature21065.
Harrison, R. K. (2016). Phase II and phase III failures: 2013-2015. Nat. Rev. Drug Discov. 15, 817-818. doi:10.1038/nrd.2016.184
Heslop, J. A., Kia, R., Pridgeon, C. S., Sison-Young, R. L., Liloglou, T., Elmasry, M., Fenwick, S. W., Mills, J. S., Kitteringham, N. R., Goldring, C. E., and Park, B. K. (2017). Donor-Dependent and Other Nondefined Factors Have Greater Influence on the Hepatic Phenotype Than the Starting Cell Type in Induced Pluripotent Stem Cell Derived Hepatocyte-Like Cells. Stem Cells Trans. Med. 6, 1321-1331.
Horvath P., Aulner N., Bickle M., Davies A. M., Nery E. D., Ebner D., Montoya M. C., Ostling P., PietiAinen V., Price L. S., et al. (2016). Screening out irrelevant cell-based models of disease. Nat. Rev. Drug Discov. 15, 751-769. doi: 10.1038/nrd.2016.175
Iloeje, U. H., Yang, H. I., Su, J., Jen, C. L., You, S. L., Chen, C. J., and the Risk Evaluation of Viral Load Elevation and Associated Liver Disease/Cancer—In HBV (the REVEAL-HBV) Study Group. (2006). Predicting cirrhosis risk based on the level of circulating hepatitis B viral load. Gastroenterology 130, 678-686.
Iloeje, U. H., Yang, H. I., Jen, C. L., Su, J., Wang, L. Y., You, S. L., and Chen, C. J. for the Risk Evaluation of Viral Load Elevation and Associated Liver Disease/Cancer-Hepatitis B Virus Study Group. (2007). Risk and Predictors of Mortality Associated With Chronic Hepatitis B Infection. Clin. Gasteroenterol. Hepatol. 5, 921-931.
Ishida, Y., Yamasaki, C., Yanagi, A., Yoshizane, Y., Fujikawa, K., Watashi, K., Abe, H., Wakita, T., Hayes, C. N., Chayama, K., and Tateno, C. 2015. Novel robust in vitro hepatitis B virus infection model using fresh human hepatocytes isolated from humanized mice. Am J Pathol. 185, 1275-85. doi: 10.1016/j.ajpath.2015.01.028.
Kajiwara, M., Aoi, T., Okita, K., Takahashi, R., Inoue, H., Takayama, N., Endo, H., Eto, K., Toguchida, J, Uemoto, S., and Yamanaka, S. (2012). Donor-dependent variations in hepatic differentiation from human-induced pluripotent stem cells. PNAS 109, 12538-12543.
Kaneko, S., Kakinuma, S., Asahina, Y., Kamiya, A., Miyoshi, M., Tsunoda, T., Nitta, S., Asano, Y., Nagata, H., Otani, S., et al. (2016). Human induced pluripotent stem cell-derived hepatic cell lines as a new model for host interaction with hepatitis B virus. Sci. Rep. 6, 29358. doi: 10.1038/srep29358.
Kumar R., Pérez-Del-Pulgar S., Testoni B., Lebossé F., and Zoulim F. (2016). Clinical relevance of the study of hepatitis B virus covalently closed circular DNA. Liver Int. 36 Suppl 1, 72-77. doi: 10.1111/liv.13001.
Kuypers, J., and Jerome, K. R. (2017). Applications of Digital PCR for Clinical Microbiology. J. Clin. Microb. pii: JCM.00211-17. doi: 10.1128/JCM.00211-17
Ladner, S. K., Otto, M. J., Barker, C. S., Zaifert, K., Wang, G. H., Guo, J. T., Seeger, C., and King, R. W. (1997). Inducible expression of human hepatitis B virus (HBV) in stably transfected hepatoblastoma cells: A novel system for screening potential inhibitors of HBV replication. Antimicrob. Agents Chemother. 41, 1715-1720.
Laras, A., Koskinas, J., Dimou, E., Kostamena, A., and Hadziyannis, S. J. (2006). Intrahepatic Levels and Replicative Activity of covalently closed circular Hepatitis B Virus DNA in chronically infected patients. Hepatology 44, 694-702.
Levrero, M., Pollicino, T., Petersen, J., Belloni, L., Raimondo, G., and Dandri, M. (2009). Control of cccDNA function in hepatitis B infection. J. Hepatol. 51, 581-592.
Lin, C. L., and Kao, J. H. (2017). Natural history of acute and chronic hepatitis B: The role of HBV genotypes and mutants. Best Pract. Res. Clin. Gastroenterol. 31, 249-255. doi: 10.1016/j.bpg.2017.04.010.
Liu, F., Wang, X. W., Chen, L., Hu, P., Ren, H., and Hu, H. D. (2016). Systematic review with meta-analysis: development of hepatocellular carcinoma in chronic hepatitis B patients with hepatitis B surface antigen seroclearance. Aliment. Pharmacol. Ther. 43, 1253-1261.
Lok, A. S., Zoulim, F., Dusheiko, G., and Ghany, M. G. (2017). Hepatitis B cure: From discovery to regulatory approval. J. Hepatol. doi: 10.1016j.jhep.2017.05.008.
Locarnini, S., and Zoulim, F. (2010). Molecular genetics of HBV infection. Antiviral Ther. 15 (Suppl 3), 3-14.
Lu, J., Einhorn, S., Venkatarangan, L., Miller, M., Mann, D. A., Watkins, P. B., and LeCluyse, E. (2015). Morphological and Functional Characterization and Assessment of iPSC-Derived Hepatocytes for In Vitro Toxicity Testing. Toxicol. Sci. 147, 39-54.
Mabit, H., Vons, C., Dubancher, S., Cape, F., Franco, D., and Petit, M. A. (1996). Primary cultured normal human hepatocytes for hepatitis B virus receptor studies. J. Hepatol. 24, 403-412.
Mason, W. S., Gill, U. S., Litwin, S., Zhou, Y., Peri, S., Pop, O., Hong, M. L., Naik, S., Quaglia, A., Bertoletti, A., and Kennedy, P. T. (2016). HBV DNA Integration and Clonal Hepatocyte Expansion in Chronic Hepatitis B Patients Considered Immune Tolerant. Gastroenterology. 151, 986-998.e4. doi: 10.1053/j.gastro.2016.07.012.
Moffat, J. G., Vincent, F., Lee, J. A., Eder, J., and Prunotto, M. (2017). Opportunities and challenges in phenotypic drug discovery: an industry perspective. Nat. Rev. Drug Discov. July 7. doi: 10.1038/nrd.2017.111.
Nassal M. (2015). HBV cccDNA: viral persistence reservoir and key obstacle for a cure of chronic hepatitis B. Gut 64, 1972-84. doi: 10.1136/gutjnl-2015-309809.
Newbold, J. E., Xin, H., Tencza, M., Sherman, G., Dean, J., Bowden, S., and Locarnini, S. (1995). The Covalently Closed Duplex Form of the Hepadnavirus Genome Exists In Situ as a Heterogeneous Population of Viral Minichromosomes. J. Virol. 69, 3350-3357.
Paul, S. M., Mytelka, D. S., Dunwiddie, C. T., Persinger, C. C., Munos, B. H., Lindborg, S. R., and Schacht, A. L. (2010). How to improve R&D productivity: the pharmaceutical industry's grand challenge. Nat. Rev. Drug Discov. 9, 203-214.
Peters, J. U., Hert, J., Bissantz, C., Hillebrecht, A., Gerebtzoff, G., Bendels, S., Tillier, F., Migeon, J., Fischer, H, Guba, W., and Kansy, M. (2012). Can we discover pharmacological promiscuity early in the drug discovery process? Drug Discov. Today. 17, 325-335. doi: 10.1016/j.drudis.2012.01.001.
Pontecorvo, G., Riddle, P. N., and Hales, A. (1977). Time and mode of fusion of human fibroblasts treated with polyethylene glycol (PEG). Nature 265, 257-258. doi:10.1038/265257a0
Rajoriya, N., Combo, C., Zoulim, F., and Janssen, H. L. A. (2017). How viral genetic variants and genotypes influence disease and treatment outcome of chronic hepatitis B. Time for an individualized approach? J. Hepatol. pii: S0168-8278(17)32154-2. doi: 10.1016/j.jhep.2017.07.011.
Revill P., Testoni B., Locarnini S., and Zoulim F. (2016). Global strategies are required to cure and eliminate HBV infection. Nat. Rev. Gastroenterol. Hepatol. 13, 239-248. doi: 10.1038/nrgastro.2016.7.
Sakurai F., Mitani S., Yamamoto T., Takayama K., Tachibana M., Watashi K., Wakita T., Iijima S., Tanaka Y., and Mizuguchi H. (2017). Human induced-pluripotent stem cell-derived hepatocyte-like cells as an in vitro model of human hepatitis B virus infection. Sci. Rep. 7, 45698. doi: 10.1038/srep45698.
Schreiner, S., and Nassal, M. (2017). A Role for the Host DNA Damage Response in Hepatitis B Virus cccDNA Formation and Beyond? Viruses 9,125. doi:10.3390/v9050125
Schweitzer, A., Horn, J., Mikolajczyk, R T., Krause, G., and Ott, J. J. (2015). Estimations of worldwide prevalence of chronic hepatitis B virus infection: a systematic review of data published between 1965 and 2013. Lancet 386, 1546-1555. doi: 10.1016/S0140-6736(16)30579-7.
Seeger, C., and Mason, W. (2000). Hepatitis B virus biology. Microbiol. Molec. Biol. Rev. 64, 51-58.
Sells, M. A., Chen, M. L., and Acs, G. (1987). Production of hepatitis B virus particles in Hep G2 cells transfected with cloned hepatitis B virus DNA. Proc. Nati. Acad. Sci. USA 84, 1005-1009.
Shi Y., Inoue H., Wu J. C., and Yamanaka S. (2017). Induced pluripotent stem cell technology: a decade of progress. Nat. Rev Drug Discov. 16, 115-130. doi: 10.1038/nrd.2016.245.
Shlomai, A., Schwartz, R. E., Ramanan, V., Bhatta, A., de Yong, Y. P., Bhatia, S. N., and Rice, C. M. (2014). Modeling host interactions with hepatitis B virus using primary and induced pluripotent stem cell-derived hepatocellular systems. PNAS 111, 12193-12198.
Soto-Gutierrez, A., Gough, A., Vernetti, L. A., Taylor, D. L., and Monga, S. P. (2017). Pre-clinical and clinical investigations of metabolic zonation in liver diseases: The potential of microphysiology systems. Exp. Biol. Med. 0, 1-12. DOI: 10.1177/1535370217707731
Sozzi, V., Walsh, R., Littlejohn, M., Colledge, D., Jackson, K., Warner, N., Yuen, L., Locarnini, S. A., and Revill, P. A. (2016). In Vitro Studies Show that Sequence Variability Contributes to Marked Variation in Hepatitis B Virus Replication, Protein Expression, and Function Observed across Genotypes. J. Virol. 90, 10054-10064.
Stanaway, J. D., Flaxman, A. D., Naghavi, M., Fitzmaurice, C., Vos, T., Abubakar, I., Abu-Raddad, L. J, Assadi, R., Bhala, N., Cowie, B., et al. (2016). The global burden of viral hepatitis from 1990 to 2013: findings from the Global Burden of Disease Study 2013. Lancet. 388, 1081-1088. doi: 10.1016/S0140-6736(16)30579-7.
Sureau, C., Romet-Lemonne, J. L., Mullins, J. I., and Essex, M. (1986). Production of hepatitis B virus by a differentiated human hepatoma cell line after transfection with cloned circular HBV DNA. Cell 47, 37-47.
Tang, H., and McLachlan, A. (2001). Transcriptional regulation of hepatitis B virus by nuclear hormone receptors is a critical determinant of viral tropism. PNAS 98, 1841-1846.
Torre, C., Perret, C., and Colnot, S. (2010). Molecular determinants of liver zonation. Prog. Mol. Biol. Transl. Sci. 97, 127-150.
Uhlen M., Fagerberg, L., Hallström B. M., Lindskog, C., Oksvold P., Mardinoglu A., Sivertsson A., Kampf C., Sjöstedt E., Asplund A., et al. (2015). Tissue-based map of the human proteome. Science 347, 1260419. doi:10.1126/science. 1260419.
Ursu, A., Schöler, H. R., and Waldmann, H. (2017). Small-molecule phenotypic screening with stem cells. Nat. Chem. Biol. 13, 560-563.
Vincent F., Loria P., Pregel, M., Stanton R., Kitching L., Nocka K., Doyonnas R., Steppan C., Gilbert A., Schroeter T., and Peakman M. C. (2015). Developing predictive assays: The phenotypic screening “rule of 3”. Sci. Transl. Med. 7, 293ps15. doi: 10.1126/scitranslmed.aab1201
Wang, J., Shen, T., Huang, X., Kumar, G. R., Chen, X., Zeng, Z., Zhang, R., Chen, R., Li, T., Zhang, T., et al. (2016). Serum hepatitis B virus RNA is encapsidated pregenome RNA that may be associated with persistence of viral infection and rebound. J. Hepatol. 65, 700-10. doi: 10.1016/j.jhep.2016.05.029.
Waring, M. J., Arrowsmith, J., Leach, A. R., Leeson P. D., Mandrell S., Owen R. M., Pairaudeau G., Pennie W. D., Pickett S. D., Wang J., Wallace O., and Weir A. (2015). An analysis of the attrition of drug candidates from four major pharmaceutical companies. Nat. Rev. Drug Discov. 14, 475-486. doi:10.1038/nrd4609.
Werle-Lapostolle, B., Bowden, S., Locarnini, S., Wursthorn, K., Petersen, J., Lau, G., Trepo, C., Marcellin, P., Goodman, Z., Delaney, W. E., et al. (2004). Persistence of cccDNA During the Natural History of Chronic Hepatitis B and Decline During Adefovir Dipivoxil Therapy. Gastroenterology 126, 1750-1758.
WHO. (2016). Global Health Sector Strategy on Viral Hepatitis, 2016-2021: Toward Ending Viral Hepatitis. WHO, Geneva, Switzerland.
Yan, H., Zhong, G., Xu, G., He, W., Jing, Z., Gao, Z., Huang, Y., Qi, Y., Peng, B., Wang, H., et al. (2012). Sodium taurocholate cotransporting polypeptide is a functional receptor for human hepatitis B and D virus. Elife 1, e00049. doi: 10.7554/eLife.00049.
Zhang, J. D., Hatje, K., Sturm, G., Broger, C., Ebeling, M., Burtin, M., Terzi, F., Pomposiello, S. I., and Badi, L. (2017). Detect tissue heterogeneity in gene expression data with BioQC. BMC Genomics 18, 277. doi: 10.1186/s12864-017-3661-2
Zhang, J. H., Chung, T. D. Y., and Oldenburg, K. R. (1999). A simple statistical parameter for use in evaluation and validation of high throughput screening assays. J. Biomol. Screen. 4, 67-73.
Zhang, X., Lu W., Zheng Y., Wang W., Bai L., Chen L., Feng Y., Zhang Z., and Yuan Z. (2016). In situ analysis of intrahepatic virological events in chronic hepatitis B virus infection. J Clin. Invest. 126, 1079-1092. doi:10.1172/JCI83339.

1. A method of treating a hepatitis B virus infection, the method comprising administering a compound of the following formula (I): 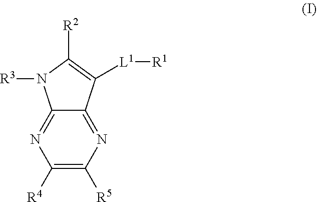
wherein:
L¹ is selected from —CO—N(R^{L1})—, —N(R^{L1})—CO—, —CO—, —N(R^{L1})—, —C(═O)O—, —O—C(═O)—, —SO—, —SO₂—, —SO₂—N(R^{L1})—, and —N(R^{L1})—SO₂—;
each R^{L1} is independently selected from hydrogen and C₁₋₅ alkyl;
R¹ is C₁₋₁₂ alkyl, C₂₋₁₂ alkenyl or C₂₋₁₂ alkynyl, wherein said alkyl, said alkenyl or said alkynyl is substituted with one or more groups R¹⁰, and further wherein said alkyl, said alkenyl or said alkynyl is optionally substituted with one or more groups R¹¹;
each R¹⁰ is independently selected from —OH, —O(C₁₋₅ alkyl), and heterocyclyl having at least one oxygen ring atom;
each R¹¹ is independently selected from —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CF₃, —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), carbocyclyl, and heterocyclyl, wherein said carbocyclyl and said heterocyclyl are each optionally substituted with one or more groups R¹²; and further wherein any two groups R¹¹ that are bound to the same carbon atom may optionally form, together with the carbon atom that they are attached to, a 5- to 8-membered carbocyclic or heterocyclic ring, wherein said 5- to 8-membered carbocyclic or heterocyclic ring is optionally substituted with one or more groups R¹²;
each R¹² is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CF₃, —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), and —(C₀₋₃ alkylene)-N(C₁₋₅ alkyl)-SO₂—(C₁₋₅ alkyl);
R² is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CF₃, —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-carbocyclyl, and —(C₀₋₃ alkylene)-heterocyclyl, wherein the carbocyclyl moiety of said —(C₀₋₃ alkylene)-carbocyclyl and the heterocyclyl moiety of said-(C₀₋₃ alkylene)-heterocyclyl are each optionally substituted with one or more groups R¹²;
R³ is selected from hydrogen, C₁₋₅ alkyl, and —CO(C₁₋₅ alkyl); and
R⁴ and R⁵ are each independently selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CF₃, —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-carbocyclyl, and —(C₀₋₃ alkylene)-heterocyclyl, wherein the carbocyclyl moiety of said —(C₀₋₃ alkylene)-carbocyclyl and the heterocyclyl moiety of said-(C₀₋₃ alkylene)-heterocyclyl are each optionally substituted with one or more groups R¹²;
or a pharmaceutically acceptable salt thereof.

2. The method of claim 1, wherein L¹ is —CO—N(R^{L1})—.

3. The method of claim 1, wherein R¹ is C₂₋₁₀ alkyl, wherein said alkyl is substituted with one or more groups R¹⁰, and further wherein said alkyl is optionally substituted with one or more groups R¹¹.

4. The method of claim 1, wherein R¹ is —C(R¹³)(R¹³)—C(R¹³)(R¹³)—R¹⁰, wherein each R¹³ is independently selected from hydrogen, methyl and ethyl, wherein each R¹³ is optionally substituted with one or more groups R¹⁰, and wherein each R¹³ is optionally further substituted with one or more groups R¹¹.

5. The method of claim 1, wherein each R¹⁰ is —OH.

6. The method of claim 1, wherein each R¹¹ is independently selected from —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CF₃, and —CN; and further wherein any two groups R¹¹ that are bound to the same carbon atom may optionally form, together with the carbon atom that they are attached to, a saturated 5- or 6-membered carbocyclic or heterocyclic ring, wherein said saturated 5- or 6-membered carbocyclic or heterocyclic ring is optionally substituted with one or more groups R¹².

7. The method of claim 1, wherein R² and R³ are each hydrogen.

8. The method of claim 1, wherein one of R⁴ and R⁵ is carbocyclyl or heterocyclyl, wherein said carbocyclyl or said heterocyclyl is optionally substituted with one or more groups R¹², and the other one of R⁴ and R⁵ is selected from hydrogen, C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CF₃, —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-carbocyclyl, and —(C₀₋₃ alkylene)-heterocyclyl, wherein the carbocyclyl moiety of said —(C₀₋₃ alkylene)-carbocyclyl and the heterocyclyl moiety of said-(C₀₋₃ alkylene)-heterocyclyl are each optionally substituted with one or more groups R¹².

9. The method of claim 1, wherein R⁵ is cyclopropyl.

10. The method of claim 1, wherein R⁴ is hydrogen.

11. The method of claim 1, wherein said compound is a compound of the following formula (II): 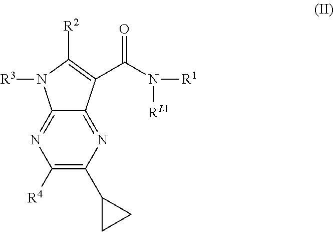
wherein the groups R¹, R^{L1}, R², R³ and R⁴ have the same meanings as in formula (I);
or a pharmaceutically acceptable salt thereof.

12. The method of claim 1, wherein said compound is a compound having any one of the following formulae, or a pharmaceutically acceptable salt thereof:

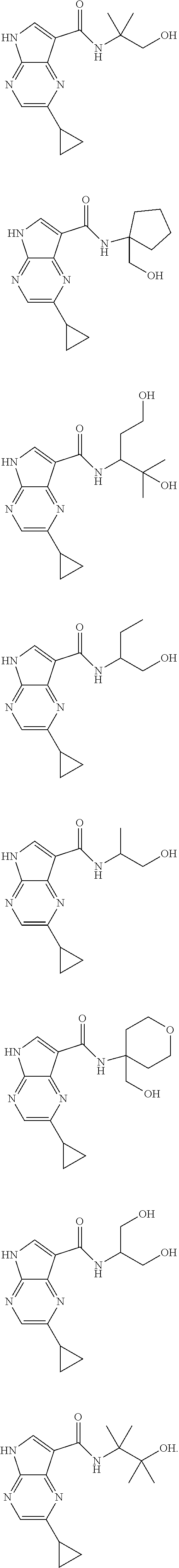

13. The method of claim 1, wherein said compound is a compound having any one of the following formulae, or a pharmaceutically acceptable salt thereof:

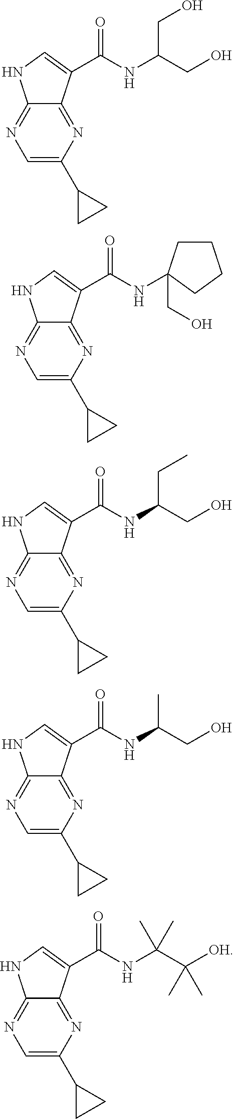

14. The method of claim 1, wherein said compound is a compound of the following formula: 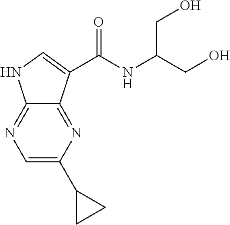 [SMILES: O=C(NC(CO)CO)c1c[nH]c2ncc(C3CC3)nc12]
or a pharmaceutically acceptable salt thereof.

15.-18. (canceled)

19. The method of claim 1, wherein said hepatitis B virus infection is a chronic hepatitis B virus infection.

20.-21. (canceled)

22. A method of treating or suppressing hepatitis B virus reactivation, the method comprising administering a compound as defined in claim 1 to a subject in need thereof.

23. The claim 1, wherein the subject to be treated is a human.

24. (canceled)

25. A method of identifying an inhibitor of hepatitis B virus (HBV) cccDNA, the method comprising:
providing stem cell-derived hepatocyte-like cells infected with HBV;
subjecting a test compound to the stem cell-derived hepatocyte-like cells infected with HBV;
determining the inhibitory effect of the test compound on HBsAg and HBeAg in the infected stem cell-derived hepatocyte-like cells;
optionally determining the inhibitory effect of the test compound on albumin in the infected stem cell-derived hepatocyte-like cells and, if the test compound has been found to inhibit albumin, excluding it from further testing;
if the test compound has been found to inhibit HBsAg and HBeAg, determining the inhibitory effect of the test compound on HBV pgRNA;
if the test compound has been found to inhibit HBV pgRNA, determining the inhibitory effect of the test compound on HBV cccDNA; and
if the test compound has been found to inhibit HBV cccDNA, selecting the test compound as an inhibitor of HBV cccDNA.

26. The method of claim 25, wherein the step of providing stem cell-derived hepatocyte-Ike cells infected with HBV comprises:
treating induced pluripotent stem cells with the compound MB-1 or MB-2 or a pharmaceutically acceptable salt thereof 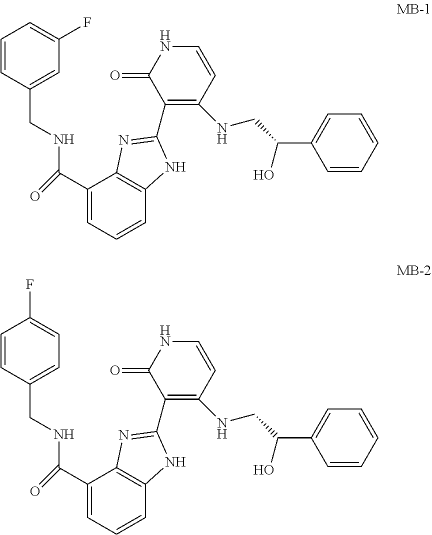
to obtain stem cell-derived hepatocyte-like cells; and
infecting the cells thus obtained with a clinical HBV isolate to obtain the stem cell-derived hepatocyte-like cells infected with HBV.

27. The method of claim 25, wherein the stem cell-derived hepatocyte-like cells are infected with clinical HBV isolates from at least the HBV genotypes A, B, C and D.
